(19) United States
(12) Patent Application Publication
(10) Pub. No.: US 2022/0233572 A1
(43) Pub. Date: Jul. 28, 2022

(54) TREATMENT OF TLR-4 MEDIATED DISEASES AND CONDITIONS WITH APTAMERS TARGETING TLR-4

(71) Applicant: APTATARGETS, S.L., Madrid (ES)

(72) Inventors: Macarena HERNÁNDEZ JIMÉNEZ, Madrid (ES); Carlos ZARAGOZA, Madrid (ES); David PIÑEIRO DEL RIO, Madrid (ES); Fernando DE CASTRO SOUBRIET, Madrid (ES); Beatriz FERNANDEZ GÓMEZ, Madrid (ES); Ignacio LIZASOAIN HERNÁNDEZ, Madrid (ES); Maria Ángeles MORO SÁNCHEZ, Madrid (ES); Lisardo BOSCÁ GOMAR, Madrid (ES); Diego PÉREZ RODRÍGUEZ, Madrid (ES); David SEGARRA DE LA PEÑA, Madrid (ES); María Eugenia ZARABOZO LEAL, Madrid (ES)

(73) Assignee: APTATARGETS, S.L., Madrid (ES)

(21) Appl. No.: 17/611,497

(22) PCT Filed: May 16, 2020
(86) PCT No.: PCT/IB2020/054655

§ 371 Date: Nov. 15, 2021

Related U.S. Application Data
(60) Provisional application No. 62/849,068, filed on May 16, 2019.
(60) Provisional application No. 62/849,072, filed on May 16, 2019.

(30) Foreign Application Priority Data
May 27, 2019 (EP) 19382424
May 27, 2019 (EP) 19382425

(51) Int. Cl. A61K 31/711; A61P 9/10

(52) U.S. Cl. CPC A61K 31/711; A61P 9/10

(57) ABSTRACT

The present disclosure related to methods to treat, prevent (e.g., suppress, inhibit or delay), or ameliorate the symptoms of a TLR-4 mediated disease or condition comprising administering an aptamer of the present disclosure to a subject in need thereof, alone or combination with other pharmacological and/or surgical interventions. In a particular aspect, the aptamers of the present disclosure are administered before, during, or after pharmacological and/or surgical interventions (e.g., thrombolysis such as thrombectomy) or any combination thereof, for the treatment of ischemic (e.g., myocardial infarction or ischemic stroke), hemorrhagic (e.g., hemorrhagic stroke or hemorrhagic transformation), or neurodegenerative (e.g., multiple sclerosis) diseases or conditions. The disclosure also provides specific doses and dosage regimes.

### FIELD

The present disclosure provides methods for the treatment of TLR-4 mediated diseases and conditions comprising administering nucleic acid aptamers specifically targeting the extracellular domain of TLR-4.

### BACKGROUND ART

Toll-like receptors (TLRs) are a family of pattern-recognition receptors initially identified for their role in the activation of innate immunity that can also control the activation of adaptive immune responses. TLR-4 was the first TLR characterized in mammals. The most important endogenous TLR-4 ligands are molecules released in response to tissue or cell damage. Thus, TLR-4 is involved in a number of highly prevalent pathologies related to tissue of cell damage, such as stroke.

The involvement of innate immunity and, in particular, of TLRs in multiple pathologies has sparked growing interest in the development of agonists and antagonists of these receptors as pharmacological targets. However, drugs able to modulate TLR-4 are scarce; furthermore, in general drugs able to modulate TLR-4 and treat or prevent TLR-4 mediated currently under development are appropriate for the treatment of a certain condition or a limited number of conditions. Thus, there is a need in the art for broad spectrum molecules with the capability of binding specifically to and inhibiting TLR-4 and that are useful as therapeutic agents against a broad range of diseases and conditions related to the overexpression or overactivation of TLR-4.

### BRIEF SUMMARY

The present disclosure provides an aptamer for use in ameliorating or improving at least a symptom or sequelae of acute cardiac infarction, wherein

(a) the aptamer has a length between 40 and 100 nucleotides and is selected from the group consisting of SEQ ID NOS: 1, 2, 3, and 4 (or any aptamer sequence of TABLE 1 or a combination thereof), wherein
(i) the aptamer specifically binds to an epitope on the extracellular domain of TLR-4; and,
(ii) binding of the aptamer to the epitope reduces and/or inhibits TLR-4 activation; or

(b) the aptamer is a functional equivalent variant of the aptamer of (a) having at least 85% sequence identity to SEQ ID NO: 1, 2, 3, or 4 (or any aptamer sequence of TABLE 1 or a combination thereof), wherein the functionally equivalent variant is derived from SEQ ID NO: 1, 2, 3, or 4 (or any aptamer sequence of TABLE 1 or a combination thereof), and maintains the capability of specifically binding to and reducing and/or inhibiting TLR-4 activation; and wherein

the aptamer is administered during, prior, or immediately after the acute cardiac infarction.

In an embodiment, the administration of the aptamer causes a reduction of infarct area, and particularly, a reduction of infarct area of that least 25% compared to control conditions.

In another embodiment, the administration of the aptamer causes a decrease in fibrosis and/or necrosis caused by the acute cardiac infarction.

In another embodiment, the administration of the aptamer results in

(i) improvement in cardiac function;

(ii) reduction of degradation of extracellular matrix;

(iii) improvement in cardiac remodeling;

(iv) preservation in ventricular anatomy;

(v) reduction of progression of the infarction; or

(vi) any combination thereof.

The present disclosure also provides the aptamer as defined above, for use in ameliorating or improving at least a symptom or sequelae of a neuromuscular or neurodegenerative disease or condition, wherein the aptamer is administered during, prior, or after the onset of the neuromuscular or neurodegenerative disease or condition.

In one embodiment, the administration of the aptamer causes

(i) reduction in demyelination;

(ii) reduction in axonal damage; or,

(iii) a combination thereof.

In another embodiment, the administration of the aptamer causes an inhibition of demyelination of at least 20-80% compared to control conditions (e.g., administration of placebo).

In another embodiment, the administration of the aptamer causes a reduction in (i.e., protection against) axonal damage of at least 10-30% compared to control conditions (e.g., administration of placebo).

In some embodiments, the neuromuscular or neurodegenerative disease or condition is selected from the group consisting of amyotrophic lateral sclerosis (ALS), Parkinson's disease, Huntington's disease, Alzheimer's disease, and vascular dementia disease.

In some embodiments, the aptamer used in the treatments is ApTOLL. In other embodiments, the aptamer is administered at a dose range between about 0.5 mg/dose and about 14 mg/dose. In some embodiments, the aptamer is administered at a dose range between about 0.007 mg/kg per dose and about 0.2 mg/kg per dose. In some embodiments, the aptamer is formulated in PBS (sodium chloride, potassium chloride, disodium hydrogen phosphate dehydrate, and potassium dihydrogen phosphate) pH 7.4, comprising magnesium chloride hexahydrate, and optionally comprising A-trehalose dihydrate. In an embodiment, the aptamer is administered intravenously by infusion.

The present disclosure also provides methods of treating TLR-4 mediated diseases and conditions (e.g., myocardial infarction, hemorrhagic stroke, hemorrhagic transformation, or multiple sclerosis) stroke in a subject in need thereof comprising administering to the subject at least one dose of a nucleic acid aptamer 40 to 80 nucleobases in length, wherein the aptamer binds to an epitope on the extracellular domain of TLR-4, and wherein binding of the aptamer to the epitope reduces and/or inhibits TLR-4 activation. In some aspects, binding of the aptamer to the epitope reduces TLR-4 activation. In some aspects, binding of the aptamer to the epitope inhibits TLR-4 activation.

In some aspects, the method further comprises administering an additional treatment or a combination thereof. In some aspects, the additional treatment is a second TLR-4 antagonist. In some aspects, the additional treatment is a surgical intervention. In some aspects, the additional treatment comprises the administration of an anti-inflammatory agent, a nucleic acid, a peptide, or a combination thereof. In some aspects, the peptide comprises an antibody or an antigen-binding fragment thereof. In some aspects, the nucleic acid comprises an antisense oligonucleotide, an antimir, a siRNA, or an shRNA.

In some aspects, the nucleic acid aptamer comprises a sequence at least 70% identical to SEQ ID NO: 1, 2, 3, or 4, (or any aptamer sequence of TABLE 1 or a combination thereof) or a combination thereof. In some aspects, the nucleic acid aptamer further comprises a biologically active molecule covalently or non-covalently attached to the aptamer. In some aspects, the nucleic acid aptamer cross-competes with or binds to the same TLR-4 epitope as a nucleic acid aptamer of SEQ ID NO: 1, 2, 3, or 4 (or any aptamer sequence of TABLE 1 or a combination thereof). In some aspects, the nucleic acid aptamer cross-competes with or binds to an epitope that overlaps the TLR-4 epitope recognized by a nucleic acid aptamer of SEQ ID NO: 1, 2, 3, or 4 (or any aptamer sequence of TABLE 1 or a combination thereof).

In some aspects, the nucleic acid aptamer is administered in a dose regimen comprising multiple doses. In some aspects, the multiple doses are administered concurrently, consecutively, or a combination thereof. In some aspects, the multiple doses comprise two, three, or four, or five doses. In some aspects, each dose comprises between 0.007 and 0.2 mg/kg of nucleic acid aptamer.

In some aspects, the nucleic acid aptamer is administered intravenously, intraarterially, or intraperitoneally. In some aspects, the TLR-4 mediated disease or condition is an ischemic disease or condition. In some aspects, the ischemic condition is myocardial infarction or ischemic stroke. In some aspects, the TLR-4 mediated disease or condition is a hemorrhagic condition. In some aspects, the hemorrhagic condition is hemorrhagic stroke or hemorrhagic transformation. In some aspects, the TLR-4 mediated disease or condition is a neuromuscular disease or condition. In some aspects, the neuromuscular disease or condition is a neurodegenerative disease or condition. In some aspects, the neurodegenerative disease or condition is multiple sclerosis.

### BRIEF DESCRIPTION OF THE DRAWINGS

FIG. 1 shows the primary, secondary, and tertiary sequence of an aptamer of the present disclosure (ApTOLL; SEQ ID NO: 1).

FIG. 2 shows the antagonistic effect of aptamers ApTLR #1R and ApTLR #4F in vitro. HEK-blue cells expressing hTLR-4 coupled to the activation reporter system SEAP were incubated with the selective TLR-4 agonist LPS (200 ng/ml) one hour prior to addition of aptamer (0.2-200 nM) to the incubation medium. hTLR-4 activation was quantified showing a concentration-dependent antagonistic effect of both aptamers in presence of LPS.

FIG. 3 shows the sequence optimization of aptamers ApTLR #1R and ApTLR #4F. Schematic representation showing the elimination of part of the sequence of aptamers ApTLR #1R and ApTLR #4F not involved in the acquisition of the 3D structure, leading to the corresponding truncated forms ApTLR #1RT and ApTLR #4FT.

FIG. 4 shows the confirmation of the maintenance of the hTLR-4 binding capacity of truncated aptamers ApTLR #1RT and ApTLR #4FT. A) Flow cytometry charts depicting the quantification of ApTLR #1RT (red line) and ApTLR #4FT (blue line) to hTLR-4 expressed in 293-hTLRA cells as compared to control HEK293 cells; B) flow cytometry charts showing the slight variation of truncated aptamers binding to hTLR-4 after stimulation of 293-hTLRA cells with LPS.

FIG. 5 shows the confirmation of the antagonistic effect of truncated aptamers ApTLR #1RT and ApTLR #4FT in HEK-blue cells expressing hTLR-4. A) hTLR-4 activation quantified by the reporter system SEAP is shown as compared to the parent aptamers ApTLR #1R and ApTLR #4F. B) Time window of h-TLR-4 activation quantified by SEAP.

FIG. 6 shows the inhibition of hTLR-4 activated by endogenous ligands (DAMPs). hTLR-4 activity assay showing the inhibitory effect of ApTLR #1R, ApTLR #4F and the corresponding truncated forms (0.2-200 nM) against activation mediated by endogenous TLR-4 agonists.

FIG. 7 shows the inhibitory effect of ApTOLL on downstream TLR-4 cell effectors. A) Schematic diagram showing the chemistry underlying the Griess assay for NOx detection; B) quantification of NOx concentration in the medium of peritoneal murine macrophages activated with the TLR-4 agonist LPS and incubated with ApTOLL (20 and 200 nM) one hour later.

FIG. 8 shows the in vitro binding affinity of ApTOLL to TLR-4. Quantification of % of receptor saturation after administration of different concentrations of ApTOLL to Cynomolgus monkey monocytes (A) and human monocytes (B).

FIG. 9 shows the agonistic effect of ApTOLL in other TLRs. TLRs activity assay in HumanTLR2-3-4-5-7-8-and 9 expressing cell lines. No agonistic effect was detected after incubation with ApTOLL (20 and 200 nM).

FIG. 10 shows the hTLR2 and hTLR5 activity assay in HEK-blue-hTLR2 and HEK-blue-hTLR5 cells activated with the hTLR2 and hTLR5 agonists Pam3 and FLAT-ST respectively. Incubation with ApTOLL (20 and 200 nM) showed no inhibitory effect on the activation of hTLR2 and hTLR5 previously activated by the appropriate agonist.

FIG. 11 shows the protective effect of ApTOLL acutely after experimental stroke in mice. A) Quantification of infarct volume in the ApTOLL dose-response study showing protection for the 0.45 mg/kg and 0.9 mg/kg given intraperitoneally (i.p.) 10 minutes after pMCAO; B) quantification of infarct volume in TLR4 knock-out mice showing no effect of ApTOLL. C) Quantification of infarct volume in wild-type animals when ApTOLL is administered intravenously; (*) one-way ANOVA p<0.05 vs vehicle.

FIG. 12 shows the protection mediated by ApTOLL in a permanent middle cerebral artery occlusion by electrocoagulation mouse model. Quantification of infarct volume 24 h after ischemia when 0.91 mg/kg of ApTOLL was administered 10 min after occlusion. (*) t-Student p<0.05 vs vehicle.

FIG. 13 shows the administration of two and three doses (10 min, 2 h and 6 h after ischemia) of ApTOLL in rats after permanent middle cerebral artery occlusion by electrocoagulation. A) Quantification of infarct volume in the ApTOLL multidose study showing protection when 0.45 mg/kg of aptamer is administrated 10 min, 10 min and 2 h, and 10 min-2 h and 6 h after ischemia. (*) one-way ANOVA p<0.05 vs vehicle.

FIG. 14 shows the protection mediated by ApTOLL after ischemia-reperfusion in the rat. A) Quantification showing reduced infarct size at 24 hours after tMCAO in Wistar rats. B) Quantification of infarct volume after ApTOLL or vehicle-treatment in SD rats. (*) t-Student p<0.05 vs vehicle.

FIG. 15 shows a scheme of the design of the therapeutic window of protection of ApTOLL after stroke in mice. Quantification of infarct size at 24 hours after permanent ischemia in mice given ApTOLL at 10 minutes, 2 hours or 6 hours after pMCAO, showing similar extent of protection at all times tested. (*) One-Way ANOVA p<0.05 vs Vehicle.

FIG. 16 shows cytokines determination after ischemia in ApTOLL/vehicle treated animals. Quantification of cytokine levels in plasma 24 h after pMCAO. Results show a significant decrease in some proinflammatory cytokines in plasma from animals after ApTOLL treatment. (*) t-Student p<0.05 vs Vehicle.

FIG. 17 shows long-term anatomical and functional protection induced by acute ApTOLL administration (10 min after occlusion) in mice. A) Quantification on brain edema by T2W-MRI at 24, 48 and 72 hours after stroke showing sustained protection in mice treated with ApTOLL; B) Quantification of infarct size 21 days after stroke on Nissl-stained sections showing long-term protection mediated by acute ApTOLL administration; C-D) Quantification of stride length 21 days after stroke showing absence of neurological deficits in animals treated with ApTOLL acutely; E) Photograph showing a stained path in the footprint test and the different distances that can be potentially altered as a consequence of stroke. (*) Student's t-test p<0.05 vs Vehicle (A, B) or two-way ANOVA p<0.05 vs Sham (C, D); (#) two-way ANOVA p<0.05 vs. MCAO vehicle.

FIG. 18 shows long term motor protection induced by acute administration of ApTOLL (10 min after occlusion) in rats. Assessment of neurological function by the motor score test up to 21 days after pMCAO showed a significant protection at 2 and 7 days after stroke induced by ApTOLL (n=8). Data represent mean±SEM. 2-way ANOVA followed by Bonferroni test (*p<0.05 vs Veh).

FIG. 19 shows the anti-endotoxemic effect of ApTOLL (0.91 mg/kg, 10 min after LPS injection) in a mouse model of sepsis. A) % of weight loss in mice at 8 h and 24 h after intraperitoneal LPS injection (20 mg/kg); B) % of temperature loss in mice at 8 h and 24 h after intraperitoneal injection (20 mg/kg); C) Cumulative sepsis score at 24 hours in mice, showing significant decrease in animals injected with ApTOLL; D) Survival curves up to 72 hours after LPS injection (20 mg/kg), showing increased survival in animals injected with ApTOLL.

FIG. 20 shows the flow chart of the manufacturing process of IMP ApTOLL Drug Product. The IMP was manufactured under full GMP conditions.

FIG. 21 shows the effect of intravenous administration of ApTOLL on physiological parameters. No relevant effect of the administration of aptamer on a battery of physiological parameters measured in blood was observed when compared to intravenous administration of vehicle.

FIG. 22 shows human mixed cortical neurons, cortical glutamatergic neurons and cortical GABAergic neurons with compound treatment. A) Cell Viability (note that 0.01 μM is actually the no treatment control condition (0 μM); used for log graphing purposes only). B) Micrographs from cultures after 10 days of treatment.

FIG. 23 shows the effects of single intravenous administration of ApTOLL on respiratory function in rats. A) Respiratory rate. B) Tidal volume. C) Minute volume.

FIG. 24 shows the binding of aptamers to plasmatic proteins. Elution plots showing fluorescence ApTOLL in fraction bound and unbound to human (A), rat (B) and NHP (C) plasmatic proteins. The grey-shadowed region corresponds to the unbound aptamer peak. The plot shows separately the data for three independent samples.

FIG. 25 shows the detection of ApTOLL in the peripheral and central cells. A) Flow cytometric peripheral analysis of Alexa Fluor 488-labelled ApTOLL (4FT-488; 0.91 mg/kg) in WT and TLR4-KO mice. B) Alexa Fluor 488-labelled ApTOLL in the granulocyte region at 5 minutes after aptamer administration in WT mice. C) Distribution of Alexa Fluor 488-labelled ApTOLL within the brain infarcted region 24 hours after intravenous injection. Pattern of distribution of the aptamer within the ischemic core (green), confirmed by probing with an anti-Alexa-488 antibody conjugated with Cy3 (c; red). D) Unconjugated ApTOLL was used as negative control.

FIG. 26 shows the resistance of ApTOLL to degradation by k-exonuclease A), DNAse I B), and in rat, monkey and human plasma C) at 37° C. A representative gel from 3 experiments is shown.

FIG. 27 shows the histograms of ApTOLL. Incubation with ApTOLL (20 nM) showed no inhibitory effect on the activation of any target selected neither GPCRs, Ion Channels, Kinases, Nuclear Receptors, Transporters nor other Non-Kinase Enzymes. A) Uptake results. B) Binding assays.

FIG. 28 shows the in vitro Absorption. Incubation with ApTOLL (20 nM) showed no inhibitory effect on the transporters selected.

FIG. 29 shows the histogram of ApTOLL. % Inhibition of control values after administration of ApTOLL (20 nM). Results show no significant effects in any inhibition of the CYP enzyme evaluated.

FIG. 30 shows the CYP enzymes induction. Fold induction of vehicle activity after administration of ApTOLL (2-20-200 nM). Cutoff values were predetermined using 10 known CYP inducers and 5 known CYP non-inducers. Results show no significant effects in induction of any CYP enzyme evaluated.

FIG. 31 shows in vitro cytotoxicity assay for ApTOLL. Cell viability assays A) MTT activity and B) LDH determination, quantifying the effect of incubation of HEPG2 and HL60 cell lines with ApTOLL (2-2000 nM) for 24 and 48 hours, showing absence of cytotoxic effects at the biologically active concentrations (2-20 nM). (*) Student's t-test p<0.05 vs. control cells.

FIG. 32 shows the design of the groups involved in the GJ96ND study (Sprague Dawley rat pharmacokinetic study).

FIG. 33 shows summarization of the tₘₐₓ, Cₘₐₓ and AUCₜ values obtained in the MC47KC study (Cynomolgus Monkey toxicity study).

FIG. 34 shows in vitro bacterial cytotoxicity assay ApTOLL. The results for cytotoxicity are expressed as percent of control growth (OD650).

FIG. 35 shows in vitro bacterial cytotoxicity assay ApTOLL in addition to those present in FIG. 34. The results for cytotoxicity are expressed as percent of control growth (OD650).

FIG. 36 shows in vitro Ames test of ApTOLL. Weak positive, if p<0.05, denoted as “+” Strong positive, if p<0.01, denoted as “++” Very strong positive, if p<0.001, denoted as “+++” When possible, compounds which score significantly below background are flagged. This may indicate low level cytotoxicity undetectable by the growth assay. The compounds are flagged as described below. if p<0.05, flagged as “<”, if p<0.01, flagged as “<<”, if p<0.001, flagged as “<<<” Hyphens (−) indicate negative results.

FIG. 37 shows in vitro Ames test results of ApTOLL in addition to those presented in FIG. 36.

FIG. 38 shows in vitro Micronucleus assay of ApTOLL. % of micronuclei cells after ApTOLL treatment at different concentrations. ‘+’ p<0.05 by t-test and % of micronucleated cells at least 3-fold higher than background levels. ‘+/−’ p<0.05 by t-test and % of micronucleated cells at least 2-fold higher than background levels. ‘−’ p>0.05 by t-test and % of micronucleated cells less than 2-fold higher than background levels. CYTO: High cytotoxicity resulting in an insufficient number of scorable cells (>80% cytotoxicity).

FIG. 39 shows A) a scheme of the design of the time window study in rats. Quantification of infarct volume (B) and edema (C) 72 h after transient ischemia in rats when ApTOLL is administered 30 min before reperfusion (B.R.) and 10 min-2 h-6 h-9 h-12 h or 24 h after reperfusion, confirming the protection in tMCAO rats, the extension of the therapeutic window up to 12 h and the protection when ApTOLL is administered before reperfusion.

FIG. 40 shows the effect of ApTOLL in heart's muscular contractibility. The left ventricular echocardiographic parameters A) ejection fraction (%) and B) fractional Shortening (%) were recorded from each rat before (basal) and 72 h after the ischemia-reperfusion myocardial infarction (IR). The treatment was administrated 10 minutes after reperfusion by intravenously injection of Vehicle (PBS with MgCl2, n=7) or a single dose of ApTOLL (0.45 mg/Kg, n=11). (*) Data shown represent mean±SEM. t-Student **p<0.01 vs vehicle.

FIG. 41 shows the results of the preclinical studies in multiple sclerosis. Clinical score during EAE model: disease progression in mice injected with vehicle (n=7) or 0.91 mg/Kg of ApTOLL i.v. (n=12) in the onset of symptoms. (*) Data shown represent mean±SEM. t-Student *p<0.05, **p<0.01, ***p<0.001 vs vehicle.

FIG. 42 shows the effect of ApTOLL in OPCs from rats of 7-days-old. A) The cell survival determined by MTT assay was depicted as % of control (n=3). The H₂O₂ was used as death control. B) The proliferation was quantified by immunocytochemistry and depicted as the % of cells BrdU+/Olig2+ relative to Olig2+ (n=6). C) The differentiation was quantified by immunocytochemistry and depicted as the % of cells MBP+/Olig2+ relative to Olig2+. (n=5). The T3 (Thyroid hormone) was used as differentiation control. (*) Data shown represent mean±SEM. t-Student *p<0.05 vs vehicle.

FIG. 43 shows quantification of infarct volume in an ApTOLL multiple doses study in rats after permanent middle cerebral artery occlusion by electrocoagulation. One (10 min), two (10 min and 2 h), three (10 min, 2 h, and 6 h), four (10 min, 2 h, 6 h, and 24 h) or five (10 min, 2 h, 6 h, 24 h and 48 h) 0.45 mg/kg doses of aptamer were administered after cerebral ischemia. Protection was observed at all dosages tested. All groups were compared with their respective vehicle group (1, 2, 3 and 4 doses were compared with their vehicle groups euthanized at 48 h, and group 5 and its vehicle control group were euthanized at 72 h). (*) t-Test Student, p<0.05 vs Vehicle.

FIG. 44 shows clinical scores in an experimental autoimmune encephalomyelitis (EAE) mouse model when ApTOLL (i.v., 0.91 mg/kg) was administered 24 h after the onset of the symptoms.

FIG. 45 shows the results of ApTOLL in the mice EAE model of MS. Evolution of the clinical score after the administration of different doses of ApTOLL i.v. in independent assays. The number of animals used for each dose was: 0.45 mg/kg dose: 6 EAE-ApTOLL, 15 EAE-VEH and 5 Sham; 0.91 mg/kg dose: 13 EAE-ApTOLL, 6 EAE-VEH and 20 Sham; dose 1.82 mg/kg: 8 EAE-ApTOLL, 7 EAE-VEH and 8 Sham and for the dose 3.6 mg/kg: 5 EAE-ApTOLL, 15 EAE-VEH and 5 Sham. EAE-ApTOLL=EAE model mice treated with ApTOLL. EAE-VEH=EAE model mice treated with vehicle.

FIG. 46 shows a comparison of the four doses of ApTOLL (i.v., 0.45 mg/kg, 0.91 mg/kg, 1.82 mg/kg, and 3.6 mg/kg) studied in the EAE model of MS. The data shows the follow-up of the clinical course of animals treated with each dose of ApTOLL compared to the vehicle group.

FIG. 47 shows the results of a study of myelin loss measured by eriochrome-cyanine staining in spinal cord sections of animals treated with ApTOLL or vehicle at different doses. Quantification of the percentage of demyelination with respect to the area of white matter in each experimental group is represented. ApTOLL induce a decrease in the demyelinated area at all doses studied.

FIG. 48 shows the results of histological studies of remyelination, axonal damage and inflammation comparing EAE-ApTOLL (0.91 and 1.82 mg/kg) and EAE-VEH mice. Graphical representation myelin area (quantification of MBP marker). Graphical representation of the axonal damage area (quantification of NFH marker). Quantification of the percentage of microglia cells with respect to the total cells (Ibal marker).

FIG. 49 shows the quantification of oligodendroglial lineage Olig2+ cell, mature cells (CC1+), and oligodendrocyte precursor cells (PDGFRα), after administration of EAE-ApTOLL (0.91 and 1.82 mg/kg) and EAE-VEH.

FIG. 50 shows cardiac Troponin I (cTnI) levels in plasma at 8- and 24-hours post reperfusion in a model of ischemia/reperfusion myocardial infarct in swine. Values are represented as Mean±SD *p<0.002 ApTOLL 24 h (n=10) vs. VEHICLE 24 h (n=10).

FIG. 51 shows cardiac function of pigs 7 days after reperfusion expressed as EF (Ejection fraction) and FS (Fractional Shortening). N=9 ApTOLL (Aptamer, i.v., 0.078 mg/kg)/8 Placebo (Control). Data is presented as Mean±SD. EF: *p<0.0006 Aptamer vs Control. FS: *p<0.003 Aptamer vs Control.

FIG. 52 shows reduction in infarcted area after double catheterization performed after 7 day of treatment with ApTOLL (i.v., 0.078 mg/kg) or vehicle. A) TTC/Evans Blue double staining was performed in 0.5 cm heart sections, showing the healthy area (marked with a H), the area at risk (R) and the necrotic (infarcted) area (white). B) Quantification of infarcted area expresses as percentage of area at risk. Values are expressed as Mean±SD. *p<0.002 Placebo (Vehicle) vs ApTOLL.

FIG. 53 shows (A) Central panels: bright Field microscopy micrographs (20×) of 0.5 μm heart sections 7 days after reperfusion and H&E stained. External panels: Magnification (60×) of central panels. N=5 ApTOLL/4 Placebo. (B) Bright Field microscopy micrographs of 0.5 μm heart sections 7 days after reperfusion and stained with Masson Trichrome. N=5 ApTOLL/4 Placebo.

FIG. 54 shows confocal microscopy detection of matrix metallopeptidase 9 (MMP-9) in heart sections of pigs treated with ApTOLL or Placebo MMP-9, after 7 days of reperfusion. Nuclei were stained with the fluorescent probe 4′,6-diamidino-2-phenylindole (DAPI). N=5 ApTOLL/4 Placebo. Values are expressed as Mean±SD. *p<0.001 Placebo vs ApTOLL.

FIG. 55 shows the tissue distribution of ApTOLL determined by qPCR (A) Quantification of ApTOLL in heart, lung, kidney, spleen, liver, small intestine, pancreas, thymus and ependymal fat. (B) Quantification of ApTOLL in spleen, kidney and liver. (C) Distribution of ApTOLL in brain in ischemic (ipsilateral and contralateral hemispheres) and naïve rats.

### DETAILED DESCRIPTION

The present disclosure is directed to methods of treatment of TLR-4 mediated diseases and conditions (e.g., myocardial infarction, hemorrhagic stroke, hemorrhagic transformation, ischemic stroke, or multiple sclerosis) comprising administering at least one therapeutically effective dose of at least one nucleic acid aptamer of the present disclosure (e.g., ApTOLL) to a patient in need thereof, alone or in combination with at least another therapy generally used for the treatment of the disease or condition, e.g., pharmacological and/or mechanical thrombolysis (for example, thrombectomy) in myocardial infarction. Also provided are nucleic acid aptamers; chemically modified nucleic acid aptamers; pharmaceutical compositions and formulations comprising aptamers; doses and dosage regimes to practice the methods of the present disclosure; kits and articles of manufacture; and methods of manufacture and formulation.

The diseases and conditions disclosed herein represent a broad sample of TLR-4 mediated diseases and conditions affecting different tissues and organs, having different causes, and having a wide variety of symptom and sequelae, which demonstrates that the nucleic acid aptamers of the present disclosure are broad spectrum medications that can be successfully applied to a wide range of diseases, conditions, as well as their symptoms and sequelae via modulation of TLR-4 mediated cell signaling.

TLR-4 mediated diseases and conditions comprise, e.g., acute diseases and conditions such as enterocolitis, influenza, ischemic stroke, sepsis, renal ischemia-reperfusion, liver ischemia-reperfusion, intracerebral hemorrhage, or myocardial ischemia; sub-acute diseases and conditions such as multiple sclerosis, addiction withdrawal, adenomyosis, keratitis, or pulmonary inflammation; and chronic diseases and conditions such as rheumatoid arthritis, atherosclerosis, asthma, lupus, osteoporosis, transplant rejection, dermatitis, psoriasis, obesity, type II diabetes, neuropathic pain, hypertension, RLA, aortic aneurysm, colon cancer, diffuse axonal injury, or chronic pain. TLR-4 mediated diseases and conditions also comprise, e.g., breast cancer, lung cancer, pancreatic cancer, skin cancer, gastrointestinal cancer, liver cancer, bladder cancer, head and neck cancer, esophageal cancer, gastric cancer, colorectal cancer, ovarian cancer, cervical cancer, or prostate cancer. See, e.g., Mai et al (2013) OncoTargets and Therapy 6: 1573-87, which is herein incorporated by reference in its entirety. Cell migration and invasion in cancer can be reduced by inhibiting TLR-4; accordingly, cancer metastasis can be reduced by inhibiting TLR-4. TLR-4 inhibition can also reduce hepatic steatosis. Accordingly, the methods and compositions disclosed herein can be applied to the treatment of any of the TLR-4 mediated diseases and conditions discloses herein, either alone or in combination with therapeutic interventions (e.g., pharmacological and/or surgical) generally used to treat such TLR-4 mediated diseases and conditions. Furthermore, the methods and compositions disclosed herein can be used to treat symptoms and/or sequelae known in the art related to any of the TLR-4 mediated diseases and conditions disclosed herein and other TLR-4 mediated diseases and conditions known in the art. For example, with respect to use of the methods disclosed herein to treat conditions such a cancer, the disclosed methods and compositions can, e.g., reduce or prevent tumor growth, slow progression, inhibit or reduce angiogenesis, inhibit or reduce tumor invasion, inhibit or reduce metastasis, increase survival, increase quality of life, improve prognosis, etc.

TLR-4 overexpression can contribute to resistance to chemotherapy, e.g., resistance to paclitaxel in ovary cancer and resistance to siRNA therapy in prostate cancer. TLR-4 signaling has also been linked to resistance to chemotherapy in liver cancer. Accordingly, the methods and compositions disclosed herein can be used to reduce, prevent, or reverse resistance to chemotherapy in cancer patients.

TLR-4 signaling in immune and inflammatory cells in a tumor microenvironment lead to the production of inflammatory cytokines, which can result in further polarization of tumor associate macrophages, conversion of fibroblasts into tumor-promoter cancer associated fibroblasts, conversion of dendritic cells into tumor-associated DCs, and activation of pro-tumorigenic function of immature myeloid cells. Accordingly, in some aspects, the methods and compositions of the present disclosure can be used to (i) inhibit or reduce the production of inflammatory cytokines, (ii) reduce or inhibit polarization of tumor associate macrophages, (iii) reduce or inhibit conversion of fibroblasts into tumor-promoter cancer associated fibroblasts, (iv) reduce or inhibit conversion of dendritic cells into tumor-associated DCs, (v) reduce or inhibit activation of pro-tumorigenic function of immature myeloid cells, or (vi) any combination thereof.

Increased TLR-4 activation has been linked to insulin resistance. Thus, with respect to obesity o diabetes, the methods and compositions disclosed herein can be used to reduce or prevent insulin resistance.

Activation of TLR-4 in intrauterine infection lead to uterine smooth muscle contraction. Accordingly, the methods and compositions disclosed herein can be used to prevent or inhibit uterine smooth muscle contraction.

Activation of TLR-4 has also been linked to several autoimmune inflammatory diseases, e.g., human systemic sclerosis (SSc), rheumatoid arthritis, systemic lupus erythematosus, Sjogren's syndrome, psoriasis, multiple sclerosis, or autoimmune diabetes, and it has been observed in particular that inhibition of TLR-4 reduces fibrosis, e.g., dermal or lung fibrosis. Accordingly, the methods and compositions disclosed herein can be used to treat or ameliorate the symptoms of autoimmune inflammatory diseases related to an increased expression and/or activation of TLR-4 such as human systemic sclerosis (SSc), rheumatoid arthritis, systemic lupus erythematosus, Sjogren's syndrome, psoriasis, multiple sclerosis, or autoimmune diabetes. In some aspects, the methods and compositions disclosed herein can be used to inhibit or reduce fibrosis in autoimmune inflammatory diseases related to an increased expression and/or activation of TLR-4 such as human systemic sclerosis (SSc), rheumatoid arthritis, systemic lupus erythematosus, Sjogren's syndrome, psoriasis, multiple sclerosis, or autoimmune diabetes.

In some aspects, the methods and compositions disclosed herein can be used to treat, prevent (e.g., suppress, inhibit or delay), or ameliorate any of the symptoms and sequelae of central nervous diseases including amyotrophic lateral sclerosis (ALS), Parkinson's disease, Huntington's disease, Alzheimer's disease, and vascular dementia disease.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

Accordingly, the terms defined immediately below are more fully defined by reference to the specification in its entirety.

### I. DEFINITIONS

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

About: The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). As used herein, the terms “about” or “at least about” when applied to a series of values or range, apply equally to all member of the list. Accordingly, “at least about 1, 2, 3, 4 . . . ” would be interchangeable with “at least about 1, at least about 2, at least about 3, at least about 4 . . . ”

Administration: The terms “administration,” “administering,” and grammatical variants thereof refer to introducing a composition, such as aptamers of the present disclosure (e.g., ApTOLL), into a subject via a pharmaceutically acceptable route. The introduction of a composition, such as an aptamer of the present disclosure, into a subject can by any suitable route, including orally, pulmonarily, intranasally, parenterally (intravenously, intraarterially, intramuscularly, intraperitoneally, or subcutaneously), rectally, intralymphatically, intrathecally, periocularly or topically. Administration includes self-administration and the administration by another.

A suitable route of administration allows the composition or the aptamer (e.g., ApTOLL) to perform its intended function. For example, if a suitable route is intravenous or intraarterial, the composition is administered by introducing the composition or agent into a vein or artery of the subject.

Antagonist: As used herein, the term “antagonist” refers to a molecule that blocks or dampens an agonist mediated response rather than provoking a biological response itself upon bind to a receptor. Many antagonists achieve their potency by competing with endogenous ligands or substrates at structurally defined binding sites on the receptors. An antagonist can be a competitive, non-competitive, or uncompetitive antagonist. In some aspects of the present disclosure, the antagonist is a TLR-4 antagonist, e.g, an aptamer of the present disclosure such as ApTOLL.

Antibody: As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Aptamer: As used herein, the term “aptamer” refers to a single-stranded nucleic acid chain adopting a specific tertiary structure that allows it to bind to a molecular target with high specificity and affinity, comparable to that of a monoclonal antibody, through interactions other than conventional Watson-Crick base pairing. Generally, aptamers are selected from combinatorial libraries by systemic evolution of ligands by exponential enrichment (SELEX) technology. SELEX is used to identify DNA and RNA aptamers that recognize and selectively bind extra- and intracellular target molecules with high specificity and nanomolar affinity. Once folded under physiological conditions, aptamers acquire unique three-dimensional structures based on their nucleotide sequence, being the tertiary structure of aptamers that confers the selectivity and affinity for their targets.

Aptamer binding site: The term “aptamer binding site” refers to a region in the extracellular regions of TLR-4 comprising a continuous or discontinuous site (i.e., an epitope) to which a complementary aptamer specifically binds. Thus, the aptamer binding site can contain additional areas in the TLR-4 sequence which are beyond the epitope and which can determine properties such as binding affinity and/or stability, or affect properties such as antigen enzymatic activity or dimerization. Accordingly, even if two aptamers bind to the same epitope within the extracellular region of TLR-4, if the aptamers establish distinct intermolecular contacts with amino acids outside of the epitope, such aptamers are considered to bind to distinct aptamer binding sites.

Aptamer of the present disclosure: The term “aptamer of the present disclosure” and grammatical variants thereof refers to an aptamer that can bind to an epitope located on the extracellular domain of TLR-4 and can modulate TLR-4 mediated signaling, e.g., act as a TLR-4 antagonist. In some aspects, the aptamers of the present disclosure prevent or reduce the activation of the NF-kappaB intracellular signaling pathway and/or inflammatory cytokine production. In some aspects, the aptamers of the present disclosure block the inflammatory response released after the onset of a disease or condition disclosed herein (e.g., myocardial infarction, hemorrhagic stroke, hemorrhagic transformation, or multiple sclerosis). In some aspects, the aptamer of the present disclosure is an aptamer of SEQ ID NO: 1-4, or a variant (for example, an aptamer with a certain percentage of sequence identity to an aptamer of SEQ ID NO: 1-4) or derivative thereof (for example, an aptamer of SEQ ID NO: 1-4 or a variant thereof comprising at least one biologically active molecule covalently or non-covalently attached to the aptamer).

In other aspects, the aptamer of the present disclosure is an aptamer that competes with an aptamer of SEQ ID NO: 1-4 for binding to the TLR-4 extracellular domain. In yet another aspect, the aptamer of the present disclosure is an aptamer that binds to an TLR-4 extracellular domain epitope that partially or completely overlaps an epitope to which an aptamer of SEQ ID NO: 1-4 binds. In other aspects, the aptamer of the present disclosure is an aptamer disclosed in TABLE 1 or a variant or derivative thereof.

Binding: The term “binding” refers to a physical interaction between at least two entities, for example, an aptamer and its target epitope, an aptamer and a target protein, or an aptamer and a target cell.

Binding affinity: “Binding affinity” generally refers to the strength of the sum total of non-covalent interactions between a single binding site of a molecule (e.g., an aptamer of the present disclosure) and its binding partner (e.g., TLR-4). Unless indicated otherwise, as used herein, “binding affinity” refers to intrinsic binding affinity which reflects a 1:1 interaction between members of a binding pair (e.g., aptamer and TLR-4). The affinity of a molecule X for its partner Y can generally be represented by its Kₐ (association constant) or its dissociation constant (K_{d}), which is the inverse of the association constant. Affinity can be measured by common methods known in the art, including those described herein. Low-affinity binding molecules, e.g., low-affinity aptamers, generally bind slowly to the target epitope and tend to dissociate readily, whereas high-affinity molecules, e.g., high-affinity aptamers, generally bind to the target epitope faster and tend to remain bound longer. A variety of methods of measuring binding affinity are known in the art, any of which can be used for purposes of the present disclosure.

The ability of an aptamer of the present disclosure (e.g., ApTOLL) to specifically bind to TLR-4 can determined, e.g., by in vitro binding assays, such as the enzyme-linked oligonucleotide assay (ELONA), the enzyme-linked aptamer sorbent assay (ELASA), precipitation and quantitative PCR (qPCR), or by fluorescence techniques such as aptahistochemistry, aptacytochemistry, fluorescence microscopy or flow cytometry. Likewise, both the capability of specific binding to TLR-4 and the affinity of the aptamer for TLR-4 can be determined by techniques well-known by the person skilled in the art, such as gel mobility shift assay, surface plasmon resonance (SPR), kinetic capillary electrophoresis and fluorescence binding assay. Briefly, the fluorescence binding assay consists of the incubation of magnetic balls coated with TLR-4 with different concentrations (for example, from 0 to 100 nM) of the aptamer of the invention labeled (for example, with carboxyfluorescein, FAM), and the subsequent elution and detection of the bound aptamers; the dissociation constants (Kd) are calculated by non-linear fit analysis.

Binding specificity: The terms “specificity” or “binding specificity” refer to the ability of a binding molecule, e.g., an aptamer of the present disclosure, to bind preferentially to an epitope versus a different epitope and does not necessarily imply high affinity. The terms “binding specificity” and “specificity” are used interchangeably and can refer both to (i) a specific portion of a binding molecule (e.g., an aptamer), and (ii) the ability of the binding molecule to specifically bind to a particular epitope. A binding molecule, e.g., an aptamer, “specifically binds” when there is an specific interaction between the aptamer and its target epitope. The term “specifically binds” means that the aptamer has been generated to bind to its target epitope. The term “non-specific binding” means that an aptamer has not been generated to specifically bind to a target epitope but does somehow bind to the epitope through non-specific means.

Biologically active molecule: The term “biologically active molecule” as use herein refers to any molecule that can be attached to an aptamer of the present disclosure (e.g., ApTOLL) covalently or non-covalently, wherein the molecule can have a therapeutic or prophylactic effect in a subject in need thereof, or be used for diagnostic purposes. Accordingly, by way of example, the term biologically active molecule includes proteins (e.g., antibodies, proteins, polypeptides, and derivatives, fragments, and variants thereof), lipids and derivatives thereof, carbohydrates (e.g., glycan portions in glycoproteins), or small molecules. In some aspects, the biologically active molecule is a radioisotope. In some aspects, the biologically active molecule is a detectable moiety, e.g., a radionuclide, a fluorescent molecule, or a contrast agent. In some aspects, a biologically active molecule can be covalently attached to an aptamer of the present disclosure. In some aspects, the biologically active molecule is directly attached to the aptamer. In other aspects, the biologically active molecule is attached to the aptamers via a linker.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of an polynucleotide or polypeptide or can apply to a portion, region or feature thereof.

Cross-compete: The terms “compete” or “cross-compete”, as used herein with regard to a binding molecule, e.g., an aptamer of the present disclosure, means that a first binding molecule, e.g., a first aptamer, binds to an epitope in a manner sufficiently similar to the binding of a second binding molecule, e.g., a second aptamer, such that the result of binding of the first binding molecule with its cognate epitope is detectably decreased in the presence of the second binding molecule compared to the binding of the first binding molecule in the absence of the second binding molecule.

The alternative, where the binding of the second binding molecule to its epitope is also detectably decreased in the presence of the first binding molecule, can, but need not be the case. That is, a first binding molecule can inhibit the binding of a second binding molecule to its epitope without that second molecule inhibiting the binding of the first binding molecule to its respective epitope. However, where each binding molecule detectably inhibits the binding of the other binding molecule with its cognate epitope (or epitopes in the case of a bispecific binding molecule), whether to the same, greater, or lesser extent, the binding molecules are said to “cross-compete” with each other for binding of their respective epitope(s). Both competing and cross-competing binding molecules are encompassed by the present disclosure.

Aptamers are said to “bind to the same epitope” or “comprising the same binding site” or have “essentially the same binding” characteristics, if the aptamers cross-compete so that only one aptamer can bind to the epitope at a given point of time, i.e., one binding molecule prevents the binding or modulating effect of the other.

Competition herein means a greater relative inhibition than at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as determined, e.g., by competition ELONA or ELASA analysis or any suitable method known in the art. It can be desirable to set a higher threshold of relative inhibition as criteria of what is a suitable level of competition in a particular context. Thus, for example, it is possible to set criteria for the competitive binding, wherein at least about 40% relative inhibition is detected, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or even about 100%, before an aptamer is considered sufficiently competitive.

Derived from: The terms “derived from,” “derivative” (e.g., “nucleic acid derivative” or “aptamer derivative”), or any grammatical variant thereof, as used herein, refer to a component that is isolated from or made using a specified molecule (e.g., a nucleic acid aptamer of the present disclosure). For example, a nucleic acid sequence (e.g., aptamer) that is derived from a first nucleic acid sequence (e.g., a parent aptamer) can include a nucleotide sequence that is identical or substantially similar to the nucleotide sequence of the first nucleic acid sequence. In the case of nucleotides, the derived species can be obtained by, for example, naturally occurring mutagenesis, artificial directed mutagenesis or artificial random mutagenesis. The mutagenesis used to derive nucleotides can be intentionally directed or intentionally random, or a mixture of each. The mutagenesis of a nucleotide to create a different nucleotide derived from the first can be a random event (e.g., caused by polymerase infidelity) and the identification of the derived nucleotide can be made by appropriate screening methods.

In some aspects, the derived nucleotide sequences of the present disclosure can be generated, e.g., using combinatorial chemistry, chemically modifying nucleotide units at specific positions, substituting nucleotide units at specific positions with nucleotide analogs, modifying backbone chemical linkages, fusing or conjugating the nucleotide sequence with biologically active molecules, or any combination thereof.

In some aspects, the derived nucleic acid sequence can be generated, e.g., by conjugation to another therapeutic agent (e.g., another TLR antagonist);

(ii) conjugation to a moiety that facilitate targeting (e.g., a ligand, binding moiety, or moiety that directs the aptamer to a certain cell or tissue);

(iii) conjugation to a moiety that modulates, i.e., increases or decreases, plasma half-life (e.g., by modulating resistance to nucleases or altering kidney or liver clearance);

(iv) conjugation to a delivery moiety (e.g., a biopolymer such as PEG or a lipid, peptide, or carbohydrate that would facilitate transport across the blood-brain barrier); or,

(v) any combination thereof.

In some aspects, a nucleotide sequence (e.g., an aptamer) that is derived from a first nucleotide sequence (e.g., a parent aptamer) has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the first nucleotide sequence, respectively, wherein the first nucleotide sequence retains the biological activity of the second nucleotide sequence (in the case of an aptamer of the present disclosure, e.g., the ability to specifically bind to its TLR-4 epitope and to inhibit TLR-4).

Complementary: The terms “complementary” and “complementarity” refer to two or more oligomers (i.e., each comprising a nucleic acid sequence), or between an oligomer and a target gene, that are related with one another by Watson-Crick base-pairing rules. For example, the nucleic acid sequence “T-G-A (5′→3′),” is complementary to the nucleic acid sequence “A-C-T (3′→5′).” Complementarity can be “partial,” in which less than all of the nucleobases of a first nucleic acid sequence are matched to the other nucleobases of a second nucleic acid sequence according to base pairing rules. For example, in some aspects, complementarity between a given nucleic acid sequence and the other nucleic acid sequence can be about 70%, about 75%, about 80%, about 85%, about 90% or about 95%. Or, there can be “complete” or “perfect” (100%) complementarity between a given nucleic acid sequence and the other nucleic acid sequence to continue the example. The degree of complementarity between nucleic acid sequences has significant effects on the efficiency and strength of hybridization between the sequences.

Effective Amount: As used herein, the term “effective amount” of an agent, e.g., an aptamer of the present disclosure (e.g., ApTOLL), is that amount sufficient to effect beneficial or desired results, for example, clinical results, and, as such, an “effective amount” depends upon the context in which it is being applied. For example, in the context of administering an agent that treats a TLR-4 mediated disease or condition, e.g., myocardial infarction, hemorrhagic stroke, hemorrhagic transformation, or multiple sclerosis, or ameliorates or prevents (e.g., suppresses, inhibits or delays) the sequelae and/or symptoms associated with a TLR-4 mediated disease or condition, e.g., myocardial infarction, hemorrhagic stroke, hemorrhagic transformation, or multiple sclerosis, an effective amount of an agent, e.g., an aptamer of the present disclosure is, for example, an amount sufficient to reduce or decrease, e.g., tissue damage, tissue inflammation, physiological, physical, or behavioral symptoms or sequelae, or any combination thereof as compared to the response obtained without administration of the agent.

The term “effective amount” can be used interchangeably with “effective dose,” “therapeutically effective amount,” or “therapeutically effective dose.” In a specific aspect, the term refers to the amount of an aptamer of the present disclosure (e.g., ApTOLL) that can, e.g, treat, prevent, reduce, or ameliorate, a symptom or sequelae of a TLR-4 mediated disease or condition, e.g., myocardial infarction, hemorrhagic stroke, hemorrhagic transformation, or multiple sclerosis

In a particular aspect, the term refers to the amount of an aptamer of the present disclosure (e.g., ApTOLL) needed to achieve (i) reduction in damaged tissue; (ii) reduction in inflammation; (iii) improvement in neurological outcome; (iv) decrease in levels in proinflammatory biomarkers (e.g., interferon-gamma, interleukin-12p70, TNFalpha, IL-6, or any combination thereof); (iv) improvement in motor and/or behavioral scores (e.g., an improvement in mobility or response to stimuli); (v) increase in survival rate; (vi) increase in quality of life; (vii) decrease pain or discomfort, or, (viii) any combination thereof in a subject in need thereof, compared to an untreated subject or to a reference value obtained from a population of untreated subjects.

Epitope: The term “epitope” as used herein refers to a protein determinant (e.g., an amino acid subsequence of TLR-4) capable of binding to a binding molecule, e.g., an aptamer of the present disclosure such as ApTOLL. Epitopes usually consist of chemically active surface groupings of molecules such as amino acids or sugar side chains and usually have specific three dimensional structural characteristics, as well as specific charge characteristics. The part of an aptamer that recognizes the epitope is called a paratope. Epitopes are divided into two categories, conformational epitopes and linear epitopes, based on their structure and interaction with the paratope. A conformational epitope is composed of discontinuous sections of the target protein (e.g., TLR-4) amino acid sequence. These epitopes interact with the aptamer paratope based on the 3-D surface features and shape or tertiary structure of the target protein (e.g., TLR-4). By contrast, linear epitopes interact with the paratope based on their primary structure. A linear epitope is formed by a continuous sequence of amino acids from the target protein (e.g., TLR-4).

Excipient: The terms “excipient” and “carrier” are used interchangeably and refer to an inert substance added to a pharmaceutical composition to further facilitate administration of a compound, e.g., a nucleic acid aptamer of the present disclosure (e.g., ApTOLL).

Homology: As used herein, the term “homology” refers to the overall relatedness between polymeric molecules, e.g. between nucleic acid molecules (e.g. DNA molecules and/or RNA molecules). Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (e.g., polynucleotide sequences).

Identity: As used herein, the term “identity” refers to the overall monomer conservation between polymeric molecules, e.g., between polynucleotide molecules (e.g. DNA molecules and/or RNA molecules). The term “identical” without any additional qualifiers, e.g., nucleic acid A is identical to nucleic acid B, implies the sequences are 100% identical (100% sequence identity). Describing two sequences as, e.g., “70% identical,” is equivalent to describing them as having, e.g., “70% sequence identity.”

Calculation of the percent identity of two polymeric molecules, e.g., polynucleotide sequences, can be performed, for example, by aligning the two sequences for optimal comparison purposes (e.g., gaps can be introduced in one or both of a first and a second polynucleotide sequences for optimal alignment and non-identical sequences can be disregarded for comparison purposes). In certain aspects, the length of a sequence aligned for comparison purposes is at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the length of the reference sequence. The bases at corresponding base positions, in the case of polynucleotides, are then compared.

When a position in the first sequence is occupied by the same base as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence or nucleic acid sequence to a second amino acid sequence or nucleic acid sequence is calculated as % ID=100×(Y/Z), where Y is the number of amino acid residues or nucleobases scored as identical matches in the alignment of the first and second sequences (e.g., as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

Inhibit TLR-4: The terms “inhibit TLR-4,” “inhibition of TLR-4,” “TLR-4 inhibition,” and grammatical variants thereof refer to the blocking and/or reduction of the activation and/or activity of TLR-4, e.g., the transduction of the TLR-4-mediated signal. In the context of the present disclosure, it is considered that TLR-4 is inhibited by an aptamer of the present disclosure (e.g., ApTOLL) if the signaling activity of TLR-4 is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100% compared to the activity of TLR-4 in the present of a natural agonists, e.g., lipopolysaccharide (LPS). Lipopolysaccharide, also known as endotoxin, is a major glycolipid constituent of the outer cell wall of gram-negative bacteria. LPS molecules typically consist of a strain-specific distal polysaccharide side chain known as the O-antigen, a hydrophilic core oligosaccharide, and a hydrophobic domain referred to as lipid A.

In some aspects, the term inhibit TLR-4 refers, e.g., to (i) blockage or complete inhibition of TLR-4 activation, (ii) reduction or partial inhibition of TLR-4 activation, (iii) blockage or complete inhibition of TLR-4 signaling activity, (iv) reduction or partial inhibition of TLR-4 signaling activity, or (v) any combination thereof, by the aptamers of the present disclosure.

The ability of an aptamer of the present disclosure (e.g., ApTOLL) to inhibit TLR-4 can be determined by means of a range of assays that are available in the art. In some aspects, the capability of inhibiting TLR-4 of the aptamer of the present disclosure is determined by means of in vitro assays with cells expressing recombinant TLR-4 and a reporter gene, the expression of which is associated with the activation of recombinant TLR-4. The person skilled in the art will recognize that there are multiple variants of this method, depending on the cell and the recombinant gene used. An example of this assay is included for example, in U.S. Pat. No. 10,196,642, which is herein incorporated by reference in its entirety. Other available techniques include the determination of the levels of inflammatory cytokines, such as IL-1, IL-8, TNF-alpha and IL-12, released by cells that express TLR-4.

Isolated: As used herein, the terms “isolated,” “purified,” “extracted,” and grammatical variants thereof are used interchangeably and refer to the state of a preparation of desired composition of the present disclosure (e.g., an aptamer of the present disclosure), that has undergone one or more processes of purification. In some aspects, isolating or purifying as used herein is the process of removing, partially removing (e.g., a fraction) of a composition of the present disclosure from a sample containing contaminants. In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

Linked: The term “linked” as used herein refers to a first amino acid sequence or polynucleotide sequence (e.g., an aptamer of the present disclosure) covalently or non-covalently joined or attached to a second amino acid sequence or polynucleotide sequence, respectively. The first amino acid or polynucleotide sequence (e.g., an aptamer of the present disclosure) can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

Mismatch: The terms “mismatch” or “mismatches” refer to one or more nucleobases (whether contiguous or separate) in an first nucleic sequence (e.g., an aptamer of the present disclosure) that are not matched to a second nucleic acid sequence (e.g., a variant or derivative of an aptamer of the present disclosure) according to base pairing rules. While perfect complementarity is often desired, some aspects can include one or more but preferably 20, 19, 18, 17, 16, 15, 14, 13, 12, 11, 10, 9, 8, 7, 6, 5, 4, 3, 2, or 1 mismatches between an aptamer variant with respect to the parent aptamer. Variations at any location within the aptamer are included. In certain aspects, aptamers of the present disclosure include variants in nucleobase sequence near the termini, in the interior, and if present are typically within about 20, 19, 18, 17, 16, 15, 14, 13, 12, 11, 10, 9, 8, 7, 6, 5, 4, 3, 2, or 1 subunits of the 5′ and/or 3′ terminus. In certain aspects, 20, 19, 18, 17, 16, 15, 14, 13, 12, 11, 10, 9, 8, 7, 6, 5, 4, 3, 2, or 1 nucleobases can be removed and still provide on-target binding.

Modulate: As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

Nucleic acid: “Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix.

Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure can comprises one or more nucleic acids (e.g., nucleic acid aptamers) as described herein.

The term nucleic acid also encompasses variants such as peptide nucleic acid (PNA), locked nucleic acid (LNA), as well as combinations thereof, modifications thereof, including modified nucleotides, etc. Nucleic acids can be purified from natural sources, produced using recombinant expression systems and, optionally, purified, chemically synthesized, etc. When appropriate, for example, in the case of chemically synthesized molecules, the nucleic acids can comprise nucleoside analogues such as analogues having chemically modified bases or sugars, modifications of the backbone, etc.

Parenteral administration: The phrases “parenteral administration” and “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. In some aspects, parenteral administration is intravenous or intraarterial. In some aspects, intravenous or intraarterial administration is through bolus administration, e.g., through the administration of a slow bolus of a pharmaceutical composition comprising an aptamer of the present disclosure (e.g., ApTOLL).

Pharmaceutically-acceptable carrier: The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

Pharmaceutical composition: As used herein, the term “pharmaceutical composition” refers to one or more of the compounds described herein, such as, e.g., an aptamer of the present disclosure such as ApTOLL, mixed or intermingled with, or suspended in one or more other chemical components, such as pharmaceutically-acceptable carriers and excipients. One purpose of a pharmaceutical composition is to facilitate administration of preparations of aptamer to a subject.

Polynucleotide: The term “polynucleotide” is used interchangeably with “nucleic acid” and refers to polymers of nucleotides of any length, including ribonucleotides, deoxyribonucleotides, analogs thereof, or mixtures thereof. In some aspects, this term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

In some aspects, the term “polynucleotide” includes polydeoxyribonucleotides (containing 2-deoxy-D-ribose), polyribonucleotides (containing D-ribose), including, e.g., double stranded DNA (dsDNA), single stranded DNA (ssDNA), single stranded RNA (ssRNA), or double stranded RNA (dsRNA), whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects, a polynucleotide can be, e.g., a nucleic acid aptamer of the present disclosure (e.g., ApTOLL). In some aspects, the polynucleotide is a DNA. In some aspects, the DNA is a synthetic DNA, e.g., a synthetic ssDNA. In some aspects, the synthetic DNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long. In some aspects, a polypeptide can be covalently or non-covalently attached to an aptamer of the present disclosure.

Prevent: The terms “prevent,” “inhibit,” “suppressing” and variants thereof as used herein applied to a disease or condition disclosed herein, or a symptom or sequela thereof, refer, e.g., to

(i) partially or completely delaying onset of a disease, disorder and/or condition, e.g., any TLR-4 mediated disease or condition disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke);

(ii) partially or completely delaying onset of one or more symptoms, features, or clinical manifestations of a particular disease, disorder, and/or condition, e.g., any TLR-4 mediated disease or condition disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke);

(iii) partially or completely delaying onset of one or more symptoms, features, or manifestations of a particular disease, disorder, and/or condition, e.g., any TLR-4 mediated disease or condition disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke);

(iv) partially or completely delaying progression from a particular disease, disorder and/or condition, e.g., any TLR-4 mediated disease or condition disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke); and/or

(v) decreasing the risk of developing pathology associated with the disease, disorder, and/or condition, e.g., any TLR-4 mediated disease or condition disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke).

In some aspects, preventing, inhibiting, or suppressing an outcome is achieved through prophylactic treatment, e.g., by administering an aptamer of the present disclosure.

Prophylactic: As used herein, “prophylactic” refers to a therapeutic or course of action used to prevent, inhibit, suppress, the onset of a disease or condition, e.g., any TLR-4 mediated disease or condition disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke), or to prevent, inhibit, suppress, or delay a symptom associated with a disease or condition, e.g., any TLR-4 mediated disease or condition disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke).

In some aspects, a prophylactic effect can be achieved by administering an aptamer of the present disclosure, e.g., ApTOLL, to a subject at risk of any TLR-4 mediated disease or condition disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke), or at risk of a certain symptom or sequela after the onset of any TLR-4 mediated disease or condition disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke).

Prophylaxis: As used herein, a “prophylaxis” refers to a measure taken to maintain health and prevent, inhibit, suppress, or delay the onset of a TLR-4 mediated disease or condition disclosed herein, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke, or to prevent, inhibit, suppress, or delay symptoms associated with the occurrence of a TLR-4 mediated disease or condition disclosed herein, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke. In some aspects, the aptamers of the present disclosure can be used for the prophylaxis of a TLR-4 mediated disease or condition disclosed herein, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

Subject: The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

It is to be understood that references to “TLR-4” throughout the present disclosure refer the human TLR-4 with respect to a human subject, and to the respective orthologs when the subject is not a human subject, i.e., the veterinarian application of the methods disclosed herein to, e.g., a horse, cat, or dog subject would require the inhibition of horse, cat or dog TLR-4 by an aptamer of the present disclosure capable of specifically binding to the extracellular domain of horse, cat or dog TLR-4.

Subject in need thereof: As used herein, the phrase “subject in need thereof” includes subjects, such as mammalian subjects, that would benefit from administration of an aptamer of the disclosure, e.g., ApTOLL, e.g., to improve hemostasis.

Susceptible to: A subject who is “susceptible to” or “at risk” of a disease, disorder, and/or condition, or symptoms or sequelae thereof, has not been diagnosed with and/or does not exhibit symptoms of the disease, disorder, and/or condition but harbors a propensity to develop a disease or its symptoms.

In some aspects, a subject who is susceptible or at risk to a disease, disorder, and/or condition (for example, ischemic stroke) can be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition.

In some aspects, a subject who is susceptible or at risk to a disease, disorder, and/or condition will develop the disease, disorder, and/or condition. In some aspects, a subject who is susceptible or at risk to a disease, disorder, and/or condition will not develop the disease, disorder, and/or condition.

Systemic administration: The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, intravenous or intraarterial administration.

Target cell: The term “target cell” as used herein refers to the particular cell that expresses TLR-4, including, inter alia, myeloid lineage cells such as monocytes, macrophages, microglia cells, granulocytes and immature dendritic cells, as well as cells of other lineages such as neurons, etc. In a particular aspect, the target cell is a monocyte or a macrophage. In some aspects, the target cell is a microglia cell. In some aspects, the target cell is a granulocyte. In some aspects, the target cell is an immature dendritic cell. In some aspects, the target cell is a neuron. In some aspects, the aptamers of the present disclosure bind to TLR-4 expressed on the surface of a target cell disclosed herein.

Therapeutically effective amount: As used herein the term “therapeutically effective amount” is the amount of a composition comprising an aptamer of the present disclosure (e.g., ApTOLL) that is sufficient to a produce a desired therapeutic effect, pharmacologic and/or physiologic effect on a subject in need thereof. A therapeutically effective amount can be a “prophylactically effective amount” as prophylaxis can be considered therapy.

The term “therapeutically effective amount” also means an amount of a composition comprising an aptamer of the present disclosure (e.g., ApTOLL) to be delivered that is sufficient to

(i) treat a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(ii) improve symptoms of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(iii) improve sequelae of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(iv) prevent, inhibit, suppress, or delay, a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(v) delay a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(vi) delay the sequelae of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(vii) prevent, inhibit, suppress, or delay, the onset of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(viii) prevent, inhibit, suppress, or delay, the recurrence of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke; or,

(ix) any combination thereof (or any of the actions disclosed below in the definition of the term “treatment”) when administered to a subject
(a) suffering from a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;
(b) susceptible or at risk of having a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;
(c) susceptible or at risk of having a recurrence or exacerbation of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke; or,
(d) at risk of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke due to, for example, an underlying infection, disease, disorder, condition, lifestyle; or,
(e) any combination thereof.

Therapeutically effective outcome: As used herein, the term “therapeutically effective outcome” means an outcome of a treatment (e.g., the administration of at least one dose of an aptamer of the present disclosure, e.g., ApTOLL) that is sufficient in a subject

(i) suffering from a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(ii) susceptible or at risk of having a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(iii) susceptible or at risk of having a recurrence or exacerbation of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;

(iv) at risk of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke due to an underlying infection, disease, disorder, condition, lifestyle; or,

(v) any combination thereof,

to, inter alia, effectively
(a) treat a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;
(b) improve symptoms of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;
(c) improve sequelae of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;
(d) prevent, inhibit, suppress, or delay, a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;
(e) delay a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;
(f) delay the sequelae a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;
(g) prevent, inhibit, suppress, or delay, the onset of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke;
(h) prevent, inhibit, suppress, or delay, the recurrence of a TLR-4 mediated disease or condition, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke; or,
(i) any combination thereof (or any of the actions disclosed below in the definition of the term “treatment”).

TLR-4: The term “TLR-4” as used herein refers to membrane receptor toll-like receptor 4. Activation of TLR-4 produces a signaling cascade resulting, e.g., in the release of inflammatory cytokines such as IL-1, IL-8, TNF-alpha, IL-6, and IL-12, causing inflammation and cell damage. Receptor TLR-4 can also be referred to as ARMD10, CD284, TLR-4 or hTOLL. In humans, receptor TLR-4 was registered in GenBank under accession number 000206.2 on 27 May 2014, and it is encoded by the TLR4 gene. There are several isoforms of TLR-4. The amino acid numbering used to describe the location of the different structural domains in TLR-4 refers to the 839 amino acid long isoform (Isoform 1; Uniprot: 000206-1). Amino acid residues 1-23 constitute the signal sequence, residues 24-631 constitute the extracellular domain, residues 632-652 constitute the transmembrane domain, and residues 653-839 constitute the cytoplasmic domain. TLR-4 Isoform 2 (Uniprot: 000206-2) lacks amino acids 1-40 of the canonical isoform 1 sequence. Accordingly, the extracellular domain of isoform 2 comprises amino acids 41-631 of isoform 1. TLR-4 Isoform 3 (Uniprot: 000206-3) lacks amino acids 1-200 of the canonical isoform 1 sequence. Accordingly, the extracellular domain of isoform 3 comprises amino acids 201-631 of isoform 1.

The term TLR-4 also encompasses polymorphic and natural variants, e.g., allele TLR-4*B (Gly-299, Ile-399) which is associated with a blunted response to inhaled LPS, or natural variants with one or more of the following naturally occurring substitutions: T175A, Q188R, C246S, E287D, D299G, C306W, V310G, N329S, F342Y, L385F, T3991, S400N, F443L, E474K, Q510H, K694R, R763H, or Q834H.

In a particular aspect, an aptamer of the present disclosure binds specifically to an epitope located on the extracellular domain of TLR-4 isoform 1 (i.e., amino acids 24-631 of TLR-4 isoform 1).

In non-human subjects, the term TLR-4 refers to their respective TLR-4s, isoforms, polymorphic forms, and natural variants.

Treatment: The terms “treat,” “treatment,” “therapy,” as used herein refers to, e.g., the reduction in severity of a TLR-4 mediated disease or condition, e.g., myocardial infarction, hemorrhagic stroke, hemorrhagic transformation, multiple sclerosis, or ischemic stroke; the amelioration or elimination of one or more symptoms or sequelae associated with a disease or condition; or the provision of beneficial effects to a subject with a disease or condition, without necessarily curing the disease or condition. The term also includes prophylaxis or prevention (e.g., suppression, inhibition or delay) of a disease or condition or its symptoms or sequelae thereof.

In some aspects, the term refers to a clinical intervention to prevent (e.g., suppress or inhibit) the disease or condition; cure the disease or condition; delay onset of the disease or condition; reduce the seriousness of the disease or condition; improve one or more symptoms; improve one or more sequelae; prevent (e.g., suppress, inhibit or delay) one or more symptoms; prevent (e.g., suppress, inhibit or delay) one or more sequelae; delay one or more symptoms; delay one or more sequelae; ameliorate one or more symptoms; ameliorate one or more sequelae; shorten the duration one or more symptoms; shorten the duration of one or more sequelae; reduce the frequency of one or more symptoms; reduce the frequency of one or more sequelae; reduce the severity of one or more symptoms; reduce the severity of one or more sequelae; improve the quality of life; increase survival; prevent (e.g., suppress, inhibit, or delay) a recurrence of the disease or condition; delay a recurrence of the disease or condition; or any combination thereof, e.g., with respect to what is expected in the absence of the treatment with at least one aptamer of the present disclosure. In some aspects, the disease or conditions is a pathology characterized by an increase in expression of TLR-4 and/or an increase in the activation of TLR-4.

### II. TREATMENT OF TLR-4 MEDIATED DISEASES WITH TLR-4-BINDING APTAMERS

The present disclosure provides methods of treating a TLR-4 mediated disease or condition, e.g., myocardial infarction, hemorrhagic stroke, hemorrhagic transformation, multiple sclerosis, or ischemic stroke in a subject in need thereof comprising administering to the subject at least one therapeutically effective dose of a nucleic acid aptamer from about 40 to about 100 nucleobases in length, e.g., about 40 to about 80 nucleobases in length (e.g., ApTOLL) or a variant or derivative thereof, wherein the aptamer, variant or derivative binds to an epitope on the extracellular domain of TLR-4, and wherein binding of the aptamer to the epitope reduces and/or inhibits TLR-4 activation. In one aspect, the TLR-4 mediated disease or condition is ischemic stroke, or a symptom or sequela thereof. In another aspect, the TLR-4 mediated disease or condition is myocardial infarction, or a symptom or sequela thereof. In yet another aspect, the TLR-4 mediated disease or condition is hemorrhagic stroke, or a symptom or sequela thereof. In some aspects, the TLR-4 mediated disease or condition is hemorrhagic transformation, or a symptom or sequela thereof. In another aspect, the TLR-4 mediated disease or condition is multiple sclerosis, or a symptom or sequela thereof.

It is understood that all the methods disclosed herein can alternatively be formulated as a nucleic acid aptamer from about 40 to about 100 nucleobases in length, e.g., about 40 to about 80 nucleobases in length (e.g., ApTOLL) or a variant or derivative thereof as mentioned above, for use in the treatment of a TLR-4 mediated disease or condition, e.g., myocardial infarction, hemorrhagic stroke, hemorrhagic transformation, multiple sclerosis, or ischemic stroke. Alternatively, also provided is the use of the nucleic acid aptamer for the preparation of a medicament for the treatment of said TLR-4 mediated diseases or conditions.

Also provided are methods to prevent (e.g., suppress, inhibit or delay) at least one symptom or sequela of a TLR-4 mediated disease or condition, e.g., myocardial infarction, hemorrhagic stroke, hemorrhagic transformation, multiple sclerosis, or ischemic stroke in a subject in need thereof comprising administering to the subject at least one therapeutically effective dose of a nucleic acid aptamer from about 40 to about 100 nucleobases in length, e.g., about 40 to about 80 nucleobases in length (e.g., ApTOLL) or a variant or derivative thereof, wherein the aptamer, variant or derivative binds to an epitope on the extracellular domain of TLR-4, and wherein binding of the aptamer to the epitope reduces and/or inhibits TLR-4 activation.

The present disclosure also provides methods to ameliorate at least one symptom of a TLR-4 mediated disease or condition, e.g., myocardial infarction, hemorrhagic stroke, hemorrhagic transformation, multiple sclerosis, or ischemic stroke in a subject in need thereof comprising administering to the subject at least one therapeutically effective dose of a nucleic acid aptamer from about 40 to about 100 nucleobases in length, e.g., about 40 to about 80 nucleobases in length (e.g., ApTOLL) or a variant or derivative thereof, wherein the aptamer, variant or derivative binds to an epitope on the extracellular domain of TLR-4, and wherein binding of the aptamer to the epitope reduces and/or inhibits TLR-4 activation.

The term “ischemic stroke” as used herein refers to a type of stroke (also known as cerebrovascular disease, cerebral infarction, or apoplexy) characterized by a neurological deficit caused by an important decrease in cerebral blood flow in an abnormally abrupt manner. In ischemic stroke, blood irrigation is lost due to the sudden and immediate interruption of blood flow due to occlusion of any of the arteries irrigating the brain mass, which generates the appearance of an infarcted area. Artery occlusion is generally due to atherosclerosis or an embolus (cerebral embolism) that originates in another location, generally the heart or other arteries. Ischemic stroke is a pathology characterized by an increase in the expression of TLR-4 and/or increase in activation of TLR-4. Given that activation of TLR-4 produces a signaling cascade resulting in the release of inflammatory cytokines such as IL-1, IL-8, TNF-alpha, IL-6, and IL-12, causing inflammation and cell damage, the pathology characterized by an increase in expression of TLR-4 and/or an increase in activation of TLR-4 can furthermore be characterized by having an inflammatory component.

In some aspects, ischemic stroke can be thrombotic, embolic, or due to hypoperfusion. In some aspects, ischemic stroke can be caused, for example, by atherosclerosis, vasculitis, vertebral and carotid artery dissection, polycythemia, hypercoagulable state, infection, valvular vegetations, mural thrombi, arterial-arterial emboli from proximal source, fat emboli, septic emboli, cardiac failure resulting in systemic hypotension, sickle cell anemia, compressed blood vessels, ventricular tachycardia, blood clots, cardiorespiratory arrest, stroke, or congenital heart defects. Accordingly, the present disclose provides methods to treat any of these diseases or conditions in a subject in need thereof (for example, a subject suffering from ischemic stroke, at risk of ischemic stroke, or at risk of a recurrence of ischemic stroke) comprising the administering at least one therapeutically effective dose of at least one aptamer of the present disclosure (e.g., ApTOLL) to the subject.

Symptoms and sequelae of ischemic stroke comprise, e.g., unconsciousness, blindness, tonic gaze deviation, global aphasia, dysgraphia, dyslexia, dyscalculia, disorientation, spatial neglect, visual neglect, sensory and/or motor symptoms and deficits in face, sensory and/or motor symptoms in the extremities (upper, lower, or both), urinary incontinence, akinetic mutism, transcortical motor aphasia, confusion, motor hemineglect, hemiparesis, facial plegia, sensory loss, dysarthria, inattention, homonymous hemianopsia, CN deficits, dizziness, vertigo, dystaxia, diplopia, dysphagia, transient ALOC, drop attacks, lightheadedness, quadriplegia, coma, locked-in syndrome, death, Millard-Gubler syndrome, sparing of vertical eye movements, one and a half syndrome, medial inferior pontine syndrome, nystagmus, ataxia, decreased proprioception, medial midpontine syndrome, contralateral paralysis, myoclonus of pharynx/vocal cords/face, lateral superior pontine syndrome, Homer's syndrome, conjugate gaze paresis, loss of pain or temperature in face/extremities/trunk, unilateral headache, visual field defects, visual agnosia, lateral midbrain syndrome, contralateral hemiataxia, tremor, hyperkinesis, medial midbrain syndrome, lateral inferior pontine syndrome, facial paralysis, loss of corneal reflex, hearing loss, limb and gait ataxia, Wallemberg syndrome, hoarseness, clumsy hand syndrome, medial medullary syndrome, tongue deviation, or anterior spinal artery syndrome.

Accordingly, the present disclosure also provides methods to treat, prevent (e.g., suppress, inhibit or delay), or ameliorate any of the symptoms and sequelae of ischemic stroke disclose herein or any combination thereof in a subject in need thereof comprising administering at least one therapeutically effective dose of at least one aptamer of the present disclosure (e.g., ApTOLL) to the subject.

As used herein, the term “hemorrhagic stroke” refers to a condition in which the rupture of a blood vessel of the brain occurs, depriving the area of the brain that depends on that artery of blood. In addition, the blood that flows out compresses brain structures, including other blood vessels, which increases the affected area by ischemia secondary to the intracerebral hemorrhage. Symptoms of hemorrhagic stroke may include total or limited loss of consciousness, nausea, vomiting, sudden and severe headache, weakness or numbness in the face, leg, or arm on one side of the body, seizures, dizziness, loss of balance, problems with speech or swallowing, confusion, or disorientation. The most common cause is an aneurysm. A rarer cause is arteriovenous malformation (AVM). There are two types of hemorrhagic stroke: intracerebral hemorrhage and subarachnoid hemorrhage. In general, the ischemic event caused by the hemorrhagic stroke can cause the sequelae described above for ischemic stroke.

Accordingly, the present disclosure also provides methods to treat, prevent (e.g., suppress, inhibit or delay), or ameliorate any of the symptoms and sequelae of hemorrhagic stroke (either intracerebral hemorrhage or subarachnoid hemorrhage), disclosed herein or any combination thereof in a subject in need thereof comprising administering at least one therapeutically effective dose of at least one aptamer of the present disclosure (e.g., ApTOLL) to the subject.

As used herein, the term “hemorrhagic transformation” refers to the conversion of a bland infarct, e.g., the result of ischemic stroke, into a bloody infarct. Thus, the term refers to bleeding that occurs in dead or dying tissue, e.g., brain tissue deprived of its usual blood supply by an ischemic stroke. The spectrum of hemorrhagic transformation ranges from minor petechial bleeding (hemorrhagic infarct) to major mass-producing hemorrhage (parenchymal hematoma). In general, the hemorrhagic transformation resulting, e.g., from ischemic stroke can cause the sequelae described above for ischemic stroke.

The present disclosure also provides methods to treat, prevent (e.g., suppress, inhibit or delay), or ameliorate any of the symptoms and sequelae of hemorrhagic transformation (e.g., hemorrhagic infarct or parenchymal hematoma), disclosed herein or any combination thereof in a subject in need thereof comprising administering at least one therapeutically effective dose of at least one aptamer of the present disclosure (e.g., ApTOLL) to the subject.

As used herein, the term “myocardial infarction” (also known as “infarction” or “heart attack”), refers to a pathology characterized by insufficient blood supply, with tissue damage, in an area of the heart, caused by an obstruction in one of the coronary arteries. Ischemia or deficient oxygen supply to the heart muscle resulting from such obstruction causes angina pectoris, which if recannulated soon enough, does not cause death of heart tissue, whereas if this anoxia is maintained, the myocardium becomes injured and necrosis, i.e., infarction, ultimately occurs. The cause of myocardial infarction is often atherosclerosis. Other possible causes are coronary artery spasms. A myocardial infarction can cause heart failure, an irregular heartbeat, cardiogenic shock, or cardiac arrest. Risk factor include high blood pressure, smoking, diabetes, lack of exercise, obesity, high blood cholesterol, poor diet, and excessive alcohol intake among others. Impaired blood flood to the cardiac muscle can trigger a ischemic cascade. Myocardial infarction can cause tissue damage (mainly necrosis), resulting in the formation of collagen scars. Tissue death and myocardial scarring alter the normal conduction pathways of the heart and weaken affected areas. Thus, the myocardial infarction can lead to sequelae such as abnormal heart rhythms (arrhythmias), heart block, aneurysm of the heart ventricles, inflammation of the heart, or rupture of the heart.

Thus, the present disclosure also provides methods to treat, prevent (e.g., suppress, inhibit or delay), or ameliorate any of the symptoms and sequelae of myocardial infarction, disclosed herein or any combination thereof in a subject in need thereof comprising administering at least one therapeutically effective dose of at least one aptamer of the present disclosure (e.g., ApTOLL) to the subject.

In some aspects, the administration of an aptamer of the present disclosure (e.g., ApTOLL) to a subject after myocardial infarction results in an improvement in cardiac function of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the cardiac function (as determined, e.g., by measurement of ejection fraction and/or fractional shortening) observed in untreated subjects or in a population of untreated subjects.

As used herein the term “fractional shortening” refers to a measure of the pump function of the heart. It is the ratio between the diameter of the left ventricle when it is relaxed and its diameter when it is contract.

As used herein the term “ejection fraction” refers to the volumetric fraction (or portion of the total) of fluid (usually blood) ejected from a chamber (usually the heart) with each contraction (or heartbeat). Ejection fraction is widely used as a measure of the pumping efficiency of the heart and is used to classify heart failure types. It is also used as an indicator of the severity of heart failure.

The term “multiple sclerosis”, as used herein, refers to a pathology characterized by the onset of demyelinating, neurodegenerative and chronic lesions of the central nervous system. Its causes are currently unknown, although the involvement of various autoimmune mechanisms has been demonstrated. In multiple sclerosis patients, lymphocytes cross the blood-brain barrier to affect the myelin, while an inflammatory process aided by macrophages and neuroglia cells occurs.

Demyelination disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. Multiple sclerosis takes several forms, with new symptoms either occurring in isolated attacks (relapsing forms) or building up over time (progressive forms). Between attacks, symptoms may disappear completely; however, permanent neurological problems often remain, especially as the disease advances.

Multiple sclerosis can cause a variety of symptoms, e.g., changes in sensation (hypoesthesia), muscle weakness, abnormal muscle spasms, or difficulty moving; difficulties with coordination and balance; problems with speech (dysarthria) or swallowing (dysphagia), visual problems (nystagmus, optic neuritis, phosphenes or diplopia), ataxia, tremor, pain, spasms, sexual dysfunction, spasticity, fatigue and acute or chronic pain syndromes, bladder and bowel difficulties, cognitive impairment, or emotional symptomatology (mainly major depression). The main clinical measure of progression of the disability and severity of the symptoms is the Expanded Disability Status Scale or EDDS. Some of the most common cognitive deficits affect recent memory, attention, processing speed, visual-spatial abilities, and executive function.

The main pathophysiological feature of MS, as in other primary demyelinating diseases, is the loss of oligodendrocytes in the central nervous system and, therefore, myelin, both in the white matter and in the gray matter. On the other hand, the autoimmune component that underlies the pathology of multiple sclerosis is the promoter of the processes of inflammation, demyelination and damage to the axonal network, where the TLR-4 and proinflammatory signaling that triggers its activation play a crucial role.

Within the pathophysiological processes that underlie the disease, axonal demyelination of the central and peripheral nervous system plays a crucial role and is the basis of the symptoms presented by individuals affected by the disease. Myelin is a cellular differentiation that allows the correct transmission of the nerve impulse and is physiologically synthesized by oligodendrocytes (in the Central Nerve System) and cells (in the Peripheral Nerve System).

Thus, the present disclosure also provides methods to treat, prevent (e.g., suppress, inhibit or delay), or ameliorate any of the symptoms and sequelae of multiple sclerosis, disclosed herein or any combination thereof in a subject in need thereof comprising administering at least one therapeutically effective dose of at least one aptamer of the present disclosure (e.g., ApTOLL) to the subject.

In some aspects, the administration of an aptamer of the present disclosure (e.g., ApTOLL) to a subject with multiple sclerosis results in a reduction in a clinical score, wherein a higher clinical score relates to a higher degree of disability and severity of the symptoms, and wherein the observed clinical score is less that about 90%, less than about 85%, less than about 80%, less than about 75%, less than about 70%, less than about 65%, less than about 60%, less than about 55%, less than about 50%, less than about 45%, less than about 40%, less than about 35%, or less than about 30% of the clinical score value observed in an untreated subject or in a population of untreated subjects.

In some aspects, the administration of an aptamer of the present disclosure (e.g., ApTOLL) to a subject with multiple sclerosis results in an increase in mobility at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% higher than the increase in mobility observed when the subject is treated with fingolimod (GYLENYA) or methylprednisolone (URBASON®).

In some aspects, the administration of an aptamer of the present disclosure (e.g., ApTOLL) can result in an increase in proliferation of oligodendrocyte precursor cells at least about 5%, at least about 10%, at least about 15%, at least about 20%, at least about 25%, at least about 30%, at least about 35%, at least about 40%, at least about 45%, or at least about 50% higher than the level of proliferation observed in oligodendrocyte precursor cells growing in the absence of aptamer of the present disclosure (e.g., ApTOLL).

In some aspects, the administration of an aptamer of the present disclosure (e.g., ApTOLL) can result in an increase in the differentiation of oligodendrocyte precursor cells at least about 5%, at least about 10%, at least about 15%, at least about 20%, at least about 25%, at least about 30%, at least about 35%, at least about 40%, at least about 45%, or at least about 50% higher than the level of differentiation observed in oligodendrocyte precursor cells growing in the absence of aptamer of the present disclosure (e.g., ApTOLL).

In some aspects, the administration of an aptamer of the present disclosure (e.g., ApTOLL) to a subject in need thereof can result in remyelinization of damaged neuronal tissue, e.g., as a result of an acute (e.g., ischemic stroke, intracerebral hemorrhage, hemorrhagic stroke, or hemorrhagic transformation), sub-acute (e.g., multiple sclerosis), or chronic (e.g., diffuse axonal injury) TLR-4 mediated disease or condition. In some aspects, the administration of an aptamer of the present disclosure (e.g., ApTOLL) to a subject in need thereof can result in neuronal proliferation and/or neuronal differentiation in neuronal tissue damaged during as a result of an acute (e.g., ischemic stroke, intracerebral hemorrhage, or subarachnoid hemorrhage), sub-acute (e.g., multiple sclerosis), or chronic (e.g., diffuse axonal injury) TLR-4 mediated disease or condition. Accordingly, the present disclosure provides a method to remyelinize neuronal tissue damaged as a result of an acute (e.g., ischemic stroke, intracerebral hemorrhage or subarachnoid hemorrhage), sub-acute (e.g., multiple sclerosis), or chronic (e.g., diffuse axonal injury) TLR-4 mediated disease or condition, comprising administering to the subject at least one therapeutically effective dose of a nucleic acid aptamer 40 to 80 nucleobases in length (e.g., ApTOLL) or a variant or derivative thereof, wherein the aptamer, variant or derivative binds to an epitope on the extracellular domain of TLR-4, and wherein binding of the aptamer to the epitope reduces and/or inhibits TLR-4 activation.

In some aspects, the aptamers of the present disclosure (e.g., ApTOLL) are administered less that 16 hours since the onset of a TLR-4 mediated disease or condition, e.g, myocardial infarction, hemorrhagic stroke, hemorrhagic transformation, or ischemic stroke. In some aspects, the aptamers of the present disclosure (e.g., ApTOLL) are administered less than about 5, 10, 15, 20, 25, 30, 35, 40, 45, 50, 55, 60, 65, 70, 75, 80, 95, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, or 960 minutes after the onset of a TLR-4 mediated disease or condition, e.g, myocardial infarction, hemorrhagic stroke, hemorrhagic transformation, or ischemic stroke.

In some aspects, the aptamers of the present disclosure (e.g., ApTOLL) are administered less than about 1 hour, less than about 2 hours, less than about 3 hours, less than about 4 hours, less than about 5 hours, less than about 6 hours, less than about 7 hours, less than about 8 hours, less than about 9 hours, less than about 10 hours, less than about 11 hours, less than about 12 hours, less than about 13 hours, less than about 14 hours, less than about 15 hours, less than about 16 hours, less than about 17 hours, less than about 18 hours, less than about 19 hours, less than about 20 hours, less than about 21 hours, less than about 22 hours, less than about 23 hours, or less than about 24 hours after the onset of the TLR-4 mediated disease or condition, e.g, myocardial infarction, hemorrhagic stroke, hemorrhagic transformation, or ischemic stroke.

In some aspects, the aptamers of the present disclosure (e.g., ApTOLL) are administered about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after the onset of the TLR-4 mediated disease or condition, e.g, myocardial infarction, hemorrhagic stroke, hemorrhagic transformation, or ischemic stroke.

In some aspects, the aptamers of the present disclosure (e.g., ApTOLL) are administered immediately after the onset of a TLR-4 mediated disease or condition, e.g, myocardial infarction, hemorrhagic stroke, hemorrhagic transformation, or ischemic stroke.

In some aspects, additional doses of the aptamers of the present disclosure (e.g., ApTOLL) are subsequently administered after an initial dose. In some aspects, at least 1, 2, 3, 4, 5, 6, 7, 8, 9, or 10 additional doses are administered after the initial dose. In some aspects, several doses are administered during the same day. In some aspects, one or more booster doses are followed by one or more maintenance doses. In some aspects, all the doses comprise the same amount of aptamer of the present disclosure (e.g., ApTOLL).

In some aspects, additional doses of the aptamers of the present disclosure (e.g., ApTOLL) are administered at about 2 hours and about 6 hours after the onset of a TLR-4 mediated disease or condition, e.g, myocardial infarction, hemorrhagic stroke, hemorrhagic transformation, or ischemic stroke. In other aspects, additional doses of the aptamers of the present disclosure (e.g., ApTOLL) are further administered at about 2 hours, about 6 hours, about 12 hours, and about 24 hours after the onset of a TLR-4 mediated disease or condition, e.g, myocardial infarction, hemorrhagic stroke, hemorrhagic transformation, or ischemic stroke.

In some aspects, particularly in the case of acute TLR-4 mediated diseases or conditions, the aptamers of the present disclosure (e.g., ApTOLL) are generally administered minutes (e.g., 10 to 60 minutes), hours (e.g., 1 hour to 48 hours), or days after the acute event. In other aspects, for example in sub-acute (e.g., multiple sclerosis) or chronic (e.g., rheumatoid arthritis) TLR-4 mediated diseases or conditions, the aptamers of the present disclosure (e.g., ApTOLL) can be administered for weeks, months, or years.

In some aspects, the aptamers of the present disclosure (e.g., ApTOLL) are administered at a dosage between about 0.5 mg/day and about 80 mg/day. In some aspects, the aptamers of the present disclosure (e.g., ApTOLL) are administered at a dosage of at least about 0.5 mg/day, at least about 1 mg/day, at least about 2 mg/day, at least about 5 mg/day, at least about 10 mg/day, at least about 15 mg/day, at least about 20 mg/day, at least about 25 mg/day, or at least about 30 mg/day. In some aspects, the aptamers of the present disclosure (e.g., ApTOLL) are administered at a dosage of 0.5 mg/day. In some aspects, the aptamers of the present disclosure (e.g., ApTOLL) are administered at a dosage of 1 mg/day. In some aspects, the aptamers of the present disclosure (e.g., ApTOLL) are administered at a dosage of 2 mg/day. In some aspects, the aptamers of the present disclosure (e.g., ApTOLL) are administered at a dosage of 5 mg/day. In some aspects, the aptamers of the present disclosure (e.g., ApTOLL) are administered at a dosage of 10 mg/day. In some aspects, the aptamers of the present disclosure (e.g., ApTOLL) are administered at a dosage of 15 mg/day. In some aspects, the aptamers of the present disclosure (e.g., ApTOLL) are administered at a dosage of 20 mg/day. In some aspects, the aptamers of the present disclosure (e.g., ApTOLL) are administered at a dosage of 25 mg/day. In some aspects, the aptamers of the present disclosure (e.g., ApTOLL) are administered at a dosage of 30 mg/day.

In some aspects, a dose of approximately 14 mg/kg/day of an aptamer of the present disclosure (e.g., ApTOLL) is considered the No Observed Adverse Effects Level (NOAEL) when the aptamer is administered twice daily (e.g., 6 hours apart) by intravenous or intraarterial route (bolus) for a period of 14 days. In some aspects, the maximum recommended starting dose (MRSD) to be administered to healthy subjects is approximately 31.5 mg for a subject weighing 70 kg. In some aspects, the maximum recommended starting dose (MRSD) to be administered to healthy subjects is approximately 0.5 mg for a subject weighing 70 kg.

In some aspects, the aptamers of the present disclosure (e.g., ApTOLL) are administered at a dose of approximately 0.007 mg/kg (i.e., approx. 0.5 mg/day for a 70 kg subject). In some aspects, the aptamers of the present disclosure (e.g., ApTOLL) are administered at a dosage of at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg, at least about 1.1 mg/kg, at least about 1.2 mg/kg, at least about 1.3 mg/kg, at least about 1.4 mg/kg, at least about 1.5 mg/kg, at least about 1.6 mg/kg, at least about 1.7 mg/kg, at least about 1.8 mg/kg, at least about 1.9 mg/kg, at least about 2 mg/kg, at least about 2.1 mg/kg, at least about 2.2 mg/kg, at least about 2.3 mg/kg, at least about 2.4 mg/kg, at least about 2.5 mg/kg, at least about 2.6 mg/kg, at least about 2.7 mg/kg, at least about 2.8 mg/kg, at least about 2.9 mg/kg, at least about 3 mg/kg, at least about 3.1 mg/kg, at least about 3.2 mg/kg, at least about 3.3 mg/kg, at least about 3.4 mg/kg, at least about 3.5 mg/kg, at least about 3.6 mg/kg, at least about 3.7 mg/kg, at least about 3.8 mg/kg, at least about 3.9 mg/kg, at least about 4 mg/kg, at least about 4.1 mg/kg, at least about 4.2 mg/kg, at least about 4.3 mg/kg, at least about 4.4 mg/kg, at least about 4.5 mg/kg, at least about 4.6 mg/kg, at least about 4.7 mg/kg, at least about 4.8 mg/kg, at least about 4.9 mg/kg, at least about 5 mg/kg, at least about 5.1 mg/kg, at least about 5.2 mg/kg, at least about 5.3 mg/kg, at least about 5.4 mg/kg, at least about 5.5 mg/kg, at least about 5.6 mg/kg, at least about 5.7 mg/kg, at least about 5.8 mg/kg, at least about 5.9 mg/kg, at least about 6 mg/kg, at least about 6.1 mg/kg, at least about 6.2 mg/kg, at least about 6.3 mg/kg, at least about 6.4 mg/kg, at least about 6.5 mg/kg, at least about 6.6 mg/kg, at least about 6.7 mg/kg, at least about 6.8 mg/kg, at least about 6.9 mg/kg, at least about 7 mg/kg, at least about 7.1 mg/kg, at least about 7.2 mg/kg, at least about 7.3 mg/kg, at least about 7.4 mg/kg, at least about 7.5 mg/kg, at least about 7.6 mg/kg, at least about 7.7 mg/kg, about at least 7.8 mg/kg, at least about 7.9 mg/kg, at least about 8 mg/kg, at least about 8.1 mg/kg, at least about 8.2 mg/kg, at least about 8.3 mg/kg, at least about 8.4 mg/kg, at least about 8.5 mg/kg, at least about 8.6 mg/kg, at least about 8.7 mg/kg, at least about 8.8 mg/kg, at least about 8.9 mg/kg, at least about 9 mg/kg, at least about 9.1 mg/kg, at least about 9.2 mg/kg, at least about 9.3 mg/kg, at least about 9.4 mg/kg, at least about 9.5 mg/kg, at least about 9.6 mg/kg, at least about 9.7 mg/kg, at least about 9.8 mg/kg, at least about 9.9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, at least about 20 mg/kg, at least about 21 mg/kg, at least about 22 mg/kg, at least about 23 mg/kg, at least about 24 mg/kg, at least about 25 mg/kg, at least about 26 mg/kg, at least about 27 mg/kg, at least about 28 mg/kg, at least about 29 mg/kg, or at least about 30 mg/kg.

In some aspects, the aptamers of the present disclosure (e.g., ApTOLL) are administered at a dosage of at least about 0.001 mg/kg/day, at least about 0.002 mg/kg/day, at least about 0.003 mg/kg/day, at least about 0.004 mg/kg/day, at least about 0.005 mg/kg/day, at least about 0.006 mg/kg/day, at least about 0.007 mg/kg/day, at least about 0.008 mg/kg/day, at least about 0.009 mg/kg/day, at least about 0.010 mg/kg/day, at least about 0.015 mg/kg/day, at least about 0.020 mg/kg/day, at least about 0.025 mg/kg/day, at least about 0.030 mg/kg/day, at least about 0.035 mg/kg/day, at least about 0.040 mg/kg/day, at least about 0.045 mg/kg/day, at least about 0.050 mg/kg/day, at least about 0.055 mg/kg/day, at least about 0.060 mg/kg/day, at least about 0.065 mg/kg/day, at least about 0.070 mg/kg/day, at least about 0.075 mg/kg/day, at least about 0.080 mg/kg/day, at least about 0.085 mg/kg/day, at least about 0.090 mg/kg/day, at least about 0.095 mg/kg/day, at least about 0.1 mg/kg/day, at least about 0.11 mg/kg/day, at least about 0.12 mg/kg/day, at least about 0.13 mg/kg/day, at least about 0.14 mg/kg/day, or at least about 0.15 mg/kg/day.

In some aspects, the aptamers of the present disclosure (e.g., ApTOLL) are administered at a dosage of at least about 1 μg/kg/day, at least about 1.1 μg/kg/day, at least about 1.2 μg/kg/day, at least about 1.3 μg/kg/day, at least about 1.4 μg/kg/day, at least about 1.5 μg/kg/day, at least about 1.6 μg/kg/day, at least about 1.7 μg/kg/day, at least about 1.8 μg/kg/day, at least about 1.9 μg/kg/day, at least about 2 μg/kg/day, at least about 2.1 μg/kg/day, at least about 2.2 μg/kg/day, at least about 2.3 μg/kg/day, at least about 2.4 μg/kg/day, at least about 2.5 μg/kg/day, at least about 2.6 μg/kg/day, at least about 2.7 μg/kg/day, at least about 2.8 μg/kg/day, at least about 2.9 μg/kg/day, at least about 3 μg/kg/day, at least about 3.1 μg/kg/day, at least about 3.2 μg/kg/day, at least about 3.3 μg/kg/day, at least about 3.4 μg/kg/day, at least about 3.5 μg/kg/day, at least about 3.6 μg/kg/day, at least about 3.7 μg/kg/day, at least about 3.8 μg/kg/day, at least about 3.9 μg/kg/day, at least about 4 μg/kg/day, at least about 4.1 μg/kg/day, at least about 4.2 μg/kg/day, at least about 4.3 μg/kg/day, at least about 4.4 μg/kg/day, at least about 4.5 μg/kg/day, at least about 4.6 μg/kg/day, at least about 4.7 μg/kg/day, at least about 4.8 μg/kg/day, at least about 4.9 μg/kg/day, at least about 5 μg/kg/day, at least about 5.1 μg/kg/day, at least about 5.2 μg/kg/day, at least about 5.3 μg/kg/day, at least about 5.4 μg/kg/day, at least about 5.5 μg/kg/day, at least about 5.6 μg/kg/day, at least about 5.7 μg/kg/day, at least about 5.8 μg/kg/day, at least about 5.9 μg/kg/day, at least about 6 μg/kg/day, at least about 6.1 μg/kg/day, at least about 6.2 μg/kg/day, at least about 6.3 μg/kg/day, at least about 6.4 μg/kg/day, at least about 6.5 μg/kg/day, at least about 6.6 μg/kg/day, at least about 6.7 μg/kg/day, at least about 6.8 μg/kg/day, at least about 6.9 μg/kg/day, at least about 7 μg/kg/day, at least about 7.1 μg/kg/day, at least about 7.2 μg/kg/day, at least about 7.3 μg/kg/day, at least about 7.4 μg/kg/day, at least about 7.5 μg/kg/day, at least about 7.6 μg/kg/day, at least about 7.7 μg/kg/day, at least about 7.8 μg/kg/day, at least about 7.9 μg/kg/day, at least about 8 μg/kg/day, at least about 8.1 μg/kg/day, at least about 8.2 μg/kg/day, at least about 8.3 μg/kg/day, at least about 8.4 μg/kg/day, at least about 8.5 μg/kg/day, at least about 8.6 μg/kg/day, at least about 8.7 μg/kg/day, at least about 8.8 μg/kg/day, at least about 8.9 μg/kg/day, at least about 9 μg/kg/day, at least about 9.1 μg/kg/day, at least about 9.2 μg/kg/day, at least about 9.3 μg/kg/day, at least about 9.4 μg/kg/day, at least about 9.5 μg/kg/day, at least about 9.6 μg/kg/day, at least about 9.7 μg/kg/day, at least about 9.8 μg/kg/day, at least about 9.9 μg/kg/day, at least about 10 μg/kg/day, at least about 10.1 μg/kg/day, at least about 10.2 μg/kg/day, at least about 10.3 μg/kg/day, at least about 10.4 μg/kg/day, at least about 10.5 μg/kg/day, at least about 10.6 μg/kg/day, at least about 10.7 μg/kg/day, at least about 10.8 μg/kg/day, at least about 10.9 μg/kg/day, at least about 11 μg/kg/day, at least about 11.1 μg/kg/day, at least about 11.2 μg/kg/day, at least about 11.3 μg/kg/day, at least about 11.4 μg/kg/day, at least about 11.5 μg/kg/day, at least about 11.6 μg/kg/day, at least about 11.7 μg/kg/day, at least about 11.8 μg/kg/day, at least about 11.9 μg/kg/day, at least about 12 μg/kg/day, at least about 12.1 μg/kg/day, at least about 12.2 μg/kg/day, at least about 12.3 μg/kg/day, at least about 12.4 μg/kg/day, at least about 12.5 μg/kg/day, at least about 12.6 μg/kg/day, at least about 12.7 μg/kg/day, at least about 12.8 μg/kg/day, at least about 12.9 μg/kg/day, at least about 13 μg/kg/day, at least about 13.1 μg/kg/day, at least about 13.2 μg/kg/day, at least about 13.3 μg/kg/day, at least about 13.4 μg/kg/day, at least about 13.5 μg/kg/day, at least about 13.6 μg/kg/day, at least about 13.7 μg/kg/day, at least about 13.8 μg/kg/day, at least about 13.9 μg/kg/day, at least about 14 μg/kg/day, at least about 14.1 μg/kg/day, at least about 14.2 μg/kg/day, at least about 14.3 μg/kg/day, at least about 14.4 μg/kg/day, at least about 14.5 μg/kg/day, at least about 14.6 μg/kg/day, at least about 14.7 μg/kg/day, at least about 14.8 μg/kg/day, at least about 14.9 μg/kg/day, or at least about 15 μg/kg/day.

In some aspects, the aptamers of the present disclosure (e.g., ApTOLL) are administered at a dosage from at least about 1 μg/kg/day to at least about 2 μg/kg/day, from least about 2 μg/kg/day to at least about 3 μg/kg/day, from at least about 3 μg/kg/day to at least about 4 μg/kg/day, from at least about 4 μg/kg/day to at least about 5 μg/kg/day, from at least about 5 μg/kg/day to at least about 6 μg/kg/day, from at least about 6 μg/kg/day to at least about 7 μg/kg/day, from at least about 7 μg/kg/day to at least about 8 μg/kg/day, from at least about 8 μg/kg/day to at least about 9 μg/kg/day, from at least about 9 μg/kg/day to at least about 10 μg/kg/day, from at least about 10 μg/kg/day to at least about 11 μg/kg/day, from at least about 11 μg/kg/day to at least about 12 μg/kg/day, from at least about 12 μg/kg/day to at least about 13 μg/kg/day, from at least about 13 μg/kg/day to at least about 14 μg/kg/day, or from at least about 14 μg/kg/day to at least about 15 μg/kg/day.

In some aspects, the aptamers of the present disclosure (e.g., ApTOLL) are administered at a dosage from at least about 1 μg/kg/day to at least about 3 μg/kg/day, from at least about 3 μg/kg/day to at least about 6 μg/kg/day, from at least about 6 μg/kg/day to at least about 9 μg/kg/day, from at least about 9 μg/kg/day to at least about 12 μg/kg/day, or from at least about 12 μg/kg/day to at least about 15 μg/kg/day.

In some aspects, the aptamers of the present disclosure (e.g., ApTOLL) are administered at a dosage from at least about 1 μg/kg/day to at least about 4 μg/kg/day, from at least about 4 μg/kg/day to at least about 8 μg/kg/day, from at least about 8 μg/kg/day to at least about 12 μg/kg/day, from at least about 11 μg/kg/day to at least about 15 μg/kg/day.

In some aspects, the aptamers of the present disclosure (e.g., ApTOLL) are administered at a dosage from at least about 1 μg/kg/day to at least about 5 μg/kg/day, from at least about 5 μg/kg/day to at least about 10 μg/kg/day, or from at least about 10 μg/kg/day to at least about 15 μg/kg/day.

In some aspects, the aptamers of the present disclosure (e.g., ApTOLL) are administered at a dosage from at least about 6.5 μg/kg/day to at least about 7.5 μg/kg/day, from at least about 6 μg/kg/day to at least about 8 μg/kg/day, from at least about 5.5 μg/kg/day to at least about 8.5 μg/kg/day, from at least about 5 μg/kg/day to at least about 9 μg/kg/day, from at least about 4.5 μg/kg/day to at least about 9.5 μg/kg/day, from at least about 4 μg/kg/day to at least about 10 μg/kg/day, from at least about 3.5 μg/kg/day to at least about 10.5 μg/kg/day, from at least about 3 μg/kg/day to at least about 11 μg/kg/day, from at least about 2.5 μg/kg/day to at least about 11.5 μg/kg/day, from at least about 2 μg/kg/day to at least about 12 μg/kg/day, from at least about 1.5 μg/kg/day to at least about 12.5 μg/kg/day, from at least about 1 μg/kg/day to at least about 13 μg/kg/day, from at least about 1 μg/kg/day to at least about 13.5 μg/kg/day, from at least about 1 μg/kg/day to at least about 14 μg/kg/day, from at least about 1 μg/kg/day to at least about 14.5 μg/kg/day, or from at least about 1 μg/kg/day to at least about 15 μg/kg/day

The dosages disclosed above can be administered as a single dose or multiple doses during a day. Accordingly, a total daily dose of 0.6 mg can be administered, e.g., as two 0.3 mg doses, or three 0.2 mg doses, or five 0.1 doses.

In some aspects, the aptamer of the present disclosure (e.g., ApTOLL) has a T_{1/2} (blood plasma half-life) of about 0.5 hours, about 0.6 hours, about 0.7 hours, about 0.8 hours, about 0.9 hours, about 1 hour, about 1.1 hours, about 1.2 hours, about 1.3 hours, about 1.4 hours, about 1.5 hours, about 1.6 hours, about 1.7 hours, about 1.8 hours, about 1.9 hours, about 2 hours, about 2.1 hours, about 2.2 hours, about 2.3 hours, about 2.4 hours, about 2.5 hours, about 2.6 hours, about 2.7 hours, about 2.8 hours, about 2.9 hours, about 3 hours, about 4 hours, about 5 hours, about 6 hours, about 7 hours, or about 8 hours. In one specific aspect, the T_{1/2} of the aptamer (e.g., ApTOLL) is about 0.8 and 1.4 hours. In one specific aspect, the T_{1/2} of the aptamer (e.g., ApTOLL) is about 1.4 hours. In one specific aspect, the T_{1/2} of ApTOLL in human plasma is about 8 hours.

In some aspects, the aptamers of the present disclosure (e.g., ApTOLL) are administered in multiple doses. In one aspect, the aptamers are administered in one, two, three, four, five, six, seven, eight, nine or ten doses. In some aspects, the aptamers are administered in three doses. In some aspects, the three doses are administered during the same day. In some aspects, a first dose is administered less than an hour after the onset of TLR-4 mediated disease or condition, e.g., myocardial infarction, hemorrhagic stroke, hemorrhagic transformation, or ischemic stroke, e.g., 10 minutes after the onset of the TLR-4 mediated disease or condition, e.g., myocardial infarction, hemorrhagic stroke, hemorrhagic transformation, or ischemic stroke. In some aspects, a second dose is administered less than 3 hours after the onset of the TLR-4 mediated disease or condition, e.g., myocardial infarction, hemorrhagic stroke, hemorrhagic transformation, or ischemic stroke, e.g., about 2 hours after the onset of the TLR-4 mediated disease or condition, e.g., myocardial infarction, hemorrhagic stroke, hemorrhagic transformation, or ischemic stroke. In some aspects, a third dose is administered less than 8 hours after the onset of the TLR-4 mediated disease or condition, e.g., myocardial infarction, hemorrhagic stroke, hemorrhagic transformation, or ischemic stroke, e.g., about 6 hours after the onset of the TLR-4 mediated disease or condition, e.g., myocardial infarction, hemorrhagic stroke, hemorrhagic transformation, or ischemic stroke.

In some aspects, the aptamers of the present disclosure (e.g., ApTOLL) are administered intravenously or intraarterially. In a particular aspect, the aptamers of the present disclosure are administered as a bolus. In some aspects, the bolus is a slow bolus.

In some aspects, the administration of the aptamers of the present disclosure (e.g., ApTOLL) to a subject having a TLR-4 mediated disease or condition, e.g., myocardial infarction, hemorrhagic stroke, hemorrhagic transformation, ischemic stroke, multiple sclerosis, results in

(i) reduction in damaged tissue;

(ii) reduction in inflammation;

(iii) improvement in prognosis and outcome;

(iv) decrease in levels in proinflammatory biomarkers (e.g., interferon-gamma, interleukin-12p70, TNFalpha, IL-6, or any combination thereof);

(v) increase in quality of life;

(vi) improvement in functional scores, e.g., motor score (e.g., an improvement in mobility);

(vii) increase in survival; or,

(v) any combination thereof.

The effects described above are with respect to a control subject or a population of control subjects that has or has had the TLR-4 mediated disease or condition, e.g., myocardial infarction, hemorrhagic stroke, hemorrhagic transformation, ischemic stroke, multiple sclerosis but have not been administered an aptamer of the present disclosure, e.g., ApTOLL.

In some aspects, the administration of an aptamer of the present disclosure (e.g., ApTOLL) to a subject after the onset of a TLR-4 mediated disease or condition, e.g., myocardial infarction, hemorrhagic stroke, hemorrhagic transformation, ischemic stroke, or multiple sclerosis, results in a reduction in tissue damage (e.g., brain tissue or heart tissue) between 20% and 75% with respect to an untreated subject or a reference value obtained from a control population of untreated subjects. In one specific aspects, the administration of an aptamer of the present disclosure (e.g., ApTOLL) to a subject after the onset of a TLR-4 mediated disease or condition, e.g., myocardial infarction, hemorrhagic stroke, hemorrhagic transformation, ischemic stroke, or multiple sclerosis, results in a reduction in tissue damage (e.g., brain tissue or heart tissue) of approximately 65% with respect to an untreated subject or a reference value obtained from a control population of untreated subjects.

In some aspect, the administration of an aptamer of the present disclosure to a subject after the onset of a TLR-4 mediated disease or condition, e.g., myocardial infarction, hemorrhagic stroke, hemorrhagic transformation, ischemic stroke, or multiple sclerosis, results in a reduction in tissue damage (e.g., brain tissue or heart tissue) of at least about 10%, at least about 15%, at least about 20%, at least about 25%, at least about 30%, at least about 35%, at least about 40%, at least about 45%, at least about 50%, at least about 55%, at least about 55%, at least about 60%, at least about 65%, at least about 70%, or at least about 75% with respect to an untreated subject or a reference value obtained from a control population of untreated subjects.

In a specific aspect, the administration of the aptamers of the present disclosure (e.g., ApTOLL) to a subject in need thereof causes a reduction in the size of a damaged or lesioned area (e.g., infarcted area after an ischemic event) that is significantly smaller when the administration is in a multidose regimen. For example, in a specific aspect, the administration of three doses of the aptamer (e.g., at 10 minutes, 2 hours, and 6 hours after infarction) reduces size of the damaged or lesioned area (e.g., infarcted area after an ischemic event) by at least 24%, compared to a reduction of approximately 19% observed when a single dose is administered at 10 minutes after infarction.

In some aspects, the administration of a multidose regimen of the aptamers of the presented disclosure (e.g., ApTOLL) to a subject in need thereof results in an efficacy in the reduction of the size of a damaged or lesioned area (e.g., infarcted area after an ischemic event) of at least at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, at least about 100%, at least 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, at least about 200%, at least about 205%, at least about 210%, at least about 215%, at least about 220%, at least about 225%, at least about 230%, at least about 235%, at least about 240%, at least about 245%, at least about 250%, at least about 255%, at least about 260%, at least about 265%, at least about 270%, at least about 275%, at least about 280%, at least about 285%, at least about 290%, at least about 295%, at least about 300%, at least about 305%, at least about 310%, at least about 315%, at least about 320%, at least about 325%, at least about 330%, at least about 335%, at least about 340%, at least about 345%, or at least about 350% compared to the size of the damaged or lesioned area (e.g., infarcted area after an ischemic event) observed after administration of a corresponding single dose regimen.

In some aspects, the treatment of a TLR-4 mediated disclosed or condition disclosed herein, e.g., e.g., myocardial infarction, hemorrhagic stroke, hemorrhagic transformation, ischemic stroke, or multiple sclerosis, by administering at least one aptamer of the present disclosure (e.g., ApTOLL) can be combined with other therapeutic and/or prophylactic treatments. For example, aptamers of the present disclosure can be administered with biologically active molecules such as anticoagulants, anti-inflammatories, or blood pressure regulators.

In some aspects, the administration of the aptamers of the present disclosure (e.g., ApTOLL) can be combined, for example, with a surgical intervention, e.g., thrombectomy) in subject suffering a myocardial infarction, hemorrhagic stroke, hemorrhagic transformation, or ischemic stroke. In some aspects, the administration of aptamers of the present disclose can be combined with catheterization, e.g., balloon catheterization, or the insertion of a stent. In some aspects, artery recanalization can be induced pharmacologically (e.g., thrombolysis), mechanically (e.g., endovascular thrombectomy), or a combination thereof.

In some aspects, the administration of the aptamers of the present disclosure (e.g., ApTOLL) takes place before, during, or after surgery (e.g., thrombectomy), or a combination thereof. In some aspects, administration of the aptamers of the present disclosure (e.g., ApTOLL) takes place before, during, or after thrombolysis, e.g., pharmacological thrombolysis, pharmacomechanical thrombolysis, mechanical thrombectomy, or a combination thereof. In some aspects, the thrombectomy is stent-retriever thrombectomy, balloon embolectomy, direct aspiration thrombectomy, surgical embolectomy, or any combination thereof.

In some aspects, the methods of treatment of ischemic stroke disclosed herein comprise thrombolysis (e.g., pharmacomechanical thrombolysis) and/or thrombectomy (e.g., mechanical thrombectomy) combined with the administration of aptamers of the present disclosure (e.g., ApTOLL), wherein the combined treatment results in an increase in efficacy in the reduction in tissue damage (e.g., reduction of infarcted area) of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, at least about 200%, at least about 205%, at least about 210%, at least about 215%, at least about 220%, at least about 225%, at least about 230%, at least about 235%, at least about 240%, at least about 245%, at least about 250%, at least about 255%, at least about 260%, at least about 265%, at least about 270%, at least about 275%, at least about 280%, at least about 285%, at least about 290%, at least about 295%, at least about 300%, at least about 305%, at least about 310%, at least about 315%, at least about 320%, at least about 325%, at least about 330%, at least about 335%, at least about 340%, at least about 345%, or at least about 350% compared to the efficacy in the reduction of the tissue damage (e.g., infarcted area) observed following the administration of an aptamer of the present disclosure (e.g., ApTOLL) in the absence of thrombolysis.

In some aspects of the present disclosure, the administration of the aptamers of the present disclosure (e.g., ApTOLL) to a subject after the onset of a TLR-4 mediated disease or condition, e.g., myocardial infarction, hemorrhagic stroke, hemorrhagic transformation, ischemic stroke, or multiple sclerosis, results in a protective effect.

Accordingly, in some aspects, the administration of the aptamers of the present disclosure (e.g., ApTOLL) to a subject after the onset of a TLR-4 mediated disease or condition, e.g., ischemic stroke, results in sustained reduction in incidence of particular complication, e.g., brain infarction, for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the onset of a TLR-4 mediated disease or condition.

In some aspects, the administration of the aptamers of the present disclosure (e.g., ApTOLL) to a subject the onset of a TLR-4 mediated disease or condition, e.g., ischemic stroke, results in sustained reduction in incidence of particular complication, e.g., brain infarction, for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the administration of an aptamer of the present disclosure (alone or in combination with pharmacological, e.g., thrombolysis, and/or mechanical, e.g., endovascular thrombectomy, interventions).

In some aspects, the administration of the aptamers of the present disclosure (e.g., ApTOLL) to a subject after the onset of a TLR-4 mediated disease or condition, such as myocardial infarction, hemorrhagic stroke, hemorrhagic transformation, or ischemic stroke, event results in a sustained protective effect (e.g., reduction in recurrence, reduction tissue damage, reduction in inflammation, reduction in symptoms and/or sequelae, or any combination thereof) for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the onset of a TLR-4 mediated disease or condition.

In some aspects, the administration of the aptamers of the present disclosure (e.g., ApTOLL) to a subject after the onset of a TLR-4 mediated disease or condition such as myocardial infarction, hemorrhagic stroke, hemorrhagic transformation, or ischemic stroke results in a sustained protective effect (e.g., reduction in recurrence, reduction tissue damage, reduction in inflammation, reduction in symptoms and/or sequelae, or any combination thereof) for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the administration of an aptamer of the present disclosure (alone or in combination with pharmacological, e.g., thrombolysis, and/or mechanical, e.g., endovascular thrombectomy, interventions).

In some aspects, the administration of the aptamers of the present disclosure (e.g., ApTOLL) to a subject after the onset of a TLR-4 mediated disease or condition such as myocardial infarction, hemorrhagic stroke, hemorrhagic transformation, or ischemic stroke results in a sustained protective effect (e.g., reduction in recurrence, reduction tissue damage, reduction in inflammation, reduction in symptoms and/or sequelae, or any combination thereof) for at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, or at least about 28 days after the onset of the TLR-4 mediated disease or condition.

In some aspects, the administration of the aptamers of the present disclosure (e.g., ApTOLL) to a subject after the onset of a TLR-4 mediated disease or condition such as myocardial infarction, hemorrhagic stroke, hemorrhagic transformation, or ischemic stroke results in a sustained protective effect (e.g., reduction in recurrence, reduction tissue damage, reduction in inflammation, reduction in symptoms and/or sequelae, or any combination thereof) for at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, or at least about 28 days after the administration of an aptamer of the present disclosure (alone or in combination with pharmacological, e.g., thrombolysis, and/or mechanical, e.g., endovascular thrombectomy, interventions).

In some aspects, the administration of the aptamers of the present disclosure (e.g., ApTOLL) to a subject after the onset of a TLR-4 mediated disease or condition such as myocardial infarction, hemorrhagic stroke, hemorrhagic transformation, ischemic stroke, or multiple sclerosis results in a reduction of the volume of damaged tissue (e.g., infarct volume) (e.g., as determined after 24 hours, 48 hours, or 72 hours after the onset of the TLR-4 mediated disease or condition) of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% with respect to volume of damaged tissue observed in control subjects or in a control population in the absence of treatment with the aptamers of the present disclosure.

In some aspects, the administration of the aptamers of the present disclosure (e.g., ApTOLL) to a subject after the onset of a TLR-4 mediated disease or condition such as myocardial infarction, hemorrhagic stroke, hemorrhagic transformation, ischemic stroke, or multiple sclerosis results in a reduction of the volume of damaged tissue (e.g., infarct volume) (e.g., as determined after 24 hours, 48 hours, or 72 hours after the onset of the TLR-4 mediated disease or condition) of about 10%, about 15%, about 20%, about 25%, about 30%, about 35%, about 40%, about 45%, about 50%, about 55%, about 60%, about 65%, about 70%, or about 75% with respect to volume of damaged tissue observed in control subjects or in a control population in the absence of treatment with the aptamers of the present disclosure.

In some aspects, the administration of the aptamers of the present disclosure (e.g., ApTOLL) to a subject after the onset of a TLR-4 mediated disease or condition such as myocardial infarction, hemorrhagic stroke, hemorrhagic transformation, ischemic stroke, or multiple sclerosis results in a reduction of tissue injury (e.g., cortex injury or heart muscle injury) of at least about 10%, at least about 15%, at least about 20%, at least about 25%, at least about 30%, at least about 35%, or at least about 40% with respect to tissue injury (e.g., cortex injury or heart muscle injury) observed in control subjects or in a control population in the absence of treatment with the aptamers of the present disclosure.

In some aspects, the administration of the aptamers of the present disclosure (e.g., ApTOLL) to a subject after the onset of a TLR-4 mediated disease or condition, e.g., myocardial infarction, hemorrhagic stroke, hemorrhagic transformation, ischemic stroke, or multiple sclerosis, results in an improvement in neurological recovery of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, with respect to the neurological recovery observed in control subjects or in a control population in the absence of treatment with the aptamers of the present disclosure.

In some aspects, the administration of the aptamers of the present disclosure (e.g., ApTOLL) to a subject after the onset of a TLR-4 mediated disease or condition, e.g., myocardial infarction, hemorrhagic stroke, hemorrhagic transformation, ischemic stroke, or multiple sclerosis, in an improvement in motor function of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, with respect to the motor scores observed in control subjects or in a control population in the absence of treatment with the aptamers of the present disclosure.

In some aspects, the administration of the aptamers of the present disclosure (e.g., ApTOLL) to a subject after the onset of a TLR-4 mediated disease or condition, e.g., myocardial infarction, hemorrhagic stroke, hemorrhagic transformation, ischemic stroke, or multiple sclerosis, results in a reduction in the plasma protein levels of pro-inflammatory biomarkers of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, with respect to the plasma protein levels of pro-inflammatory biomarkers observed in control subjects or in a control population in the absence of treatment with the aptamers of the present disclosure.

In some aspects, the aptamers of the present disclosure (e.g., ApTOLL) can be administered via intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. In specific aspects, the aptamers of the present disclosure (e.g., ApTOLL) are administered intravenously or intraarterially, e.g., via infusion or via bolus. In some aspects, the administration is via a slow bolus, i.e., the dose is administered via injection lasting about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, or about 15 minutes.

In some aspects, the aptamers of the present disclosure (e.g., ApTOLL) can be used concurrently with other medicaments or treatments suitable for the treatment of ischemic conditions and/or thrombi, e.g., thrombolysis as discussed above.

In some aspects, the use aptamers of the present disclosure (e.g., ApTOLL) according to the methods disclosed herein can be combined with one or more therapies (pharmacological and/or surgical) for the treatment of ischemic stroke known in the art. In some aspects, the use aptamers of the present disclosure (e.g., ApTOLL) according to the methods disclosed herein can be combined with one or more therapies (pharmacological and/or surgical) for the treatment of myocardial infarction known in the art. In some aspects, the use aptamers of the present disclosure (e.g., ApTOLL) according to the methods disclosed herein can be combined with one or more therapies (pharmacological and/or surgical) for the treatment of myocardial infarction known in the art. In some aspects, the use aptamers of the present disclosure (e.g., ApTOLL) according to the methods disclosed herein can be combined with one or more therapies (pharmacological and/or surgical) for the treatment of hemorrhagic stroke known in the art. In some aspects, the use aptamers of the present disclosure (e.g., ApTOLL) according to the methods disclosed herein can be combined with one or more therapies (pharmacological and/or surgical) for the treatment of hemorrhagic transformation known in the art. In some aspects, the use aptamers of the present disclosure (e.g., ApTOLL) according to the methods disclosed herein can be combined with one or more therapies (pharmacological and/or surgical) for the treatment of multiple sclerosis known in the art.

In some aspects, the aptamers of the present disclosure (e.g., ApTOLL) can be administered in combination of, e.g., a TLR-4 antagonist, an anti-inflammatory agent, a nucleic acid, a peptide or protein, or a combination thereof. In some aspects, the methods disclosed herein can also be combined with operative procedures such a carotid endarterectomy and/or carotid stenting.

In some aspects, the methods disclosed herein comprise the administration of at least one aptamer of the present disclosure (e.g., ApTOLL), alone or in combination with pharmacological or mechanicals thrombolysis, and optionally in combination with ibudilast, TAK242, NI-0101, eritoran, edaravone, uric acid, fingolimod, natalizumab, minocycline, anakinra, nerinetide, or any combination thereof.

In some aspects, the methods disclosed herein comprise the co-administration of at least one of the aptamers of the present disclosure (e.g., ApTOLL) as a combination therapy comprising the administration of

(i) a TLR-4 antagonist selected from the group consisting of naloxone, (+)-naloxone, naltrexone, (+)-naltrexone, lipopolysaccharide (LPS), ibudilast, propentofylline, amitriptyline, ketotifen, cyclobenzaprine, mianserin, imipramine, a lipid A analog (e.g., eritoran or E5531), pinocembrin, palmitoylethanolamide, tapentadol, polypropyletherimine dendrimer glucosamine (DG), aminoalkyl glucosaminide 4-phosphate (e.g., CRX-526), IAXO-102, Rs-LPS, TLR-IN-C34, TAK-242, E5564, or any combination thereof;

(ii) an anti-platelet drug, e.g., aspirin or clopidogrel;

(iii) an anti-coagulant, e.g., heparin, acenocumarol, warfarin, dabigatran, or rivaroxaban;

(iv) an antioxidant, e.g., edaravone;

(v) tissue plasminogen activator, or,

(vi) any combination thereof.

In some aspects, the methods disclosed herein comprise the co-administration of at least one of the aptamers of the present disclosure (e.g., ApTOLL) as a combination therapy comprising the administration of nucleic acids which have the capability of silencing the expression of genes involved in a pathology characterized by an increase in expression of TLR-4 and/or an increase in activation of TLR-4, e.g., antisense oligonucleotides (e.g., antisense RNA, antisense DNA, or antisense RNA/DNA), small interfering RNA (siRNA), short hairpin RNA (shRNA), anti microRNA (antimir); peptides, such as signaling peptides and target-binding peptides (e.g., antibodies or antigen binding fragment thereof, of compounds comprising antibodies or antigen binding fragments thereof such as antigen-drug conjugates or immunotoxins).

In some aspects, the methods disclosed herein comprise the administration of at least one aptamer of the present disclosure, e.g., ApTOLL or any of the aptamer disclosed below, particularly, any of the aptamers disclosed in TABLE 1 or a variant or derivative thereof.

In some aspects, the methods disclosed herein can be practiced using nucleic acids other than aptamers that, instead of reducing and/or inhibiting TLR-4 action by binding to the TLR-4 protein, reduce and/or inhibit (e.g., deplete or abolish) TLR-4 expression directly or indirectly, by interacting with the TLR4 gene or transcription products of the TLR4 gene such as messenger RNA (mRNA) encoding TLR-4, or with nucleic acids modulating the expression of TLR-4 (e.g., miRNA) for example, antisense oligonucleotides, siRNAs, shRNAs, or antimirs. Also contemplated is the practice of the methods disclosed here using agents that transiently or permanently alter TLR-4 expression, e.g., gene therapy approaches using, for example, CRISPR/Cas, TALEN, or ZFN. Also contemplated is the practice of the methods disclosed herein using agents that post-transcriptionally modify the activity of TLR-4 or alter the incorporation of TLR-4 to the plasma membrane, alter TLR-4 functionality (e.g., antibodies or small molecule drugs), alter TLR-4 trafficking and/or recycling, or alter TLR-4 signaling by pharmacological or gene therapy interventions upstream and/or downstream within the TLR-4 signaling pathway.

In some aspects, the present disclosure provides a nucleic acid aptamer for use in ameliorating or improving at least a symptom or sequelae of a disease or condition in a subject in need thereof, wherein

(a) the aptamer has a length, e.g., between about 40 and about 100 nucleotides and is selected from the group consisting of SEQ ID NOS: 1, 2, 3, and 4, wherein
(i) the aptamer specifically binds to an epitope on the extracellular domain of TLR-4; and,
(ii) binding of the aptamer to the epitope reduces and/or inhibits TLR-4 activation; or

(b) the aptamer is a functional equivalent variant of the aptamer of (a) having, e.g., at least 85% sequence identity to SEQ ID NO: 1, 2, 3, or 4, wherein the functionally equivalent variant is derived from SEQ ID NO: 1, 2, 3, or 4, and maintains the capability of specifically binding to and reducing and/or inhibiting TLR-4 activation.

In some aspects, the present disclosure provides a method to treat a disease or condition disclosed herein comprising the administration of a nucleic acid to a subject in need thereof, wherein

(a) the aptamer has a length, e.g., between about 40 and about 100 nucleotides and is selected from the group consisting of SEQ ID NOS: 1, 2, 3, and 4, wherein
(i) the aptamer specifically binds to an epitope on the extracellular domain of TLR-4; and,
(ii) binding of the aptamer to the epitope reduces and/or inhibits TLR-4 activation; or

(b) the aptamer is a functional equivalent variant of the aptamer of (a) having, e.g., at least 85% sequence identity to SEQ ID NO: 1, 2, 3, or 4, wherein the functionally equivalent variant is derived from SEQ ID NO: 1, 2, 3, or 4, and maintains the capability of specifically binding to and reducing and/or inhibiting TLR-4 activation.

In some aspects, the present disclosure provides a method for ameliorating, improving, inhibiting, or reducing at least a symptom or sequelae of a disease or condition disclosed herein in a subject in need thereof comprising the administration of a nucleic acid to the subject, wherein

(a) the aptamer has a length, e.g., between about 40 and about 100 nucleotides and is selected from the group consisting of SEQ ID NOS: 1, 2, 3, and 4, wherein
(i) the aptamer specifically binds to an epitope on the extracellular domain of TLR-4; and,
(ii) binding of the aptamer to the epitope reduces and/or inhibits TLR-4 activation; or

(b) the aptamer is a functional equivalent variant of the aptamer of (a) having, e.g., at least 85% sequence identity to SEQ ID NO: 1, 2, 3, or 4, wherein the functionally equivalent variant is derived from SEQ ID NO: 1, 2, 3, or 4, and maintains the capability of specifically binding to and reducing and/or inhibiting TLR-4 activation.

In some aspects, the methods disclosed herein can be practiced any of the aptamers disclosed in TABLE 1, or a combination thereof. Accordingly, in some aspects, the aptamer having a length, e.g., between about 40 and about 100 nucleotides, is selected from the group consisting of SEQ ID NOS: 1-16.

In some aspects, the aptamer having a length, e.g., between about 40 and about 100 nucleotides, is a functional equivalent variant having, e.g., at least 85% sequence identity to an aptamer of SEQ ID NO: 1-16, wherein the functionally equivalent variant is derived from SEQ ID NO: 1-16, and maintains the capability of specifically binding to and reducing and/or inhibiting TLR-4 activation.

In some aspects, the aptamer has a length of about 45, about 59, about 68, about 76, or about 78 nucleotides. In some aspects, the aptamer has a length between about 45 and about 78 nucleotides. In some aspects, the aptamer has a length between about 59 and about 78 nucleotides. In some aspects, the aptamer has a length between about 68 and about 78 nucleotides. In some aspects, the aptamer has a length between about 45 and about 76 nucleotides. In some aspects, the aptamer has a length between about 45 and about 68 nucleotides. In some aspects, the aptamer has a length between about 45 and about 59 nucleotides. In some aspects, the aptamer has a length between about 59 and about 76 nucleotides. In some aspects, the aptamer has a length between about 59 and about 68 nucleotides. In some aspects, the aptamer has a length between about 68 and about 76 nucleotides.

In some aspects, administration of an aptamer of the present disclosure (e.g., ApTOLL) or a combination thereof to a subject having an ischemic condition and/or thrombi can decrease the infarct volume. In some aspects, administration of an aptamer of the present disclosure (e.g., ApTOLL) or a combination thereof to a subject having an ischemic conditions and/or thrombi can decrease the infarct volume after administration of multiples dose of the aptamer of the present disclosure (e.g., ApTOLL) or a combination thereof, e.g., one, two, three, four, or five doses.

In some aspects, the administration of multiple doses of the aptamer of the present disclosure (e.g., ApTOLL) or a combination thereof, can start, e.g., about 5 minutes, about 10 minutes, about 15 minutes, about 20 minute, about 25 minutes, about 30 minutes, about 1 hour, about 2 hours, about 3 hours, about 4 hours, about 5 hours, about 6 hours, about 12 hours, about 18 hours, about 24 hours, about 30 hours, about 36 hours, about 42, or about 48 hours after occlusion. In some aspects, a single dose is administered, e.g., about 10 minutes after occlusion, wherein the administration of the aptamer induces a decrease in the infarct volume compared to control conditions, e.g., compared to infarct volumes in subjects not treated with the aptamer.

In some aspects, two doses are administered, e.g., about 10 minutes and about 2 hours after occlusion. In some aspects, three doses are administered, e.g., about 10 minutes, about 2 hours, and about 6 hours after occlusion. In some aspects, four doses are administered, e.g., about 10 minutes, about 2 hours, about 6 hours, and about 24 hours after occlusion. In some aspects, five doses are administered, e.g., about 10 min, about 2 hours, about 6 hours, about 24 hours, and about 48 hours after occlusion. In some aspects, such dose regimens induce a decrease in the infarct volume compared to control conditions, e.g., compared to infarct volumes in subjects not treated with the aptamer.

In some aspects, the administration of an aptamer of the present disclosure (e.g., ApTOLL) or a combination thereof induces an infarct volume reduction of at least about 10%, at least 15%, at least about 20%, or at least about 25%, compared to control conditions, e.g., compared to infarct volumes in subjects not treated with the aptamer.

In some aspects, administration of an aptamer of the present disclosure (e.g., ApTOLL) to a subject having an ischemic condition and/or thrombi reduces infarct volume when administered immediately after the ischemic event, e.g., a about 5 minutes, about 10 minutes, about 15 minutes, about 20 minutes, or about 30 minutes after the ischemic event. In some aspects, the reduction of infarct volume is about 20%, about 25%, about 30%, about 35%, about 40%, about 45%, about 50%, about 55%, about 60%, about 65%, about 70%, or about 75% compared to the infarcted volumes observed under control conditions, e.g., compared to infarct volumes in subjects not treated with an aptamer of the present disclosure (e.g., ApTOLL).

In some aspect, administration of an aptamer of the present disclosure (e.g., ApTOLL) intravenously to a subject having an ischemic condition and/or thrombi reduces infarct volume by about 65% when administered about 10 minutes after the ischemic event.

The present disclosure also provides a method to select a subject having an ischemic condition and/or thrombi for treatment with an aptamer of the present disclosure (e.g., ApTOLL), wherein the subject is selected from treatment if, e.g., blood vessel occlusion which is suitable for mechanical thrombectomy, e.g., determined or confirmed by Computerized Tomography Angiography (CTA). In some aspect, the criterion used for selection is large vessel occlusion, suitable for mechanical thrombectomy as determined or confirmed by neuroimaging criteria (CT or MRI), such as:

(i) Magnetic resonance imaging (MRI) criterion: volume of diffusion-weighted imaging (DWI) restriction about 5 mL and about 70 mL as determined, e.g., by RAPID® software; and/or,

(ii) Computerized tomography (CT) criterion: Alberta Stroke Program Early CT Score (ASPECTS) about 6 to about 10 and infarct score determined on admission cerebral blood flow (CBF)<30% and about 5 mL and about 70 mL determined, e.g., by RAPID® software.

In some aspect, the criterion used for the selection of the subject is the time from the onset of symptoms. Accordingly, in some aspects, the subject is selected for treatment with the aptamer of the present disclosure (e.g., ApTOLL) if less than 6 hours, e.g., less than 5 hours, less than 4 hours, less than 3 hours, less than 2 hours, or less than 1 hour, have elapsed since the onset of the ischemic condition and/or thrombi.

In some aspect, the criterion used for the selection of the subject for treatment with the aptamer of the present disclosure (e.g., ApTOLL) is whether the subject is a candidate to receive EVT treatment, e.g., a thrombectomy.

In some aspects, the subject is a human subject, and the aptamer of the present disclosure (e.g., ApTOLL) is administered at a dose between about 0.007 mg/kg and about 0.2 mg/kg. Accordingly, in some aspects, the aptamer of the present disclosure (e.g., ApTOLL) is administered to a human subject to treat any of the diseases or conditions disclosed herein, or to prevent, inhibit, or reduce any of the symptoms and/or sequelae associated with such disease or condition at a dosage of about 0.007 mg/kg per dose, about 0.008 mg/kg per dose, about 0.009 mg/kg per dose, about 0.010 mg/kg per dose, about 0.011 mg/kg per dose, about 0.012 mg/kg per dose, about 0.013 mg/kg per dose, about 0.014 mg/kg per dose, about 0.015 mg/kg per dose, about 0.016 mg/kg per dose, about 0.017 mg/kg per dose, about 0.018 mg/kg per dose, about 0.019 mg/kg per dose, about 0.020 mg/kg per dose, about 0.021 mg/kg per dose, about 0.022 mg/kg per dose, about 0.023 mg/kg per dose, about 0.024 mg/kg per dose, about 0.025 mg/kg per dose, about 0.030 mg/kg per dose, about 0.035 mg/kg per dose, about 0.040 mg/kg per dose, about 0.045 mg/kg per dose, about 0.050 mg/kg per dose, about 0.055 mg/kg per dose, about 0.060 mg/kg per dose, about 0.065 mg/kg per dose, about 0.070 mg/kg per dose, about 0.075 mg/kg per dose, about 0.080 mg/kg per dose, about 0.085 mg/kg per dose, about 0.090 mg/kg per dose, about 0.095 mg/kg per dose, about 0.100 mg/kg per dose, about 0.105 mg/kg per dose, about 0.110 mg/kg per dose, about 0.115 mg/kg per dose, about 0.120 mg/kg per dose, about 0.125 mg/kg per dose, about 0.130 mg/kg per dose, about 0.135 mg/kg per dose, about 0.140 mg/kg per dose, about 0.145 mg/kg per dose, about 0.150 mg/kg per dose, about 0.155 mg/kg per dose, about 0.160 mg/kg per dose, about 0.165 mg/kg per dose, about 0.170 mg/kg per dose, about 0.175 mg/kg per dose, about 0.180 mg/kg per dose, about 0.185 mg/kg per dose, about 0.190 mg/kg per dose, or about 0.2 mg/kg per dose.

The amount of a standard single dose, according to the disclosures above, considering a dose range between about 0.007 mg/kg and about 0.20 mg/kg, and considering a standard weight of the human subject of about 70 kg, is between about 0.5 mg/dose and about 10 mg/dose. Accordingly, in some aspects, the aptamer of the present disclosure (e.g., ApTOLL) is administered to a human subject to treat any of the diseases or conditions disclosed herein, or to prevent, inhibit, or reduce any of the symptoms and/or sequelae associated with such disease or condition at a dosage of about 0.5 mg/dose, about 0.6 mg/dose, about 0.7 mg/dose, about 0.8 mg/dose, about 0.9 mg/dose, about 1 mg/dose, about 1.1 mg/dose, about 1.2 mg/dose, about 1.3 mg/dose, about 1.4 mg/dose, about 1.5 mg/dose, about 1.6 mg/dose, about 1.7 mg/dose, about 1.8 mg/dose, about 1.9 mg/dose, about 2 mg/dose, about 2.5 mg/dose, about 3 mg/dose, about 3.5 mg/dose, about 4 mg/dose, about 4.5 mg/dose, about 5 mg/dose, about 5.5 mg/dose, about 6 mg/dose, about 6.5 mg/dose, about 7 mg/dose, about 7.5 mg/dose, about 8 mg/dose, about 8.5 mg/dose, about 9 mg/dose, about 9.5 mg/dose, about 10 mg/dose, about 11 mg/dose, about 12 mg/dose, about 13 mg/dose, about 14 mg/dose, about 15 mg/dose, about 16 mg/dose, about 17 mg/dose, about 18 mg/dose, about 19 mg/dose, or about 20 mg/dose.

In some aspects, the present disclosure provides a prophylactic method to prevent the development of an inflammatory response a subject having suffered acute myocardial infarction comprising the administration of an aptamer of the present disclosure (e.g., ApTOLL).

In some aspects, the present disclosure provides a method to selecting a subject having suffered acute myocardial infarction for treatment with an aptamer of the present disclosure (e.g., ApTOLL) comprising, e.g., (i) conducting a measurement, evaluation, or quantification of the infarcted area, (ii) assessing cardiac function, (iii) measurement of biomarkers related to tissue damage or tissue remodeling, or (iv) combinations thereof.

The present disclosure also provides a method to promote or induce recovery of heart function is a subject having suffered acute myocardial infarction, the method comprising administering an aptamer of the present disclosure (e.g., ApTOLL) to the subject. Also provided is a method to reduce necrosis (e.g., left ventricle necrosis) and/or fibrosis in a subject having suffered acute myocardial infarction, the method comprising administering an aptamer of the present disclosure (e.g., ApTOLL) to the subject. In some aspects, recovery of heart function, reduction in infarcted area with respect to controls, reduction of necrosis (e.g., left ventricle necrosis) with respect to controls, reduction of fibrosis with respect to controls, or any combination thereof can be observed at least 1 day, 2 days, 3 days, 4 days, 5 days, 6 days, 7 days, 8 days, 9 days, 10 days, 11 days, 12 days, 13 days, or 14 days after administering an aptamer of the present disclosure (e.g., ApTOLL) to the subject. In some aspects, troponin I levels in a subject having suffered acute myocardial infarction and who has been administered an aptamer of the present disclosure (e.g., ApTOLL) are lower than troponin I levels is subjects that have not been administered the aptamer. In some aspect, the lower troponin I levels are detectable, for example, about 2 hours, about 4 hours, about 6 hours, about 8 hours, about 10 hours, about 12 hours, about 14 hours, about 16 hours, about 18 hours, about 20 hours, about 22 hours, about 24 hours, about 30 hours, about 36 hours, about 42 hours, or about 48 hours after administering an aptamer of the present disclosure (e.g., ApTOLL) to the subject.

In some aspects, administration of an aptamer of the present disclosure (e.g., ApTOLL) to a subject having suffered acute myocardial infarction can reduce the infarct area by at least about 5%, at least about 10%, at least about 15%, at least about 20%, at least about 25%, at least about 30%, at least about 35%, at least about 40%, at least about 45%, or at least about 50% with respect to control conditions, e.g., with respect to subjects that have not been administered an aptamer of the present disclosure (e.g., ApTOLL).

In some aspects, administration of an aptamer of the present disclosure (e.g., ApTOLL) to a subject having suffered acute myocardial infarction can

(i) reduce the infarcted area (e.g., the volume of the infarcted area) with respect to controls;

(ii) preserve the integrity of cardiac tissue;

(iii) reduce or inhibit fibrosis;

(iv) inhibit the expression of markers for degradation of extracellular matrix;

(v) reduce, reduce the risk, or inhibit erroneous cardiac remodeling;

(vi) induce cardioprotection;

(vii) reduce or inhibit extracellular matrix degradation;

(viii) improve or promote cardiac remodeling;

(ix) preserve ventricular anatomy;

(x) preserve cardiac function;

(xi) reduce the progression of the infarction

(xii) improve myocardial repair

(xiii) increase or recovery in ventricle contractility; or,

(xiv) any combination thereof.

Accordingly, in some aspects, the present disclosure provides methods to

(i) reduce the infarcted area (e.g., the volume of the infarcted area) with respect to controls;

(ii) preserve the integrity of cardiac tissue;

(iii) reduce or inhibit fibrosis;

(iv) inhibit the expression of markers for degradation of extracellular matrix;

(v) reduce, reduce the risk, or inhibit erroneous cardiac remodeling;

(vi) induce cardioprotection;

(vii) reduce or inhibit extracellular matrix degradation;

(viii) improve or promote cardiac remodeling;

(ix) preserve ventricular anatomy;

(x) preserve cardiac function;

(xi) reduce the progression of the infarction;

(xii) improve myocardial repair;

(xiii) increase or recovery in ventricle contractility; or,

(xiv) any combination thereof;

in a subject having suffered acute myocardial infarction comprising administering an aptamer of the present disclosure (e.g., ApTOLL) to the subject.

In some aspects, the expression of MMP-9 in a subject having suffered acute myocardial infarction comprising having being administering an aptamer of the present disclosure (e.g., ApTOLL) to the subject is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%, with respect to the expression in a subject that has not been administered an aptamer of the present disclosure (e.g., ApTOLL).

In some aspects, the present disclosure provides methods to select a subject having suffered acute myocardial infarction for administration of an aptamer of the present disclosure (e.g., ApTOLL), the method comprising measuring the expression levels of MMP-9 in the subject (e.g., protein expression levels, mRNA expression levels, or a combination thereof), and administering an aptamer of the present disclosure (e.g., ApTOLL) if the MMP-9 is elevated with respect to control values, e.g., values observer in subjects not treated with an aptamer of the present disclosure (e.g., ApTOLL) or standard normal expression values.

In some aspects, the aptamer of the present disclosure (e.g., ApTOLL) is administered to a human subject having suffered acute myocardial infarction at a dose between about 0.007 mg/kg and about 0.20 mg/kg. Accordingly, in some aspects, the aptamer of the present disclosure (e.g., ApTOLL) is administered to a human subject having suffered acute myocardial infarction at a dosage of about 0.007 mg/kg per dose, about 0.008 mg/kg per dose, about 0.009 mg/kg per dose, about 0.010 mg/kg per dose, about 0.011 mg/kg per dose, about 0.012 mg/kg per dose, about 0.013 mg/kg per dose, about 0.014 mg/kg per dose, about 0.015 mg/kg per dose, about 0.016 mg/kg per dose, about 0.017 mg/kg per dose, about 0.018 mg/kg per dose, about 0.019 mg/kg per dose, about 0.020 mg/kg per dose, about 0.021 mg/kg per dose, about 0.022 mg/kg per dose, about 0.023 mg/kg per dose, about 0.024 mg/kg per dose, about 0.025 mg/kg per dose, about 0.030 mg/kg per dose, about 0.035 mg/kg per dose, about 0.040 mg/kg per dose, about 0.045 mg/kg per dose, about 0.050 mg/kg per dose, about 0.055 mg/kg per dose, about 0.060 mg/kg per dose, about 0.065 mg/kg per dose, about 0.070 mg/kg per dose, about 0.075 mg/kg per dose, about 0.080 mg/kg per dose, about 0.085 mg/kg per dose, about 0.090 mg/kg per dose, about 0.095 mg/kg per dose, about 0.100 mg/kg per dose, about 0.105 mg/kg per dose, about 0.110 mg/kg per dose, about 0.115 mg/kg per dose, about 0.120 mg/kg per dose, about 0.125 mg/kg per dose, about 0.130 mg/kg per dose, about 0.135 mg/kg per dose, about 0.140 mg/kg per dose, about 0.145 mg/kg per dose, about 0.150 mg/kg per dose, about 0.155 mg/kg per dose, about 0.160 mg/kg per dose, about 0.165 mg/kg per dose, about 0.170 mg/kg per dose, about 0.175 mg/kg per dose, about 0.180 mg/kg per dose, about 0.185 mg/kg per dose, about 0.190 mg/kg per dose, or about 0.2 mg/kg per dose.

The amount of a standard single dose, according to the disclosures above, considering a dose range between about 0.007 mg/kg and about 0.20 mg/kg, and considering a standard weight of the human subject having suffered acute myocardial infarction of about 70 kg, is between about 0.5 mg/dose and about 10 mg/dose. Accordingly, in some aspects, the aptamer of the present disclosure (e.g., ApTOLL) is administered to a human subject having suffered acute myocardial infarction at a dosage of about 0.5 mg/dose, about 0.6 mg/dose, about 0.7 mg/dose, about 0.8 mg/dose, about 0.9 mg/dose, about 1 mg/dose, about 1.1 mg/dose, about 1.2 mg/dose, about 1.3 mg/dose, about 1.4 mg/dose, about 1.5 mg/dose, about 1.6 mg/dose, about 1.7 mg/dose, about 1.8 mg/dose, about 1.9 mg/dose, about 2 mg/dose, about 2.5 mg/dose, about 3 mg/dose, about 3.5 mg/dose, about 4 mg/dose, about 4.5 mg/dose, about 5 mg/dose, about 5.5 mg/dose, about 6 mg/dose, about 6.5 mg/dose, about 7 mg/dose, about 7.5 mg/dose, about 8 mg/dose, about 8.5 mg/dose, about 9 mg/dose, about 9.5 mg/dose, about 10 mg/dose, about 11 mg/dose, about 12 mg/dose, about 13 mg/dose, about 14 mg/dose, about 15 mg/dose, about 16 mg/dose, about 17 mg/dose, about 18 mg/dose, about 19 mg/domes or about 20 mg/dose.

In some aspects, the present disclosure provides prevent, inhibit, suppress, or delay, the onset of a symptom and/or sequelae of a neuromuscular o neurodegenerative disease or condition, e.g., multiple sclerosis, in a subject in need thereof comprising administering to the subject an aptamer of the present disclosure (e.g., ApTOLL). In some aspects, the aptamer of the present disclosure (e.g., ApTOLL) is administering to the subject about 24 hours after the onset of the symptoms of the neuromuscular o neurodegenerative disease or condition. In some aspects, a single dose of aptamer of the present disclosure (e.g., ApTOLL) is administered to the subject. In some aspects, more than one dose of aptamer of the present disclosure (e.g., ApTOLL) is administering to the subject, e.g., two, three, four, or five doses.

In some aspects, administering an aptamer of the present disclosure (e.g., ApTOLL) to a subject having a neuromuscular o neurodegenerative disease or condition results in (i) improvement in clinical scores, (ii) reduced weight loss (weight recovery), (iii) remyelination, (iv) reduction in axonal damage, (v) reduction in inflammation, (vi) reduction in demyelination, (vii) increase in myelin area, (viii) increase in neurofilaments, or (ix) any combination. Accordingly, in some aspects, the present disclosure provides a method to (i) improve clinical scores, (ii) reduced weigh loss (recover weight), (iii) remyelinize, (iv) reduce axonal damage, (v) reduce inflammation, (vi) reduce demyelination, (vii) increase myelin area, (viii) increase neurofilaments, or (ix) any combination, in a subject having a neuromuscular o neurodegenerative disease or condition, the method comprising administering an aptamer of the present disclosure (e.g., ApTOLL) to the subject. In some aspects, remyelinization can be determine by measuring levels of biomarkers such as PDGFRα, CC1, Oligo2, or a combination thereof.

In some aspects, the aptamer of the present disclosure (e.g., ApTOLL) is administered to a human subject having a neuromuscular or neurodegenerative disease or condition at a dose between about 0.007 mg/kg and about 0.20 mg/kg. Accordingly, in some aspects, the aptamer of the present disclosure (e.g., ApTOLL) is administered to a human subject having a neuromuscular or neurodegenerative disease or condition at a dosage of about 0.007 mg/kg per dose, about 0.008 mg/kg per dose, about 0.009 mg/kg per dose, about 0.010 mg/kg per dose, about 0.011 mg/kg per dose, about 0.012 mg/kg per dose, about 0.013 mg/kg per dose, about 0.014 mg/kg per dose, about 0.015 mg/kg per dose, about 0.016 mg/kg per dose, about 0.017 mg/kg per dose, about 0.018 mg/kg per dose, about 0.019 mg/kg per dose, about 0.020 mg/kg per dose, about 0.021 mg/kg per dose, about 0.022 mg/kg per dose, about 0.023 mg/kg per dose, about 0.024 mg/kg per dose, about 0.025 mg/kg per dose, about 0.030 mg/kg per dose, about 0.035 mg/kg per dose, about 0.040 mg/kg per dose, about 0.045 mg/kg per dose, about 0.050 mg/kg per dose, about 0.055 mg/kg per dose, about 0.060 mg/kg per dose, about 0.065 mg/kg per dose, about 0.070 mg/kg per dose, about 0.075 mg/kg per dose, about 0.080 mg/kg per dose, about 0.085 mg/kg per dose, about 0.090 mg/kg per dose, about 0.095 mg/kg per dose, about 0.100 mg/kg per dose, about 0.105 mg/kg per dose, about 0.110 mg/kg per dose, about 0.115 mg/kg per dose, about 0.120 mg/kg per dose, about 0.125 mg/kg per dose, about 0.130 mg/kg per dose, about 0.135 mg/kg per dose, about 0.140 mg/kg per dose, about 0.145 mg/kg per dose, about 0.150 mg/kg per dose, about 0.155 mg/kg per dose, about 0.160 mg/kg per dose, about 0.165 mg/kg per dose, about 0.170 mg/kg per dose, about 0.175 mg/kg per dose, about 0.180 mg/kg per dose, about 0.185 mg/kg per dose, about 0.190 mg/kg per dose, or about 0.2 mg/kg per dose.

The amount of a standard single dose, according to the disclosures above, considering a dose range between about 0.007 mg/kg and about 0.20 mg/kg, and considering a standard weight of the human subject having a neuromuscular or neurodegenerative disease or condition of about 70 kg, is between about 0.5 mg/dose and about 10 mg/dose. Accordingly, in some aspects, the aptamer of the present disclosure (e.g., ApTOLL) is administered to a human subject having a neuromuscular or neurodegenerative disease or condition at a dosage of about 0.5 mg/dose, about 0.6 mg/dose, about 0.7 mg/dose, about 0.8 mg/dose, about 0.9 mg/dose, about 1 mg/dose, about 1.1 mg/dose, about 1.2 mg/dose, about 1.3 mg/dose, about 1.4 mg/dose, about 1.5 mg/dose, about 1.6 mg/dose, about 1.7 mg/dose, about 1.8 mg/dose, about 1.9 mg/dose, about 2 mg/dose, about 2.5 mg/dose, about 3 mg/dose, about 3.5 mg/dose, about 4 mg/dose, about 4.5 mg/dose, about 5 mg/dose, about 5.5 mg/dose, about 6 mg/dose, about 6.5 mg/dose, about 7 mg/dose, about 7.5 mg/dose, about 8 mg/dose, about 8.5 mg/dose, about 9 mg/dose, about 9.5 mg/dose, about 10 mg/dose, about 11 mg/dose, about 12 mg/dose, about 13 mg/dose, about 15 mg/dose, about 16 mg/dose, about 17 mg/dose, about 18 mg/dose, about 19 mg/dose, or about 20 mg/dose.

Also provided in a method to prevent, inhibit, suppress, or delay, the onset of a symptom and/or sequelae in a pathology comprising primary and secondary demyelization such as stroke or cranioencephalic trauma (traumatic brain injury) in a subject comprising administering an aptamer of the present disclosure (e.g., ApTOLL) to the subject.

In some aspects, the aptamer of the present disclosure (e.g., ApTOLL) is administered to a human subject having suffered stroke or a traumatic brain injury at a dose between about 0.007 mg/kg and about 0.20 mg/kg. Accordingly, in some aspects, the aptamer of the present disclosure (e.g., ApTOLL) is administered to a human subject having suffered stroke or a traumatic brain injury at a dosage of about 0.007 mg/kg per dose, about 0.008 mg/kg per dose, about 0.009 mg/kg per dose, about 0.010 mg/kg per dose, about 0.011 mg/kg per dose, about 0.012 mg/kg per dose, about 0.013 mg/kg per dose, about 0.014 mg/kg per dose, about 0.015 mg/kg per dose, about 0.016 mg/kg per dose, about 0.017 mg/kg per dose, about 0.018 mg/kg per dose, about 0.019 mg/kg per dose, about 0.020 mg/kg per dose, about 0.021 mg/kg per dose, about 0.022 mg/kg per dose, about 0.023 mg/kg per dose, about 0.024 mg/kg per dose, about 0.025 mg/kg per dose, about 0.030 mg/kg per dose, about 0.035 mg/kg per dose, about 0.040 mg/kg per dose, about 0.045 mg/kg per dose, about 0.050 mg/kg per dose, about 0.055 mg/kg per dose, about 0.060 mg/kg per dose, about 0.065 mg/kg per dose, about 0.070 mg/kg per dose, about 0.075 mg/kg per dose, about 0.080 mg/kg per dose, about 0.085 mg/kg per dose, about 0.090 mg/kg per dose, about 0.095 mg/kg per dose, about 0.100 mg/kg per dose, about 0.105 mg/kg per dose, about 0.110 mg/kg per dose, about 0.115 mg/kg per dose, about 0.120 mg/kg per dose, about 0.125 mg/kg per dose, about 0.130 mg/kg per dose, about 0.135 mg/kg per dose, about 0.140 mg/kg per dose, about 0.145 mg/kg per dose, about 0.150 mg/kg per dose, about 0.155 mg/kg per dose, about 0.160 mg/kg per dose, about 0.165 mg/kg per dose, about 0.170 mg/kg per dose, about 0.175 mg/kg per dose, about 0.180 mg/kg per dose, about 0.185 mg/kg per dose, about 0.190 mg/kg per dose, or about 0.2 mg/kg per dose.

The amount of a standard single dose, according to the disclosures above, considering a dose range between about 0.007 mg/kg and about 0.20 mg/kg, and considering a standard weight of a human subject having suffered stroke or a traumatic brain injury of about 70 kg, is between about 0.5 mg/dose and about 10 mg/dose. Accordingly, in some aspects, the aptamer of the present disclosure (e.g., ApTOLL) is administered to a human subject having suffered stroke or a traumatic brain injury at a dosage of about 0.5 mg/dose, about 0.6 mg/dose, about 0.7 mg/dose, about 0.8 mg/dose, about 0.9 mg/dose, about 1 mg/dose, about 1.1 mg/dose, about 1.2 mg/dose, about 1.3 mg/dose, about 1.4 mg/dose, about 1.5 mg/dose, about 1.6 mg/dose, about 1.7 mg/dose, about 1.8 mg/dose, about 1.9 mg/dose, about 2 mg/dose, about 2.5 mg/dose, about 3 mg/dose, about 3.5 mg/dose, about 4 mg/dose, about 4.5 mg/dose, about 5 mg/dose, about 5.5 mg/dose, about 6 mg/dose, about 6.5 mg/dose, about 7 mg/dose, about 7.5 mg/dose, about 8 mg/dose, about 8.5 mg/dose, about 9 mg/dose, about 9.5 mg/dose, about 10 mg/dose, about 11 mg/dose, about 12 mg/dose, about 13 mg/dose, about 14 mg/dose, about 15 mg/dose, about 16 mg/dose, about 17 mg/dose, about 18 mg/dose, about 19 mg/dose, or about 20 mg/dose.

### III. APTAMERS SPECIFIC FOR TLR-4

The aptamers used in the methods of the present disclosure have the capability of binding specifically to at least one epitope located on the extracellular domain of TLR-4, and inhibiting TLR-4. Specific examples of aptamers of the present disclosure are presented in TABLE 1. In some aspects, the aptamer of the present disclosure is a variant and/or a derivative of an aptamer disclosed in TABLE 1.

TABLE 1
Exemplary aptamers specific for TLR-4

| SEQ | | |
|---|---|---|
| ID | | |
| NO | Sequence | Length |
| 1 | CCGGCACGGGACAAGGCGCGGGACGGCGTAGATCAGGTCGACACC | 45 |
| 2 | GGTGTGCCAATAAACCATATCGCCGCGTTAGCATGTACTCGGTTGGCCCTAAATACGAG | 59 |
| 3 | GTTGCTCGTATTTAGGGCCACCGGCACGGGACAAGGCGCGGGACGGCGTAGATCAGGTCGAC | 78 |
| | ACCAGTCTTCATCCGC | |
| 4 | GCGGATGAAGACTGGTGTGCCAATAAACCATATCGCCGCGTTAGCATGTACTCGGTTGGCCC | 76 |
| | TAAATACGAGCAAC | |
| 5 | GTTGCTCGTATTTAGGGCCACCGGCACGGGACAAGGCGCGGGACGGCGTAGATCAGGTCGAC | 78 |
| | ACCAGTCTTCATCCGC | |
| 6 | GCGGATGAAGACTGGTGTCGACCTGATCTACGCCGTCCCGCGCCTTGTCCCGTGCCGGTGGC | 78 |
| | CCTAAATACGAGCAAC | |
| 7 | GTTGCTCGTATTTAGGGCACACACGCACGAAGACCTTGGCTGCCCGTTGTACACCAGTCTTC | 68 |
| | ATCCGC | |
| 8 | GCGGATGAAGACTGGTGTACAACGGGCAGCCAAGGTCTTCGTGCGTGTGTGCCCTAAATACG | 68 |
| | AGCAAC | |
| 9 | GTTGCTCGTATTTAGGGCACCGAGGTCACCGAACTTGGTGTGCACAGTTGTTGGCGCGACAC | 76 |
| | CAGTCTTCATCCGC | |
| 10 | GCGGATGAAGACTGGTGTCGCGCCAACAACTGTGCACACCAAGTTCGGTGACCTCGGTGCCC | 76 |
| | TAAATACGAGCAAC | |
| 11 | GTTGCTCGTATTTAGGGCCACATATGTGCACATCACAATCCGCAGAGCTGCACCTACGACAC | 76 |
| | CAGTCTTCATCCGC | |
| 12 | GCGGATGAAGACTGGTGTCGTAGGTGCAGCTCTGCGGATTGTGATGTGCACATATGTGGCCC | 76 |
| | TAAATACGAGCAAC | |
| 13 | GTTGCTCGTATTTAGGGCCAAGGAAAACCCCCTGGTCACTGGTACTAATCCGATCCGTACAC | 76 |
| | CAGTCTTCATCCGC | |
| 14 | GCGGATGAAGACTGGTGTACGGATCGGATTAGTACCAGTGACCAGGGGGTTTTCCTTGGCCC | 76 |
| | TAAATACGAGCAAC | |
| 15 | GTTGCTCGTATTTAGGGCGGGTCACCACGGAAGAGTGTAGATACATAGATACAGTCCGACAC | 76 |
| | CAGTCTTCATCCGC | |
| 16 | GCGGATGAAGACTGGTGTCGGACTGTATCTATGTATCTACACTCTTCCGTGGTGACCCGCCC | 76 |
| | TAAATACGAGCAAC | |

The aptamers of TABLE 1 have lengths between 45 nucleotides to 78 nucleotides. The A content ranges from about 17% to about 27%. The T content ranges from about 17% to about 28%. The G content ranges from about 21% to about 33%. The C content ranges from about 20% to about 34%.

In some aspects, the aptamer of the present disclosure is a chemically modified aptamer as disclosed below. In some aspects, the aptamer of the present disclosure is a DNA and/or RNA aptamer (e.g., a ssDNA aptamer) that can bind specifically to and inhibit TLR-4 with at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% of the capability of specifically binding to and inhibiting TLR-4 of an aptamer of disclosed in TABLE 1.

In some aspects, the aptamer of the present disclosure comprises at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 120, 140, 160, 180, 200, 250, 300, 350, 400, 450, 500, 550, 600, 650, 700, 750, 800, 850, 900, 950, 1000, or more nucleotides at the 5′ of a sequence disclosed in TABLE 1, wherein the aptamer is capable of specifically binding to and inhibiting TLR-4.

In some aspects, the aptamer of the present disclosure comprises at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 120, 140, 160, 180, 200, 250, 300, 350, 400, 450, 500, 550, 600, 650, 700, 750, 800, 850, 900, 950, 1000, or more nucleotides at the 3′ of a sequence disclosed in TABLE 1, wherein the aptamer is capable of specifically binding to and inhibiting TLR-4.

In some aspects, the aptamer of the present disclosure comprises a nucleic acid sequence with at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% sequence identity to a sequence disclosed in TABLE 1, wherein the aptamer is capable of specifically binding to and inhibiting TLR-4.

In some aspects, the aptamer of the present disclosure consists of a nucleic acid sequence (e.g., a ssDNA) between about 30 and about 200 nucleotides, between about 35 and about 150 nucleotides, between about 40 and about 100 nucleotides, between about 45 and about 80 nucleotides, between about 40 and about 50 nucleotides, between about 35 and about 55 nucleotides, between 30 and about 60 nucleotides, between about 35 and about 65 nucleotides, between about 40 and about 70 nucleotides, between about 75 and about 85 nucleotides, between about 70 and about 90 nucleotides, between about 65 and about 95 nucleotides, between about 60 and about 100 nucleotides, between about 55 and about 95 nucleotides, between about 50 and about 90 nucleotides, between about 45 and about 85 nucleotides, between about 50 and about 80 nucleotides, between about 55 and about 75, or between about 60 and about 75 nucleotides.

In some aspects, an aptamer of the present disclosure can be covalently or non-covalently attached to at least one biologically active molecule. In some aspects, the biologically active molecule can specifically bind to TLR-4. In some aspects, the biologically active molecule comprises, e.g., an antibody or an antigen-binding fragment thereof, a small molecule, peptide, aptamer, lipid, lipopolysaccharide, polysaccharide, enzyme, or nucleic acid. In some aspects, the biologically active molecule comprises an anti-inflammatory.

In some aspects, the biologically active molecule is a TLR-4 antagonist selected from the group consisting of naloxone, (+)-naloxone, naltrexone, (+)-naltrexone, lipopolysaccharide (LPS), ibudilast, propentofylline, amitriptyline, ketotifen, cyclobenzaprine, mianserin, imipramine, a lipid A analog (e.g., eritoran or E5531), pinocembrin, palmitoylethanolamide, tapentadol, polypropyletherimine dendrimer glucosamine (DG), aminoalkyl glucosaminide 4-phosphate (e.g., CRX-526), IAXO-102, Rs-LPS, TLR-IN-C34, TAK-242, E5564, or any combination thereof.

In some aspects, the biologically active molecule comprises an anti-platelet drug, e.g., aspirin or clopidogrel. In some aspects, the biologically active molecule comprises an anti-coagulant, e.g., heparin, acenocumarol, warfarin, dabigatran, or rivaroxaban. In some aspects, the biologically active molecule comprises an antioxidant, e.g., edaravone. In some aspects, the biologically active molecule is tissue plasminogen activator.

In some aspects, the biologically active molecule is a beta blocker, e.g., metoprolol or cavedilol, an ACE inhibitor, a statin, or an aldosterone antagonist, e.g., spironolactone or eplerenone.

In some aspects, the biologically active molecule comprises a nucleic acid (e.g., antisense RNA, antisense DNA and small interfering RNA), which has the capability of silencing the expression of genes involved in a pathology characterized by an increase in expression of TLR-4 and/or an increase in activation of TLR-4, including, without limitation, the NFKB1, RIPK3, IFNB1, LY96 (MD-2), IRF3, TLR3, TIRAP (MaI), TICAM1 (TRIF), RIPK1, TRAF6, CD14, TRAM, IKBKG (IKK-gamma), IFNA1 and TLR4 genes. The term “antisense RNA,” in the context of the present disclosure, refers to a single-stranded RNA the nucleotide sequence of which is complementary for a target messenger RNA, thereby interfering with the expression of the respective gene. The term “antisense DNA,” in the context of the present disclosure, refers to a single-stranded DNA the nucleotide sequence of which is complementary for a target messenger RNA, thereby interfering with or silencing the expression of the respective gene. The term “small interfering RNA” or “siRNA,” in the context of the present disclosure, refers to a double-stranded RNA with a length of 20 to 25 nucleotides which is highly specific for the nucleotide sequence of its target messenger RNA, thereby interfering with the expression of the respective gene.

In some aspects, the aptamers of the present disclosure (e.g., ApTOLL) are resistant to degradation by X-exonuclease. In some aspects, the aptamers of the present disclosure (e.g., ApTOLL) are resistant to degradation by X-exonuclease, e.g., after incubation with the nuclease for at least about 5 minutes, at least about 15 minutes, at least about 30 minutes, at least about 1 hour, at least 2 hours or at least about 4 hours.

In some aspects, the aptamers of the present disclosure (e.g., ApTOLL) inhibit or reduce TLR-4 activation mediated by LPS (lipopolysaccharide), e.g., as measured using HEK-blue-hTLR-4 cells expressing hTLR-4 and the TLR-4 co-activator proteins MD2 and CD14 using methods known in the art. In some aspects, such reduction in activation is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the action observed under control conditions, e.g., without administration of an aptamer of the present disclosure (e.g., ApTOLL).

In some aspects, the aptamers of the present disclosure (e.g., ApTOLL) have a binding affinity for human TLR-4 of 30-60 nM, as measured using methods known in the art and cynomolgus monkey and human monocytes. In some aspects, the aptamers of the present disclosure have a binding affinity for human TLR-4 of at least about 20 nM, at least about 25 nM, at least about 30 nM, at least about 35 nM, at least about 40 nM, at least about 45 nM, at least about 50 nM, at least about 55 nM, at least about 60 nM, at least about 65 nM, or at least about 70 nM.

In some aspects, the aptamers of the present disclosure (e.g., ApTOLL) inhibit TLR-4 activation induced by damage associated molecular patterns (DAMPs), e.g., as measured using HEK-blue-hTLR-4 cells expressing hTLR-4 and the TLR-4 co-activator proteins MD2 and CD14 using methods known in the art. DAMPs (Damage-Associated Molecular Patterns) are tissue molecules such as heat-shock proteins, nucleic acids, fibronectin or hyaluronan, that are released in the brain parenchyma under damaging conditions. Thus, in some aspects, the aptamer of the present disclosure can inhibit TLR-4 activation by endogenous TLR-4 agonists (e.g., DAMPs) by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% compared to control conditions (e.g., without the administration of the aptamer of the present disclosure).

In some aspects, the aptamers of the present disclosure induce a reduction in downstream TLR-4 cell effectors, such as NOx levels, e.g., in murine peritoneal macrophages stimulated by LPS as measured using methods known in the art. In some aspects, the administration of aptamers of the present disclosure induces a reduction in NOx levels by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% compared to control conditions (e.g., without the administration of the aptamer of the present disclosure).

In some aspects, aptamers of the present disclosure have no detectable agonistic effect against TLR2, TLR3, TLR4, TLR5, TLR7, TLR8 or TLR9 human Toll receptors, and no antagonistic effect against TLR2 and TLR5.

In some aspects, TLR-4 receptors are internalized after binding to aptamers of the present disclosure, e.g., as measured in human macrophages using methods known in the art. In some aspects, TLR-4 receptors comprising bound aptamers of the present disclosure are internalized into the cytoplasm approximately 20 minutes after binding of the aptamer to TLR-4.

In some aspects, new TLR-4 receptors able to bind aptamers of the present disclosure are detected on the cell surface after TLR-4 internalization following binding of aptamers of the present disclosure to TLR-4 (i.e., internalized TLR-4 is recycled to the plasmatic membrane), e.g., as measured in human macrophages using methods known in the art.

In some aspects, the new TLR-4 receptors able to bind aptamers of the present disclosure are detected on the cell surface approximately 5 hours after TLR-4 internalization following binding of aptamers of the present disclosure to TLR-4.

In some aspects, administration of aptamers of the present disclosure to iPSC-derived cortical glutaminergic (80%) and GABAergic (20%) neurons results in no detectable toxicity to the neurons.

In some aspects, administration of an aptamer of the present disclosure to a subject in need thereof results in a decrease in proinflammatory cytokines. In some aspects, the proinflammatory cytokines are selected from the group consisting of interleukin-6 (IL-6), interferon-γ (IFN-γ), tumor necrosis factor alpha (TNF-α), interleukin-12p70 (IL-12p70), and any combination thereof.

In one aspect, administration of an aptamer of the present disclosure can result in a reduction in interferon-γ (IFN-γ) levels of at least about 5%, at least about 10%, at least 15%, at least about 20%, or at least about 25% compared to control conditions (e.g., without the administration of the aptamer of the present disclosure).

In one aspect, administration of an aptamer of the present disclosure can result in a reduction in interleukin-12p70 (IL-12p70) levels of at least about 5%, at least about 10%, at least 15%, at least about 20%, at least about 25%, at least about 30%, or at least about 35% compared to control conditions (e.g., without the administration of the aptamer of the present disclosure).

In one aspect, administration of an aptamer of the present disclosure can result in a reduction in tumor necrosis factor alpha (TNF-α) levels of at least about 5%, at least about 10%, or at least about 15% compared to control conditions (e.g., without the administration of the aptamer of the present disclosure).

In one aspect, administration of an aptamer of the present disclosure can result in a reduction in interleukin-6 (IL-6) levels of at least about 5%, at least about 10%, at least about 15%, at least about 20%, at least about 25%, at least about 30%, at least about 35%, at least about 40%, at least 45%, or at least about 50% compared to control conditions (e.g., without the administration of the aptamer of the present disclosure).

In some aspects, the aptamers of the present disclosure can be transported across the blood-brain barrier (BBB). In some aspects, the aptamers of the present disclosure can be transported across the BBB after the BBB has been compromised, e.g., by a hemorrhagic or ischemic event. Thus, in some aspects, the aptamers of the present disclosure cannot cross the BBB in healthy subjects.

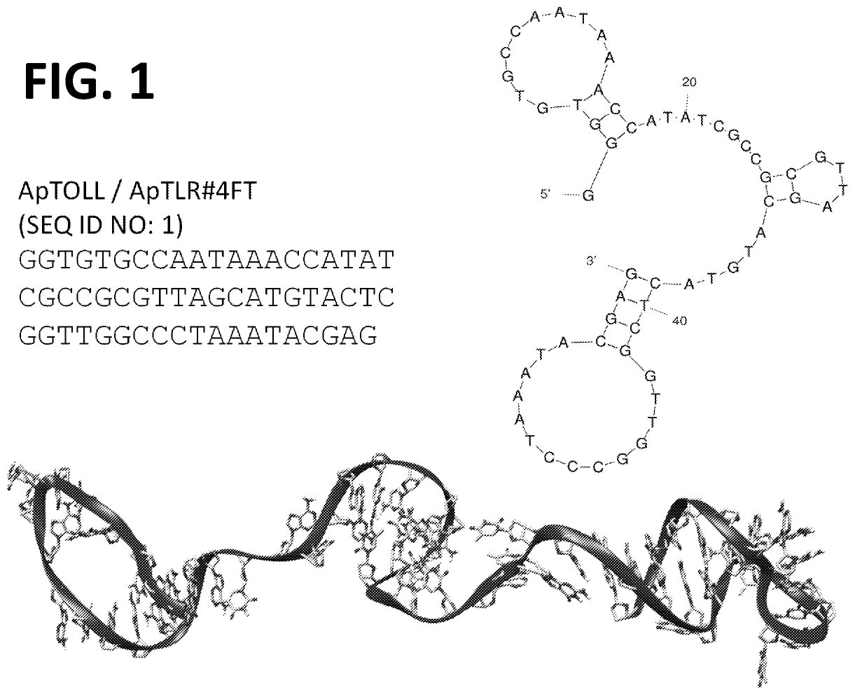

In one specific aspect, the aptamer of the present disclosure is ApTOLL. As used herein the term “ApTOLL” refers to a nucleic acid (single stranded DNA, ssDNA) aptamer that specifically binds to TLR-4 comprising the sequence of SEQ ID NO: 1. In a particular aspect, the term ApTOLL refers to a structured nucleic acid aptamer of SEQ ID NO: 1. As used herein, the terms “structured nucleic acid aptamer” or “structured aptamer” refers to a nucleic acid aptamer that has been linearized by exposure to denaturing conditions (e.g., high temperature, such as 95° C., for example for 10 minutes) and subsequently refolded at low temperature (e.g., by immersion in ice, for example, for 10 minutes) so it acquires a tertiary structure that allows the interaction between the structured aptamer, e.g., ApTOLL, and its target, e.g., an epitope on the extracellular domain of TLR-4. See FIG. 1.

The chemical formula of ApTOLL is C₅₇₅H₇₂₃N₂₂₃O₃₅₁P₅₈ and its molecular weight is 18,170.80 Da. The molecular sequence of ApTOLL has been confirmed through controlled enzymatic digest followed by MS-MS (Mass Spectrophotometry) sequencing. The correct structure has been assessed by confirming the expected biological activity in an in vitro assay. To adopt its biologically active conformation, the aptamer is dissolved in PBS-1 mM MgCl₂ and, after dissolution, the aptamer must be heated to 95° C. for about 10 minutes and then snap-cooled on ice for about 10 min. This buffer solution and conditions support the aptamer structure and its biological activity.

The dosage form of the investigational medicinal product (IMP) ApTOLL corresponds to a powder for concentrate for solution for infusion which consists of a freeze-dried powder to be reconstituted with water for injection and further diluted with saline solution for its intravenous administration.

ApTOLL has demonstrated specific binding to human TLR-4 as well as a TLR4 antagonistic effect. ApTOLL has shown, e.g., a long-lasting protective effect against brain injury induced by middle artery occlusion (MCAO). Additionally, efficacy of ApTOLL in models of brain ischemia-reperfusion support the use of this aptamer in patients undergoing artery recanalization induced by pharmacological and/or mechanical interventions.

Preclinical pharmacokinetic studies have demonstrated that Cₘₐₓ values of ApTOLL in rats appeared to be characterized by dose-independent (linear) kinetics over the dose range 0.45 to 2 mg/kg and the extent of systemic exposure of female rats to ApTOLL appeared to be characterized by nonlinear (dose-dependent) kinetics over the dose range 0.45 to 2 mg/kg. Increasing the dose of ApTOLL above 0.45 mg/kg is likely to result in a lower systemic exposure than would be predicted from a linear relationship, which is consistent with the possibility of an increase in plasma clearance of ApTOLL at higher dose levels. Pharmacodynamic, safety pharmacology, pharmacokinetic and toxicology nonclinical studies have been performed to characterize ApTOLL in three species: mice (C57Bl6, ICR), rats (Wistar and Sprague Dawley (SD)) and NHP (Non-Human Primates; Cynomolgus monkeys). These species were selected due to the receptor human-homology and TLR4 pharmacology.

Pharmacodynamic characterization performed in vitro and in vivo indicate that binding of ApTOLL to TLR-4 from human and non-human primates (NHP) has a Ka of approximately 30 to 60 nM, and also shows absence of binding of ApTOLL to other TLRs.

Pharmacodynamic characterization of the aptamers of the present disclosure, e.g., ApTOLL, in vivo indicates that, e.g., up to a 65.5% reduction of infarct volume can be observed after administration of the aptamer to a subject that has suffered an acute ischemic stroke. A therapeutic window of up a 12 hours has been observed. Administration of multiple doses of an aptamer of the present disclosure, e.g., ApTOLL, generally provides better protection than single dose administration. Administration of aptamers of the present disclosure, e.g., ApTOLL, to a subject suffering from acute ischemic stroke results in improved neurological outcome, both short term and long term. Experimental observation have confirmed that administration of aptamers of the present disclosure, e.g., ApTOLL, to a subject in need thereof results in a blockage of the inflammatory cascade. Furthermore, administration of aptamers of the present disclosure, e.g., ApTOLL, has not shown any drug-drug interaction with i.v. rt-PA.

Biodistribution studies shown that ApTOLL is mainly present in kidney, spleen and liver 1 hour after intravenous injection, both in naïve and ischemic subjects. 24 hours after injection, ApTOLL levels are almost undetectable. Under physiological conditions, ApTOLL is not able to cross the BBB in healthy subjects. However, ApTOLL is able to cross the BBB in individual that have experienced an ischemic event. When administered after an ischemic event, ApTOLL is mainly present in the ipsilateral hemisphere (i.e., the hemisphere that has suffered the ischemic event) of the brain of the subject.

Metabolism and distribution of ApTOLL have been determined both in vitro and in vivo. ApTOLL is degraded by exonucleases in plasma few minutes after administration. Neither drug-interactions nor inhibition of transporters or cytochrome were detected. In vivo regulatory pharmacokinetic studies performed in SD rats indicates that Tₘₐₓ was achieved 1 min post-dose; Cₘₐₓ showed a linear kinetics over a dose range between 0.45 mg/kg and 2 mg/kg, whereas exposure (AUCt) presented non-linear kinetics over the same dose range.

In some specific aspects, ApTOLL is presented as 1 vial of 7 mg of freeze-dried powder to be reconstituted with 3 mL water to generate an ApTOLL concentrate, which is further diluted with 100 mL 0.9% sodium chloride solution. The resulting solution can be administered intravenously, e.g., via an infusion pump. In some aspects, ApTOLL administration takes place as a single dose. In other aspects, multiple doses are administered. In some aspects, the ApTOLL infusion has a duration of approximately 30 minutes.

In some aspects, when ApTOLL infusion is administered as part of a thrombectomy procedures, the ApTOLL infusion is administered immediately after i.v. thrombolysis comprising rt-PA (recombinant tissue Plasminogen Activator; alteplase) administration, if appropriate, and before thrombectomy.

### IV. CHEMICALLY MODIFIED APTAMERS

Aptamers of the present disclosure (e.g., ApTOLL) can be chemically modified to become extremely stable or can be further truncated to eliminate oligonucleotide sequences that are not important for the interaction with the target or for the correct three-dimensional aptamer structure. The aptamers of the present disclosure can be in the form of unmodified single-stranded DNA (ssDNA) aptamers, e.g., for the treatment of acute ischemic stroke, and other diseases and conditions disclosed herein due to their rapid pharmacokinetics and low toxicity profile. However, to extend, e.g., the therapeutic and/or protective effect of the aptamers of the present disclosure, the aptamers can undergo modifications aimed to increase, e.g., their resistance to degradation by nucleases and/or their half-life in circulation.

In some aspects, an aptamer of the present disclosure (e.g., ApTOLL) comprises at least one chemically modified nucleoside and/or nucleotide. When the aptamers of the present disclosure are chemically modified the aptamers can be referred to as “modified aptamers.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”).

A “nucleotide” refers to a nucleoside including a phosphate group. Modified nucleotides can be synthesized by any useful method, such as, for example, chemically, enzymatically, or recombinantly, to include one or more modified or non-natural nucleosides.

Aptamers of the present disclosure can comprise a region or regions of linked nucleosides. Such regions can have variable backbone linkages. The linkages can be standard phosphodiester linkages, in which case the aptamer would comprise regions of nucleotides.

A modified aptamer disclosed herein can comprise various distinct modifications. In some aspects, the modified aptamer contains one, two, or more (optionally different) nucleoside or nucleotide modifications. In some aspects, a modified aptamer can exhibit one or more desirable properties, e.g., improved thermal or chemical stability, reduced immunogenicity, reduced degradation, increased binding to the TLR-4 target epitope, reduced non-specific binding to other areas of TLR-4 or other molecules, e.g., other Toll-like receptor, as compared to the corresponding unmodified aptamer.

In some aspects, a polynucleotide of the present disclosure (e.g., an aptamer such as ApTOLL) is chemically modified. As used herein in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure (e.g., an aptamer such as ApTOLL) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation. In another aspect, the polynucleotide of the present disclosure (e.g., an aptamer such as ApTOLL) can have a uniform chemical modification of two, three, or four of the same nucleoside type throughout the entire polynucleotide (such as all adenosines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into a polynucleotide of the present disclosure (e.g., an aptamer such as ApTOLL).

In some aspects, the nucleobases, sugar, backbone linkages, or any combination thereof in a polynucleotide of the present disclosure (e.g., an aptamer such as ApTOLL) are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

### 1. Base Modifications

In certain aspects, the chemical modification is at nucleobases in a polynucleotide of the present disclosure (e.g., an aptamer such as ApTOLL). In some aspects, the at least one chemically modified nucleoside is a modified uridine (e.g., pseudouridine (ψ), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide of the present disclosure (e.g., an aptamer such as ApTOLL) is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above.

In some aspects, the polynucleotide of the present disclosure (e.g., an aptamer such as ApTOLL) includes a combination of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or more than 80 modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in a polynucleotide of the present disclosure (e.g., an aptamer such as ApTOLL) are modified nucleobases.

### 2. Backbone Modifications

In some aspects, the polynucleotide of the present disclosure (e.g., an aptamer such as ApTOLL) includes any useful modification to the linkages between the nucleosides. Such linkages, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

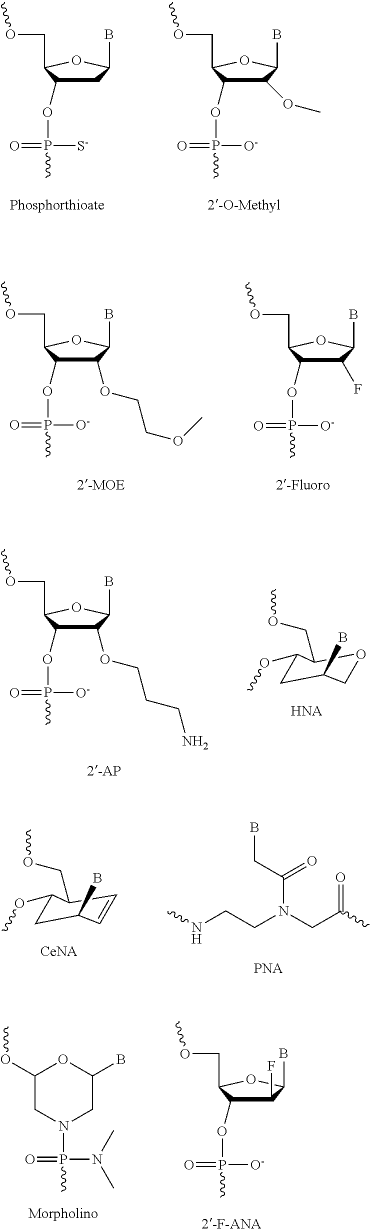

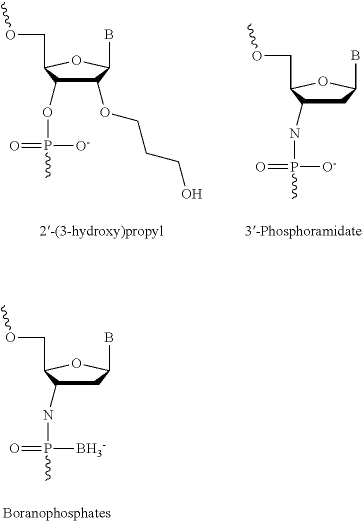

In some aspects, the presence of a backbone linkage disclosed above increases the stability (e.g., thermal stability) and/or resistance to degradation (e.g., enzyme degradation) of a polynucleotide of the present disclosure (e.g., an aptamer such as ApTOLL).

In some aspects, the stability and/or resistance to degradation (e.g, degradation by nucleases) increases by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in the modified polynucleotide of the present disclosure (e.g., an aptamer) compared to a corresponding polynucleotide without the modification (reference or control aptamer).

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a polynucleotide of the present disclosure (e.g., an aptamer such as ApTOLL) are modified (e.g., all of them are phosphorothioate).

In some aspects, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or more than 80 backbone linkages in a polynucleotide of the present disclosure (e.g., an aptamer such as ApTOLL) are modified (e.g., phosphorothioate).

In some aspects, the backbone comprises linkages selected from the group consisting of phosphodiester linkage, phosphotriesters linkage, methylphosphonate linkage, phosphoramidate linkage, phosphorothioate linkage, and combinations thereof.

### 3. Sugar Modifications

The modified nucleosides and nucleotides which can be incorporated into a polynucleotide of the present disclosure (e.g., an aptamer such as ApTOLL), can be modified on the sugar of the nucleic acid. Thus, in some aspects, the aptamer of the present disclosure (e.g., ApTOLL) comprises at least one nucleoside analog (e.g., a nucleoside with a sugar modification).

In some aspects, the sugar modification increases the affinity of the binding of a polynucleotide of the present disclosure (e.g., an aptamer such as ApTOLL) to its target epitope. Incorporating affinity-enhancing nucleotide analogues in the polynucleotide of the present disclosure (e.g., an aptamer such as ApTOLL), such as LNA or 2′-substituted sugars can allow the length of the polynucleotide of the present disclosure (e.g., an aptamer such as ApTOLL) to be reduced, and also can reduce the upper limit of the size a polynucleotide of the present disclosure (e.g., an aptamer such as ApTOLL) before non-specific or aberrant binding takes place.

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% of the nucleotides in a polynucleotide of the present disclosure (e.g., an aptamer such as ApTOLL) contain sugar modifications (e.g., LNA).

In some aspects, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or more than 80 nucleotide units in a polynucleotide of the present disclosure (e.g., an aptamer such as ApTOLL) are sugar modified (e.g., LNA).

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleoside analogues present in a polynucleotide of the present disclosure (e.g., an aptamer such as ApTOLL) comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof.

In some aspects, nucleoside analogs present in a polynucleotide of the present disclosure (e.g., an aptamer such as ApTOLL) comprise Locked Nucleic Acid (LNA); 2′-O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleoside (cEt), 2′-O-methyl nucleic acid (2′-OMe), 2′-O— methoxyethyl nucleic acid (2′-MOE), or any combination thereof.

In some aspects, a polynucleotide of the present disclosure (e.g., an aptamer such as ApTOLL) can comprise both modified RNA nucleotide analogues (e.g., LNA) and DNA units. See, e.g., U.S. Pat. Nos. 8,404,649; 8,580,756; 8,163,708; 9,034,837; all of which are herein incorporated by reference in their entireties.

### V. METHODS OF MANUFACTURE AND FORMULATION

The present disclosure also provides methods of making the aptamers of the present disclosure (e.g., ApTOLL). In general, aptamers of the present disclosure can be obtained used the methods disclosed in U.S. Pat. No. 10,196,642, and synthesized using methods described therein or method generally known in the art.

The production of the aptamer of the present disclosure (e.g., ApTOLL) can be carried out following conventional methods in the art. Non-limiting examples of techniques for the production of aptamers include enzymatic techniques, such as transcription, recombinant expression systems and standard solid phase (or solution phase) chemical synthesis, all commercially available. When appropriate, for example, in the event that the aptamer of the present disclosure comprises nucleic acid variants such as those described above, nucleotide analogues such as analogues having chemically modified bases or sugars, backbone modifications, etc., the aptamer of the invention can be produced by means of chemical synthesis. Alternatively, recombinant expression can be the technique preferred for the production of aptamers of the present disclosure when the aptamers have, e.g., a length of 200 nucleotides or more. The aptamers produced by or any of the preceding techniques can optionally be purified by methods that are well known in the art.

As used herein, the term “synthesizing” refers to the assembling the aptamer using polynucleotide synthesis methods known in the art. The term synthesizing also encompasses the assembly of conjugates or complexes that comprise an aptamer of the present disclosure (e.g., ApTOLL) and at least one biological active molecule (e.g., a small molecule drug covalently or non-covalently attached to the aptamer). For example, peptide or small molecule components can be prepared recombinantly, chemically, or enzymatically and subsequently conjugated to the aptamer (e.g., ApTOLL) in one or more synthesis steps (e.g., conjugation of a linker to an aptamer of the present disclosure followed by conjugation of a small molecule to the linker). In some aspects, each one of the components of a conjugate or complex comprising at least one aptamer of present disclosure (e.g., ApTOLL) can be prepared using methods known in the art, e.g., recombinant protein production, solid phase peptide or nucleic acid synthesis, chemical synthesis, enzymatic synthesis, or any combination thereof, and the resulting components can be conjugated using chemical and/or enzymatic methods known in the art.

The aptamers of the present disclosure (e.g. ApTOLL) can be purified, e.g., via filtration, to remove contaminants. In some aspects, the manufacture of the aptamers of the present disclosure (e.g., ApTOLL) comprise lyophilization or any other form of dry storage suitable for reconstitution. In some aspects, the preparation of the aptamer in a dry form takes place after combination of the aptamer (e.g., ApTOLL) with a biologically active molecule (e.g., a small molecule drug), i.e., both therapeutic agents can be co-lyophilized.

In some aspects, the method of preparing a composition comprising an aptamer of the present disclosure (e.g., ApTOLL) with a biologically active molecule (e.g., a small molecule drug) comprises mixing the aptamer with the biologically active molecule (e.g., a small molecule drug) in solution. In some aspects, after combination of the aptamer (e.g., ApTOLL) and the biologically active molecule (e.g., a small molecule drug) in solution, the resulting solution is lyophilized or dried. In some aspects, the combination of the aptamer (e.g., ApTOLL) and the biologically active molecule (e.g., a small molecule drug) is conducted in dry form.

In some aspects, the aptamers of the present disclosure (e.g., ApTOLL) can be purified, e.g., to remove contaminants and/or to generate an uniform population of aptamers.

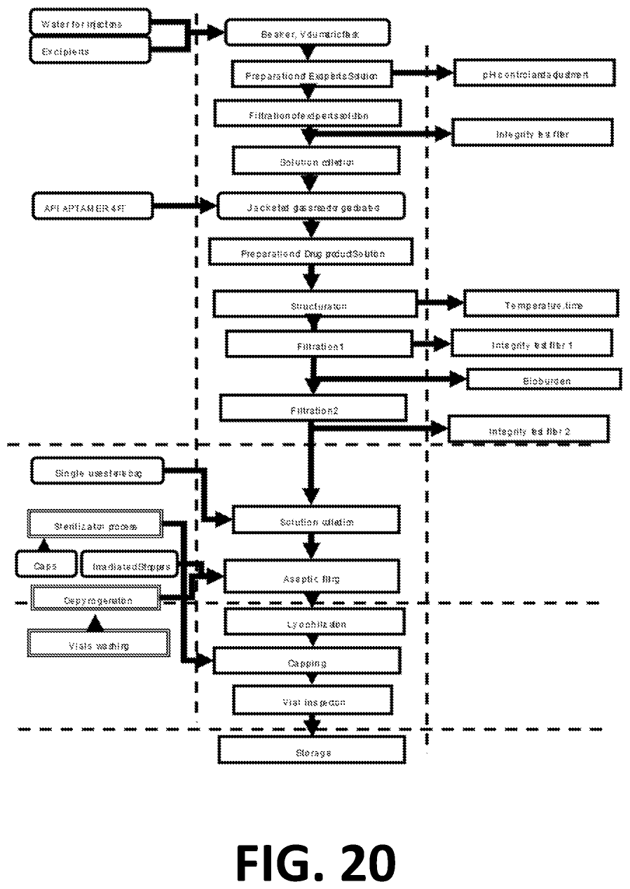

The present disclosure also provides formulations comprising aptamers of the present disclosure, e.g., ApTOLL. The aptamers of the present disclosure can be formulated according to the method depicted schematically in FIG. 20. Aptamer API (Active Pharmaceutical Ingredient) is combined with a solution comprising previously filtered excipients. After a structuration stage, the solution comprising aptamer (e.g., ApTOLL) and excipients is subject to two filtration steps, transferred to vials, and lyophilized. The structuration step is a critical step in the preparation of the aptamer (e.g., ApTOLL). The structuration process comprises dissolving the aptamer in an appropriate solvent. In some aspects, the solvent comprises a divalent ion. In some aspects, the divalent ion is Mg²⁺. In some aspects, the solvent is phosphate buffered saline (PBS) comprising MgCl₂. In some aspects, the solvent is PBS comprising 1 mM MgCl₂. After the aptamer (e.g., ApTOLL) has been dissolved, it is heated up to a denaturing temperature (e.g., 95° C.) for a short period of time (e.g., approximately 10 minutes) followed by rapid cooling (e.g., by transfer to ice, e.g., during approximately 10 minutes). In some aspects, the aptamer (e.g., ApTOLL) is not functional in the absence of the heating and cooling steps.

After synthesis, aptamers of the present disclosure (e.g., ApTOLL) are linear. Increasing the temperature fully linearizes the aptamer, whereas the subsequent cooling down correctly folds the aptamer, resulting in a functional aptamer. In some aspects, the aptamers of the present disclosure (e.g., ApTOLL) are not functional if the heating and cooling steps are not conducted in the presence of a divalent ion, e.g., Mg′. In a particular aspect of the present disclosure, the aptamers of the present disclosure (e.g., ApTOLL) are not therapeutically functional unless they have been dissolved in a buffer containing Mg′ (e.g., 1 mM MgCl₂), heated at 95° C. for 10 minutes, and subsequently cooled at 0° C. in ice for 10 minutes.

The process of manufacture of the aptamers of the presence disclosure (e.g., ApTOLL) comprises two lyophilization steps. In a first step, the structured aptamer (e.g., an aptamer of the present disclosure in PBS is lyophilized. The lyophilized aptamer (e.g., ApTOLL) is redisolved in a buffer, e.g., PBS, and relyophilized. The second lyophilization increases the stability of the aptamer of the present disclosure (e.g., ApTOLL) with respect to the same aptamer undergoing a single lyophilization step.

In some aspects, the aptamers of the present disclosure (e.g., ApTOLL) are formulated in doses comprising 7 mg of aptamer, e.g., structured and lyophilized aptamer. In other aspects, the aptamers of the present disclosure are formulated in doses comprising at least about 1 mg, at least about 2 mg, at least about 3 mg, at least about 4 mg, at least about 5 mg, at least about 6 mg, at least about 7 mg, at least about 8 mg, at least about 9 mg, or at least about 10 mg of aptamer of the present disclosure (e.g., ApTOLL).

In some aspects, the aptamer of the present disclosure can be formulated, e.g., in nanoparticles such as polymeric nanoparticles, lipid nanoparticles (for examples, liposomes or micelles), or metal nanoparticles, comprising the aptamers of the present disclosure covalently or non-covalently attached to the nanoparticle (e.g., encapsulated in the nanoparticle). See, e.g., U.S. Pat. No. 10,196,642, which is herein incorporated by reference in its entirety.

As described above, the aptamers of the present disclosure can be covalently or non-covalently attached to a biologically active molecule and/or to a nanoparticle (e.g., a formed nanoparticle or a component of a nanoparticle). Covalent attachment between an aptamer of the present disclosure (e.g., ApTOLL) and a biologically active molecule and/or a nanoparticle can be carried out by means of conjugation techniques that are well-known by the person skilled in the art. The result is a covalent bond between the aptamer of the present disclosure and a biologically active molecule and/or to a nanoparticle or its components. The conjugation can involve binding of primary amines of the 3′ or 5′ ends of the aptamer of the present disclosure to the functional group during chemical synthesis of the aptamer.

Conjugation can also be done by means of conventional cross-linking reactions, having the advantage of the much greater chemical reactivity of primary alkyl-amine labels with respect to the aryl amines of the nucleotides themselves. Methods of conjugation are well-known in the art and are based on the use of cross-linking reagents. The cross-linking reagents contain at least two reactive groups which target groups such as primary amines, sulfhydryls, aldehydes, carboxyls, hydroxyls, azides, and so on and so forth, in the biologically active molecule and/or nanoparticle to be conjugated to an aptamer of the present disclosure.

The cross-linking agents differ in their chemical specificity, spacer arm length, spacer arm composition, cleavage spacer arm, and structure. For example, conjugation of biologically active molecules and/or nanoparticles or their components to aptamer of the present disclosure can be carried out directly or through a linking moiety, through one or more non-functional groups in the aptamer and/or the functional group, such as amine, carboxyl, phenyl, thiol or hydroxyl groups. More selective bonds can be achieved by means of the use of a heterobifunctional linker. It is possible to use conventional linkers, such as diisocyanates, diisothiocyanates, bis (hydroxysuccinimide) esters, carbodiimides, maleimide-hydroxysuccinimide esters, glutaraldehyde and the like, or hydrazines and hydrazides, such as 4-(4-N-maleimidophenyl) butyric acid hydrazide (MPBH).

In some aspects, conjugation can take place subsequently to the generation of the aptamer of the present disclosure by recombinant or enzymatic methods.

In some aspects, the aptamers of the present disclosure (e.g., ApTOLL) are formulated in vials, wherein each dose vial comprises about 0.5, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10 mg of aptamer of the present disclosure (e.g., ApTOLL) per vial. In one specific aspect, each dose vial comprises 7 mg of aptamer of the present disclosure (e.g., ApTOLL) per vial. In some aspects, the content of the vials is lyophilized aptamer of the present disclosure (e.g., ApTOLL).

### VI. PHARMACEUTICAL COMPOSITIONS

The present disclosure also provides pharmaceutical compositions comprising one or more aptamers of the present disclosure (e.g., ApTOLL) that are suitable for administration to a subject according to the methods disclosed herein (e.g., methods to any of the diseases or conditions disclosed herein, e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke).

The pharmaceutical compositions generally comprise one or more aptamers of the present disclosure (e.g., ApTOLL), having the desired degree of purity, and a pharmaceutically-acceptable excipient or carrier in a form suitable for administration to a subject. Pharmaceutically acceptable excipients or carriers are determined in part by the particular composition being administered, as well as by the particular method used to administer the composition. Accordingly, there is a wide variety of suitable formulations of pharmaceutical compositions comprising one or more aptamers of the present disclosure (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration.

In some aspects, the pharmaceutical composition comprises one or more aptamers of the present disclosure (e.g., ApTOLL). In certain aspects, the aptamers of the present disclosure (e.g., ApTOLL) are co-administered with of one or more additional therapeutic agents, in a pharmaceutically acceptable carrier, and/or a surgical procedure (e.g., thrombectomy in the case of myocardial infarction). In some aspects, the pharmaceutical composition comprising the aptamers of the present disclosure (e.g., ApTOLL) is administered prior to administration of the additional therapeutic agent(s), and/or a surgical procedure (e.g., thrombectomy in the case of myocardial infarction).

In other aspects, the pharmaceutical composition comprising the aptamers of the present disclosure (e.g., ApTOLL) is administered after the administration of the additional therapeutic agent(s), and/or a surgical procedure (e.g., thrombectomy in the case of myocardial infarction). In further aspects, the pharmaceutical composition comprising the aptamers of the present disclosure (e.g., ApTOLL) is administered concurrently with the additional therapeutic agent(s), and/or a surgical procedure (e.g., thrombectomy in the case of myocardial infarction).

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the aptamers of the present disclosure, use thereof in the compositions is contemplated.

Supplementary therapeutic agents suitable for the treatment or prevention (e.g., suppression, inhibition, or delay) of any of the diseases or conditions disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke), or suitable for the improvement of the homeostasis of a subject who is suffering, who has suffered, or who is at the risk of suffering any of the diseases or conditions disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke), can also be incorporated into the compositions of the present disclosure.

Typically, a pharmaceutical composition is formulated to be compatible with its intended route of administration. The aptamers of the present disclosure (e.g., ApTOLL) can be administered, e.g., by parenteral, topical, intravenous, oral, subcutaneous, intraarterial, intradermal, transdermal, rectal, intracranial, intraperitoneal, intranasal, or intramuscular route or as inhalants.

In certain aspects, the pharmaceutical composition comprising aptamers of the present disclosure (e.g., ApTOLL) is administered intravenously or intraarterially, e.g. by injection. The aptamer described herein (e.g., ApTOLL) can optionally be administered in combination with other therapeutic agents that are at least partly effective in treating any of the diseases or conditions disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke), for which the aptamers described herein (e.g., ApTOLL) are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous or intraarterial administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Pharmaceutical compositions of the present disclosure can be sterilized by conventional, well known sterilization techniques. Aqueous solutions can be packaged for use or filtered under aseptic conditions and lyophilized, the lyophilized preparation being combined with a sterile aqueous solution prior to administration.

Sterile injectable solutions can be prepared by incorporating the aptamers of the present disclosure (e.g., ApTOLL) in an effective amount and in an appropriate solvent with one or a combination of ingredients enumerated herein, as desired. Generally, dispersions are prepared by incorporating the aptamers of the present disclosure (e.g., ApTOLL) into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The aptamers described herein (e.g., ApTOLL) can be administered in the form of a depot injection or implant preparation which can be formulated in such a manner to permit a sustained or pulsatile release of the aptamers of the present disclosure.

Systemic administration of compositions comprising aptamers described herein (e.g., ApTOLL) can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising aptamers of the present disclosure (e.g., ApTOLL) is administered intravenously or intraarterially into a subject that would benefit from the pharmaceutical composition. In certain other aspects, the composition is administered to the lymphatic system, e.g., by intralymphatic injection, intranodal injection (see e.g., Senti et al., PNAS 105(46): 17908 (2008)), intramuscular injection, intraperitoneal, or subcutaneous administration.

In certain aspects, the pharmaceutical composition comprising aptamer of the present disclosure (e.g., ApTOLL) is administered as a liquid suspension. In certain aspects, the pharmaceutical composition is administered as a formulation that is capable of forming a depot following administration. In certain preferred aspects, the depot slowly releases the aptamers into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the aptamers described herein (e.g., ApTOLL) and optionally a pharmaceutically active or therapeutic agent. The therapeutic agent can be, e.g., a biological agent (e.g., a peptide or nucleic acid), a small molecule agent, or a combination thereof.

Dosage forms are provided that comprise aptamers (e.g., ApTOLL) or pharmaceutical compositions described herein for use according to the methods disclosed herein. In some aspects, the dosage form is formulated as a liquid suspension for intravenous or intraarterial injection.

An aptamer of the present disclosure (e.g., ApTOLL) or pharmaceutical composition comprising an aptamer of the present disclosure can be used concurrently with other therapies, e.g., drugs and/or surgery. To be specific, the aptamers (e.g., ApTOLL) or pharmaceutical compositions of the present disclosure can be used together with medicaments generally use for the treatment of any of the diseases or conditions disclosed herein (e.g., myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke), or in combination with pharmacologic and/or surgical procedures known in the art used to treat such diseases or conditions (e.g., thrombectomy in the case of myocardial infarction).

### VII. KITS

The present disclosure also provides kits, or products of manufacture, comprising a an aptamer of the present disclosure (e.g., an isolated aptamer of the present disclosure or an aptamer of the present disclosure conjugated or complexed to a biologically active molecule, such as ApTOLL) and optionally instructions for use according to the methods of the present disclosure.

In some aspects, the kit or product of manufacture comprises a pharmaceutical composition of the present disclosure, which comprises at least one aptamer of the present disclosure (e.g., ApTOLL), in one or more containers, and optionally instructions for use according to the methods of the present disclosure.

In some aspects, the kit or product of manufacture comprises an aptamer of the present disclosure (e.g., ApTOLL), or a pharmaceutical composition of the present disclosure and a brochure. In some aspects, the kit or product of manufacture comprises an aptamer of the present disclosure (e.g., ApTOLL), or a pharmaceutical composition of the present disclosure and instructions for use. One skilled in the art will readily recognize that an aptamer (e.g., ApTOLL) or a pharmaceutical composition of the present disclosure, or combinations thereof, can be readily incorporated into one of the established kit formats which are well known in the art.

In some aspects, the kit or product of manufacture comprises an aptamer of the present disclosure (e.g., ApTOLL) in dry form in a container (e.g., a glass vial), and optionally a vial with a solvent suitable to hydrate the aptamer, and optionally instructions for use of the reconstituted product according to the methods disclosed herein. In some aspects, the kit or product of manufacture further comprises at least one additional container (e.g., a glass vial) comprising a biologically active molecule (e.g., a second TLR-4 antagonist).

One skilled in the art will readily recognize that the aptamers of the present disclosure (e.g., ApTOLL), pharmaceutical compositions comprising the aptamers of the present disclosure (e.g., ApTOLL), or combinations thereof can be readily incorporated into one of the established kit formats which are well known in the art.

In some aspects, the kit comprises reagent to conjugate a biologically active molecule to an aptamer of the present disclosure (e.g., ApTOLL), instructions to conduct the conjugation, and instructions to use the conjugate according to the methods of the present disclosure.

In some aspects, the kit comprises a biologically active molecule and an aptamer of the present disclosure (e.g., ApTOLL), instructions to conduct to admix them to form a complex, and instructions to use the resulting complex according to the methods of the present disclosure.

In some aspects, the kit or product of manufacture comprises aptamers of the present disclosure (e.g., ApTOLL) in solution, and instructions for use according to the methods of the present disclosure. In some aspects, the kit or product of manufacture comprises an aptamer of the present disclosure (e.g., ApTOLL) in dry form, and instructions for use (e.g., instructions for reconstitution and administration according to the methods disclosed herein).

### VIII. EMBODIMENTS

E1. A method of treating a TLR-4 mediated disease or condition in a subject in need thereof comprising administering to the subject at least one dose of a nucleic acid aptamer 40 to 80 nucleobases in length, wherein the aptamer binds to an epitope on the extracellular domain of TLR-4, and wherein binding of the aptamer to the epitope reduces and/or inhibits TLR-4 activation.

E2. The method of embodiment E1, further comprising administering an additional treatment or a combination thereof.

E3. The method of embodiment E2, wherein the additional treatment is a second TLR-4 antagonist.

E4. The method of embodiment E3, wherein the additional treatment is a surgical intervention.

E5. The method of embodiment E2, wherein the additional treatment comprises the administration of an anti-inflammatory agent, a nucleic acid, a peptide, or a combination thereof.

E6. The method of embodiment E5, wherein the peptide comprises an antibody or an antigen-binding fragment thereof.

E7. The method of embodiment E5, wherein the nucleic acid comprises an antisense oligonucleotide, an antimir, a siRNA, or an shRNA.

E8. The method of embodiment E1, wherein the nucleic acid aptamer comprises a sequence at least 70% identical to SEQ ID: 1, 2, 3, or 4, or a combination thereof.

E9. The method of embodiment E1, wherein the nucleic acid aptamer further comprises a biologically active molecule covalently or non-covalently attached to the aptamer.

E10. The method of embodiment E1, wherein the nucleic acid aptamer cross-competes with or binds to the same TLR-4 epitope as a nucleic acid aptamer of SEQ ID: 1, 2, 3, or 4.

E11. The method of embodiment E1, wherein the nucleic acid aptamer cross-competes with or binds to an epitope that overlaps the TLR-4 epitope recognized by a nucleic acid aptamer of SEQ ID: 1, 2, 3, or 4.

E12. The method of embodiment E1, wherein the nucleic acid aptamer is administered in a dose regimen comprising multiple doses.

E13. The method of embodiment E12, wherein the multiple doses are administered concurrently, consecutively, or a combination thereof.

E14. The method of embodiment E12, wherein the multiple doses comprise two, three, four, or five doses.

E15. The method of embodiment E1, wherein each dose comprises between 0.007 and 0.45 mg/kg of nucleic acid aptamer.

E16. The method of embodiment E1, wherein the nucleic acid aptamer is administered intravenously, intraarterially, or intraperitoneally.

E17. The method of embodiment E1, wherein the TLR-4 mediated disease or condition is an ischemic disease or condition.

E18. The method of embodiment E17, wherein the ischemic condition is myocardial infarction or ischemic stroke.

E19. The method of embodiment E1, wherein the TLR-4 mediated disease or condition is a hemorrhagic condition.

E20. The method of embodiment E19, wherein the hemorrhagic condition is hemorrhagic stroke or hemorrhagic transformation.

E21. The method of embodiment E1, wherein the TLR-4 mediated disease or condition is a neuromuscular disease or condition.

E22. The method of embodiment E21, wherein the neuromuscular disease or condition is a neurodegenerative disease or condition.

E23. The method of embodiment E22, wherein the neurodegenerative disease or condition is multiple sclerosis.

E24. A method of ameliorating or improving at least a symptom or sequelae of acute cardiac infarction a subject in need thereof comprising administering an aptamer to the subject during, prior, or immediately after the acute cardiac infarction, wherein

(a) the aptamer has a length between 40 and 100 nucleotides and is selected from the group consisting of SEQ ID NOS: 1, 2, 3, and 4, wherein
(i) the aptamer specifically binds to an epitope on the extracellular domain of TLR-4; and,
(ii) binding of the aptamer to the epitope reduces and/or inhibits TLR-4 activation; or

(b) the aptamer is a functional equivalent variant of the aptamer of (a) having at least 85% sequence identity to SEQ ID NO: 1, 2, 3, or 4, wherein the functionally equivalent variant is derived from SEQ ID NO: 1, 2, 3, or 4, and maintains the capability of specifically binding to and reducing and/or inhibiting TLR-4 activation.

E25 The method of embodiment 24, wherein the administration of the aptamer causes a reduction of infarct area.

E26. The method of embodiment E25, wherein the administration of the aptamer causes a reduction of infarct area of that least 25% compared to control conditions.

E27. The method of embodiment E24, wherein the administration of the aptamer causes a decrease in fibrosis and/or necrosis caused by the acute cardiac infarction.

E28. The method of embodiment E24, wherein the administration of the aptamer results in

(i) improvement in cardiac function;

(ii) reduction of degradation of extracellular matrix;

(iii) improvement in cardiac remodeling;

(iv) preservation in ventricular anatomy;

(v) reduction of progression of the infarction; or

(vi) any combination thereof.

E29. A method of ameliorating or improving at least a symptom or sequelae of a neuromuscular or neurodegenerative disease or condition a subject in need thereof comprising administering an aptamer to the subject during, prior, or after the onset of the neuromuscular or neurodegenerative disease or condition, wherein

(a) the aptamer has a length between 40 and 100 nucleotides and is selected from the group consisting of SEQ ID NOS: 1, 2, 3, and 4, wherein
(i) the aptamer specifically binds to an epitope on the extracellular domain of TLR-4; and,
(ii) binding of the aptamer to the epitope reduces and/or inhibits TLR-4 activation; or

(b) the aptamer is a functional equivalent variant of the aptamer of (a) having at least 85% sequence identity to SEQ ID NO: 1, 2, 3, or 4, wherein the functionally equivalent variant is derived from SEQ ID NO: 1, 2, 3, or 4, and maintains the capability of specifically binding to and reducing and/or inhibiting TLR-4 activation.

E30. The method of embodiment E29, wherein administration of the aptamer causes

(i) reduction in demyelination;

(ii) reduction in axonal damage; or,

(iii) a combination thereof.

E31. The method of embodiment E30, wherein the administration of the aptamer causes an inhibition of demyelination of at least 20-80% compared to control conditions (e.g., administration of placebo).

E32. The method of embodiment E30, wherein the administration of the aptamer causes a reduction in axonal damage of at least 10-30% compared to control conditions (e.g., administration of placebo).

E33. The method of embodiment E29, wherein the neuromuscular or neurodegenerative disease or condition is selected from the group consisting of myocardial infarction, hemorrhagic stroke, hemorrhagic transformation, multiple sclerosis, amyotrophic lateral sclerosis, Parkinson's disease, Huntington's disease, Alzheimer's disease, vascular dementia disease, or ischemic stroke,

E34. The method of embodiments E24 or E29, wherein the aptamer is ApTOLL.

E35. The method of embodiments E24 or E29, wherein the aptamer is administered at a dose range between about 0.5 mg/dose and about 14 mg/dose.

E36. The method of embodiments E24 or E29, wherein the aptamer is administered at a dose range between about 0.007 mg/kg per dose and about 0.2 mg/kg per dose.

E37. The method of embodiments E24 or E29, wherein the aptamer is formulated in PBS (sodium chloride, potassium chloride, disodium hydrogen phosphate dehydrate, and potassium dihydrogen phosphate) pH 7.4, comprising magnesium chloride hexahydrate, and optionally comprising A-trehalose dihydrate.

E38. The method of embodiments E24 or E29, wherein the aptamer is administered intravenously by infusion.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

The contents of all cited references (including literature references, patents, patent applications, and websites) that may be cited throughout this application are hereby expressly incorporated by reference in their entirety for any purpose, as are the references cited therein.

The following examples are offered by way of illustration and not by way of limitation.

### EXAMPLES

Methods for the selection, characterization, and optimization of the aptamers of the present disclosure, are disclosed in detail in U.S. Pat. No. 10,196,642, which is herein incorporated by reference in its entirety.

### Example 1. In Vitro Primary Pharmacodynamics

Antagonistic Effect of Aptamers Against hTLR-4 Activation

hTLR-4 activation assay was performed on HEK-blue-hTLR4 cells. Ultrapure LPS (0.1 ng/ml) was used as, important aspects related to the mechanism of renal stone formation can also be derived from such studies.

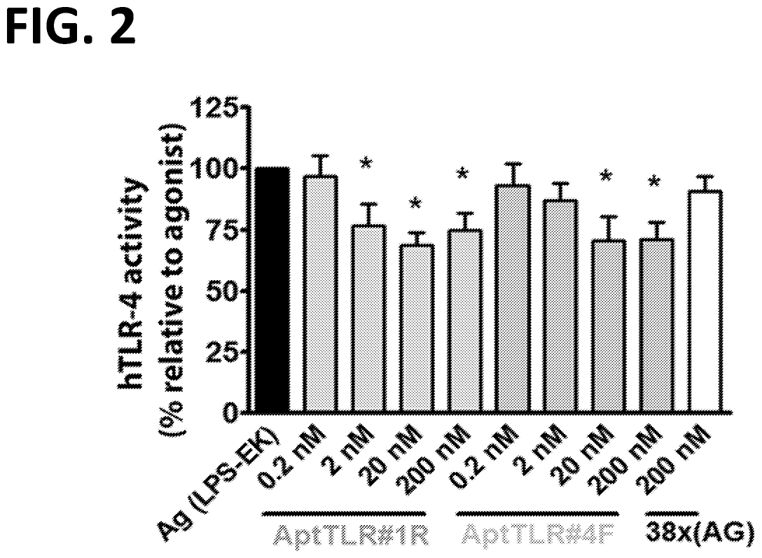

TLR-4 agonist in order to activate cells and a natural LPS antagonist (LPS-RS, 200 ng/ml) was used as positive control of antagonistic activity on hTLR-4. hTLR-4 activation was quantified by the measurement of the secreted embryonic alkaline phosphatase (SEAP) 24 hours after the addition of the ligands to the incubation medium. A poly-AG nucleotide (38×) (38×(AG)) was used as control ssDNA (as a scramble. The 38×(AG), is an oligonucleotide ssDNA, fixed sequence, 38 times A-G. It has been designed in the laboratory, therefore it is a control aptamer without any 3D structure, or with a very limited and unstable structural variety, which does not recognize specifically any target, in fact when it interacts with proteins it does so only by weak loads. Results showed that both ApTLR #1R and ApTLR #4F partially inhibited hTLR-4 activation induced by LPS (FIG. 2). The concentration-response curve showed that maximal antagonistic activity was obtained for the 20 nM concentration (ApTLR #1R) and the 200 nM (ApTLR #4F), showing a 30% of reduction of hTLR-4 activation mediated by LPS. No further effect was observed when increasing concentrations due to the saturation of receptors. No agonistic activity of the aptamers was observed during the assay.

Lead Optimization of Aptamers with hTLR-4 Antagonistic Activity

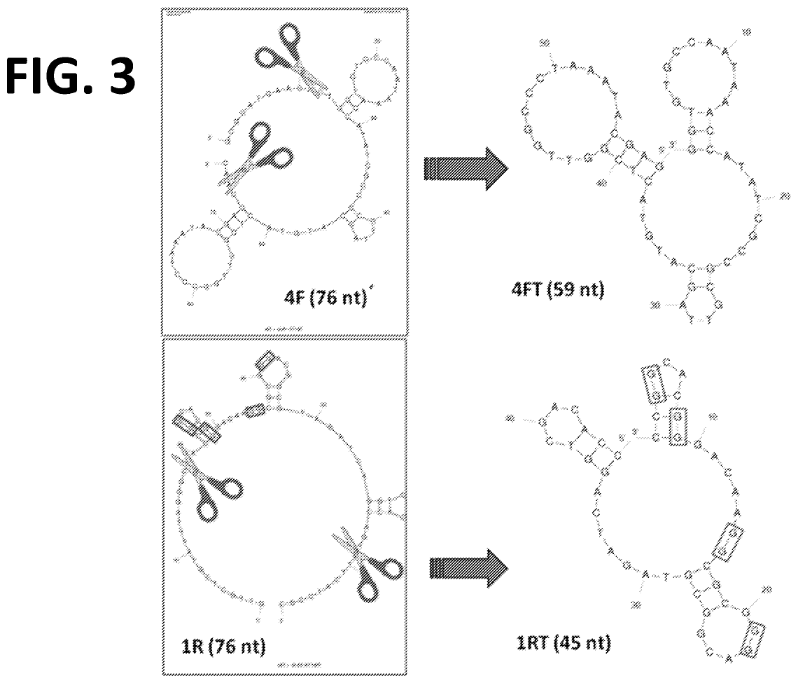

The sequence and derived secondary structure of aptamers ApTLR #1R and ApTLR #4F were modified by deletion of regions located at both ends of each molecule (which neither contribute to the acquisition of the secondary structure nor are expected to affect specific binding properties) in order to improve the bioavailability and body distribution of the molecules. The resulting truncated forms of the aptamers were named ApTLR #1RT and ApTLR #4FT (FIG. 3).

In order to test whether ApTLR #1RT and ApTLR #4FT maintained the same affinity for hTLR-4 shown by the parent molecules, flow cytometry assays were performed using ApTLR #1RT and ApTLR #4FT (20 nM) conjugated with Alexa Fluor 488 and incubated with 293-hTLR4A cells, using HEK-293 cells with no TLR-4 expression as control.

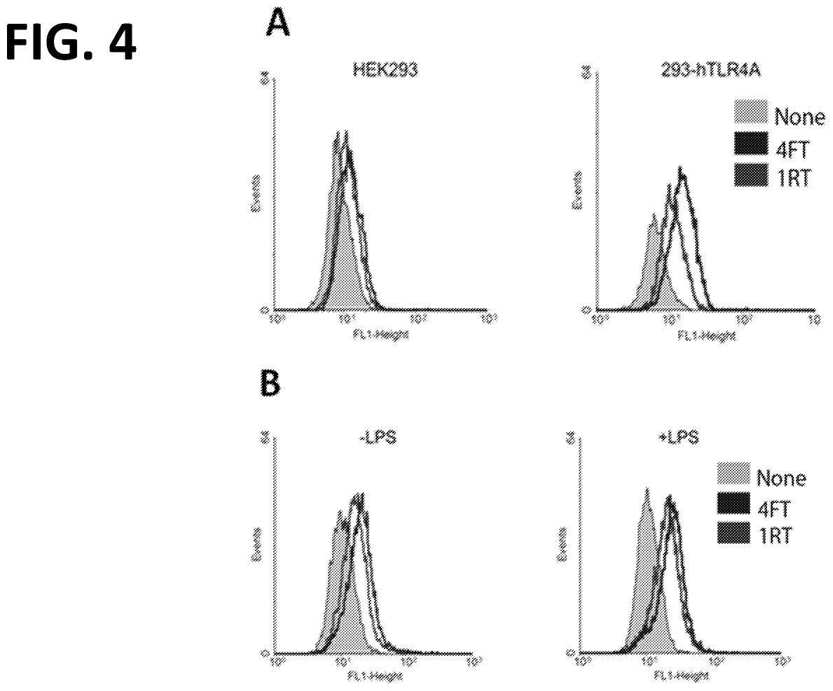

Both aptamers bound 293-hTLR4 cells (FIG. 4, panel A, right panel), but not HEK-293 cells (FIG. 4, panel A, left panel), showing a higher binding affinity for ApTLR #4FT (FIG. 4, panel A, blue line) than ApTLR #1RT (FIG. 4, panel A, red line). When cells were previously activated with LPS, the increase in FL-1 signal was slightly higher in 293-hTLR4A cells (average increase of 9.9) than in HEK-293 cells (average increase of 9.04) (FIG. 4, panel B, left vs right panels).

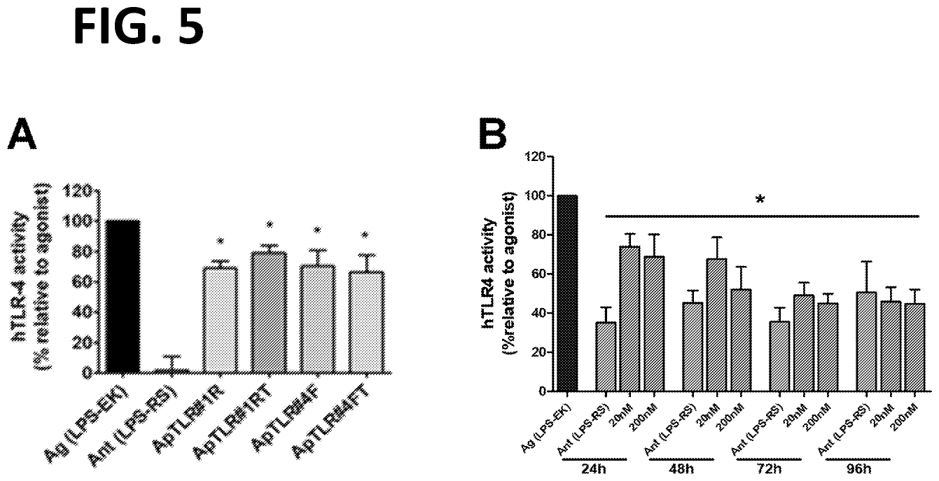

Quantification of the antagonistic activity of the truncated aptamers by the SEAP assay showed that both aptamers maintained the properties of the parent molecules at 20 nM concentration (FIG. 5, panel A). Moreover, in the case of ApTLR #4FT, this inhibitory activity was shown after 96 h of administration (FIG. 5, panel B).

Antagonistic Effect Against hTLR-4 Activation by DAMPs

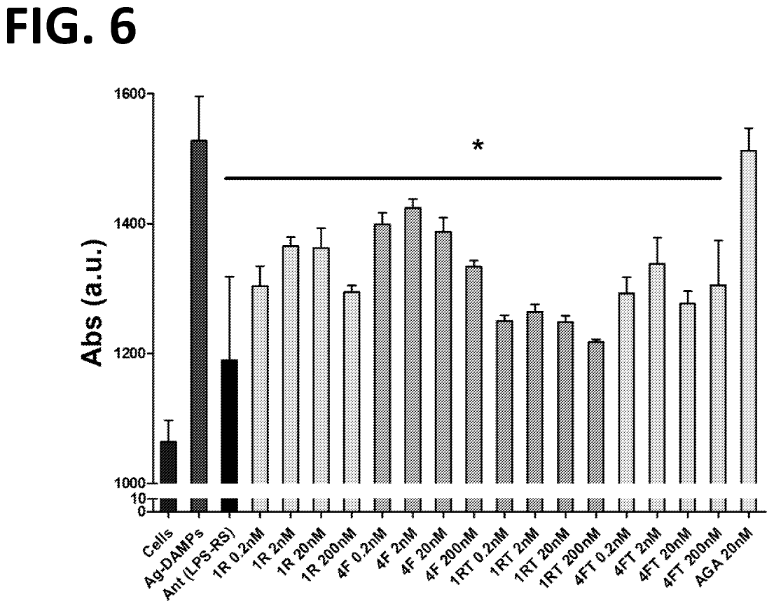

The antagonistic profile of ApTLR #1R, ApTLR #4F and the corresponding truncated forms was also tested against endogenous TLR-4 ligands, reproducing a molecular environment of TLR-4 activation similar to that in the ischemic brain tissue. Endogenous TLR-4 agonists, also known as DAMPs (Damage-Associated Molecular Patterns) are tissue molecules such as heat-shock proteins, nucleic acids, fibronectin or hyaluronan, that are released in the brain parenchyma under damaging conditions. In order to simulate TLR-4 activation by DAMPs, HEK-blue-hTLR4 cells (expressing SEAP in response to TLR-4 activation) were incubated with a HEK-293 cell lysate containing cell-derived DAMPs. In a prior experiment it was determined that a 1:1 dilution of the cell lysate was comparable to 0.2 ng LPS in terms of TLR-4 activation. The cell lysate dilution was added to the incubation medium in the presence or absence of several concentrations of aptamers. All four aptamers partially counteracted hTLR-4 activation induced by DAMPs at all concentrations tested (FIG. 6). AGA (38×AG) was used as control ssDNA (scramble).

Therefore, starting from two candidate aptamers (ApTLR #1R and ApTLR #4F) with confirmed antagonistic activity against TLR-4, optimized truncated forms were generated for additional testing of in vitro and pharmacology. The development of aptamers for the treatment of ischemic stroke focus on ApTLR #4F and ApTLR #4FT for further characterization. A battery of studies aimed to characterize the pharmacodynamic, pharmacokinetic and toxicology properties of both aptamers was initiated in order to identify the best candidate aptamer. Having shown similar pharmacokinetic and toxicology profiles, the pharmacodynamic criteria was used for selection of the leading molecule. In this regard, ApTLR #4FT showed a better dose-response curve of efficacy in the mouse pMCAO model, as well as a greater efficacy in the rat tMCAO model, covering a wider range of ischemic models in vivo. Additionally, the smaller size of ApTLR #4FT pointed towards a better distribution in of the molecule in body compartments, an interesting feature in an indication as ischemic stroke in which one the potential target organs is the brain. Although it is well-known that under ischemic conditions the blood-brain barrier is more permissive than under normal conditions, a smaller molecular size can improve even more the brain distribution following intravenous or intraarterial administration. Together, these evidences pointed towards ApTLR #4FT as the candidate aptamer with better pharmacological profile for the indication of stroke, and ApTLR #4FT (designated ApTOLL) was selected for further development towards its clinical positioning.

### Pharmacodynamic Effect of ApTOLL on Biologically Relevant Inflammatory End-Points

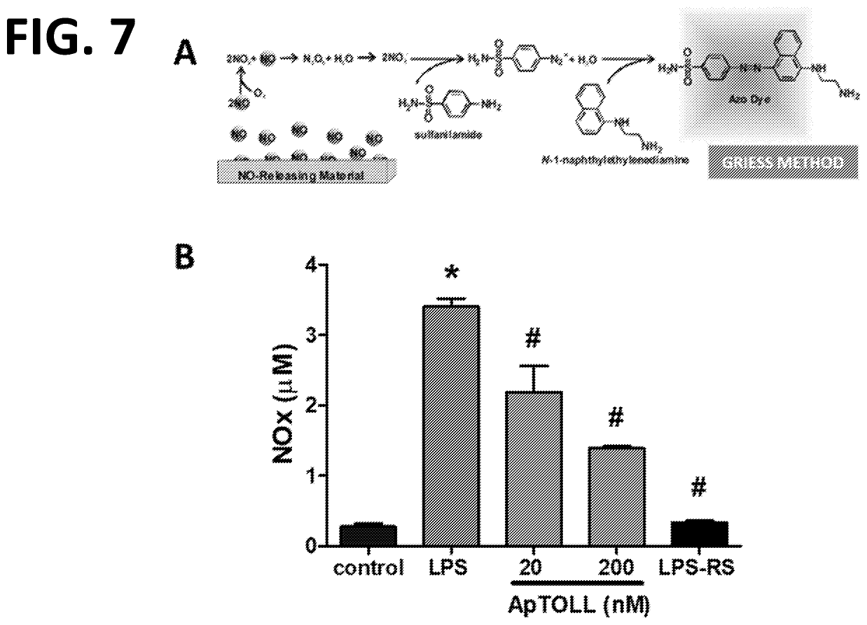

Antagonistic activity of ApTOLL was further confirmed in mice peritoneal macrophages stimulated by LPS (500 ng/ml). ApTOLL (20 nM and 200 nM) was added to the incubation medium 1 hour after LPS, and 24 hours later the concentration of NOx was measured by the Griess reaction (FIG. 7, panel A), as an end-point parameter of the enzymatic activity of the inducible nitric oxide synthase, one of the main target proteins expressed in response to TLR-4 activation. The aptamer induced a reduction of NOx levels in the incubation medium (FIG. 7, panel B).

### In Vitro Binding Characterization

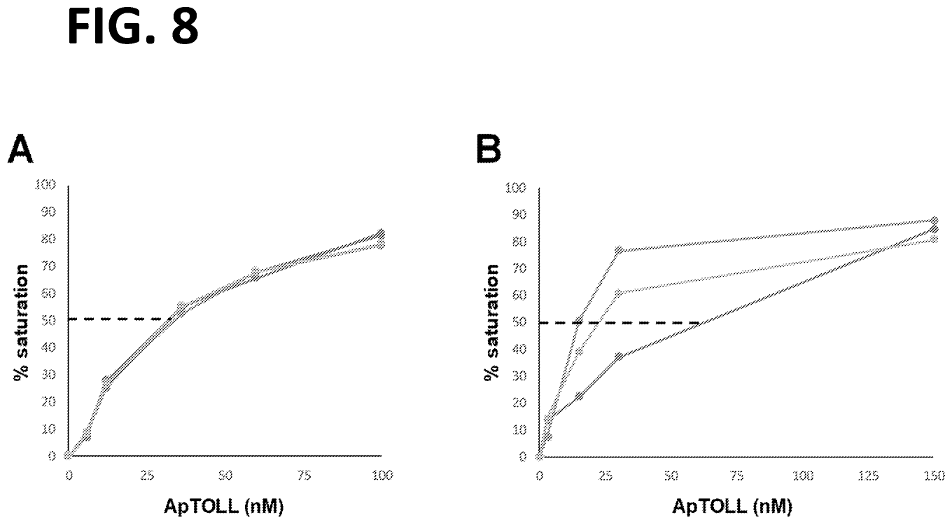

In order to characterize affinity of TLR-4 receptor to ApTOLL, affinity studies were performed in monocytes. For this purpose, cells were obtained from Cynomolgus monkey and human blood samples and incubated in RPMI1640 medium supplemented with 2% FBS (2-4 million/ml cells). ApTOLL-488 (0 to 100 nM) and LPS (50 nM) were added to the medium and the cells were analyzed by flow cytometry. A total of 10.000 viable cells were counted. Propidium iodide staining was carried out to eliminate the non-viable population. Results showed that the Ka (affinity constant) presented values of 30-60 nM in monkeys and human monocytes (FIG. 8).

### Absence of Binding to Other Toll-Like Receptors

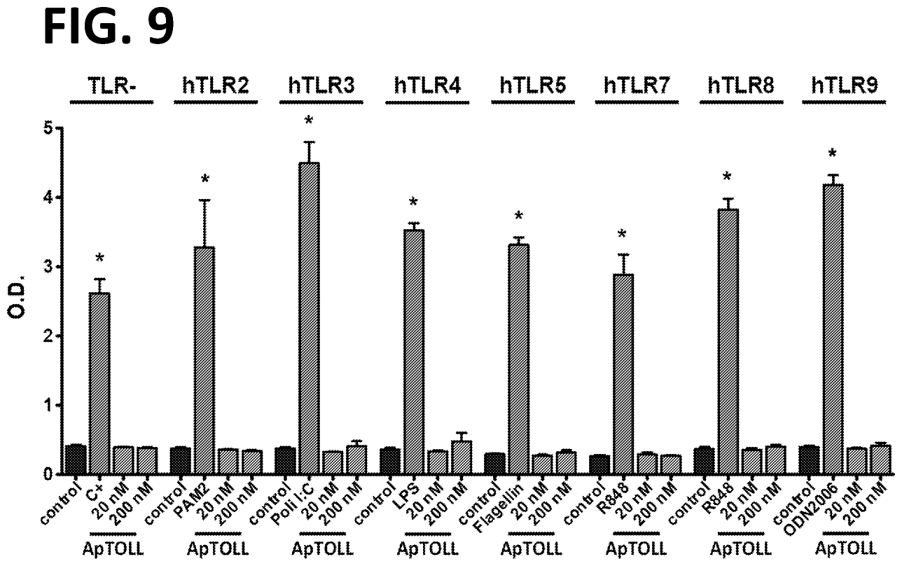

In order to characterize as better as possible the non-agonistic effect of ApTOLL in all TLRs, HumanTLR2,-3-4-5-7-8-and 9 expressing cell lines were incubated with ApTOLL (20 nM and 200 nM) and their corresponding agonists in a specific study. The results showed no agonistic activity of the aptamer in any TLR tested (FIG. 9).

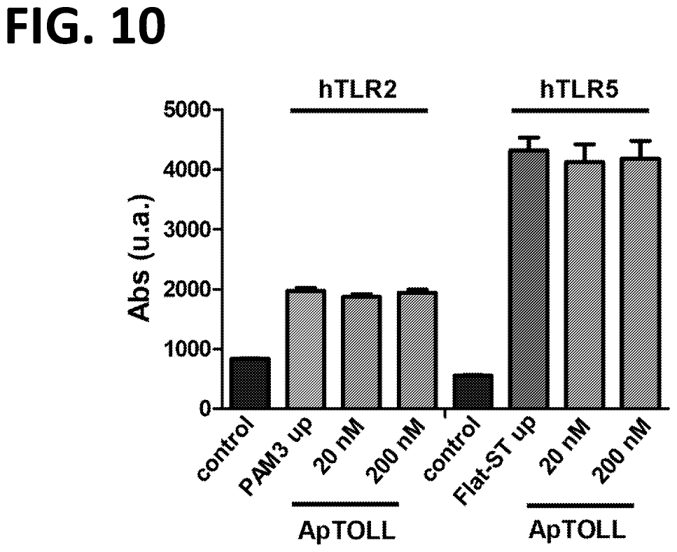

Specificity of ApTOLL against toll-like receptor type-2 (TLR2) and -5 (TLR5) (members of the toll-like receptor family with higher structural and functional homology with TLR-4) was evaluated using cell lines expressing hTLR2 and hTLR5 coupled to the SEAP reporter system. Aptamers showed no interference with hTLR2 and hTLR5 activation by Pam3 and FLAT-ST, respectively, indicating an absence of antagonistic activity on hTLR2 and hTLR5 (FIG. 10). Therefore, ApTOLL did not show any antagonistic activity on these receptors.

### Example 2. In Vivo Primary Pharmacodynamics. Efficacy in Rodent Models of Stroke

The animal models used in the study consisted of:

a) a permanent middle cerebral artery occlusion by ligature (pMCAO) and transient middle cerebral artery occlusion by ligature (tMCAO) in mice (Chen et al. (1986) Stroke 17(4):738-743),

b) transient intraluminal middle cerebral artery occlusion in rats (tMCAO) (Justicia et al. (2001) J Cereb Blood Flow Metab 21(9):1097-1104),

c) and permanent middle cerebral artery occlusion by electrocoagulation in rats and mice (Morancho et al., Neuropathol Appl Neurobiol 2012).

In all models, a unilateral focal ischemic lesion was surgically induced in the brain cortex by permanent or transient middle cerebral artery occlusion (MCAO). To follow the STAIR recommendations of preclinical investigation in stroke (STAIR group: Update of the Stroke Therapy Academic Industry Roundtable Preclinical Recommendations. Stroke 2009, 40(6):2244-50), different approved ischemic models (electrocoagulation, ligature and intraluminal) were performed and the results were reproduced in four independent laboratories. In all experimental groups, animals were anesthetized with 2% isofluorane mixed in 20% O2 and 80% compressed air, body temperature was monitored and stabilized by a thermostatic heating path during the whole procedure and brain injury was assessed by T2-weighted magnetic resonance imaging (T2WI) or by staining of brain sections with 2,3,5-Triphenyltetrazolium Chloride (TTC). Resonance images or TTC-stained brain sections obtained at 24 (pMCAO by ligature, pMCAO by electrocoagulation and tMCAO in rat) or 48 hours (tMCAO in mouse; which in this particular tMCAO model infarct volume can vary between 24 and 48 hours) after occlusion were used for the quantification of infarct size.

2.1. pMCAO by Ligature Mouse Model

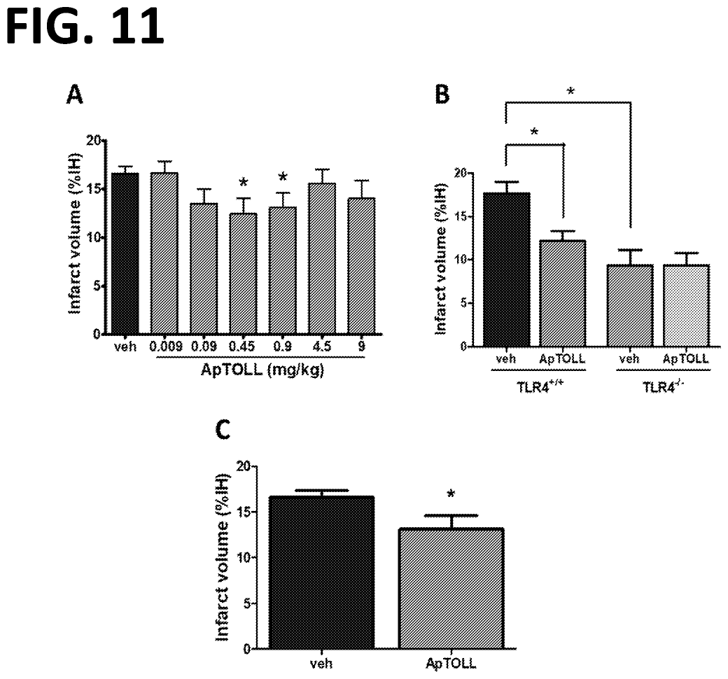

ApTOLL was injected intraperitoneally in wild-type male mice (C57bl/10J) 8-10 weeks old, in a single injection given 10 min after permanent middle cerebral artery occlusion. A dose-response study was performed covering doses from 0.009 mg/kg to 9 mg/kg, a minimum of 9 animals were guaranteed per study-group. Quantification of brain infarct size revealed a protective effect of ApTOLL (26.7% reduction, n=9 per group) (FIG. 11, panel A) at the 0.91 mg/kg dose compared to the vehicle group (n=15). ApTOLL also showed protection at the 0.45 mg/kg dose. The rest of the doses tested showed no statistically significant effect on infarct size.

In order to confirm that the reduction of infarct size induced by ApTOLL was due to TLR-4 antagonism, the aptamer was injected in TLR4 knock-out male mice 8-10 weeks old (C57Bl/10ScNJ, n=4). No protective effect was observed when TLR-4 was absent (FIG. 11, panel B), indicating that TLR-4 inhibition was directly involved in protection mediated by ApTOLL.

Since the intravenous route would be the most likely for administration in human stroke patients, protective effect of ApTOLL, which had been already characterized after intraperitoneal administration, was tested following intravenous injection in the tail or jugular veins. Results showed that protection mediated by ApTOLL was maintained after intravenous injection of a single bolus (0.91 mg/kg; FIG. 11, panel C).

2.2. pMCAO by Electrocoagulation Mouse Model

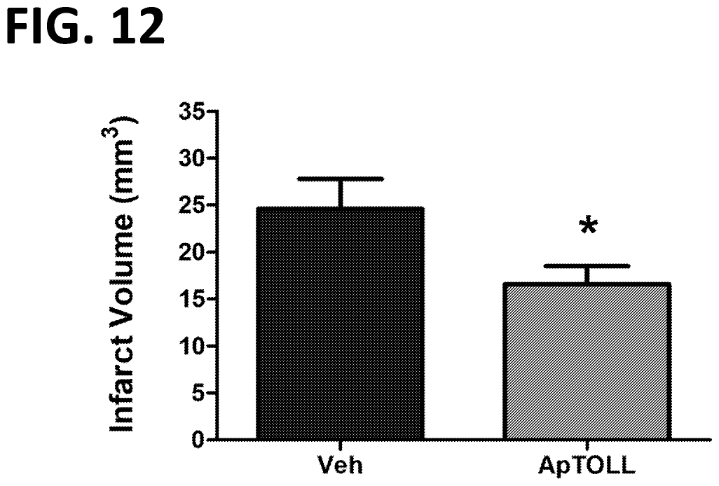

ApTOLL (0.91 mg/kg) or vehicle were injected intraperitoneally in C57bl/6J 8-10 weeks old male mice (n=15), in a single injection given 10 min after permanent middle cerebral artery occlusion. Infarct size was analysed by TTC staining of brain sections at 24 hours after occlusion. Results showed a decrease of 32% in infarct volume in mice treated with ApTOLL vs. vehicle (FIG. 12).

2.3. pMCAO by Electrocoagulation Rat Model. Multiple Administration.

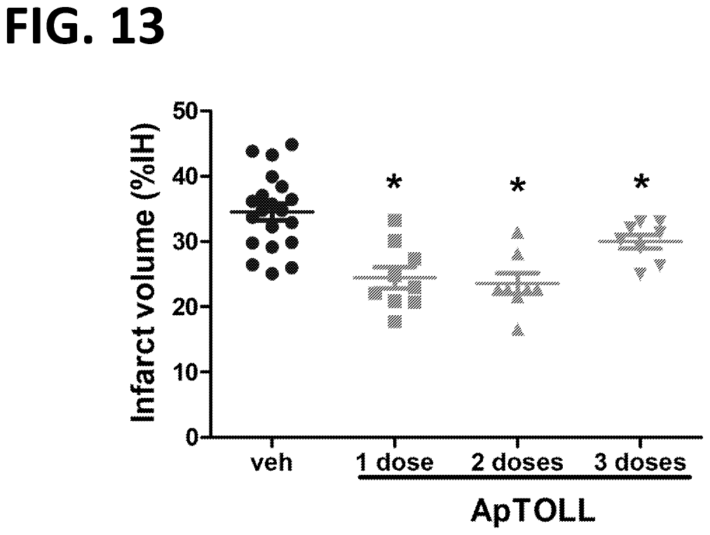

The 0.91 mg/kg ApTOLL dose used in the mouse model was extrapolated to the rat following FDA guidelines for dose extrapolation among species (according to the body surface criterion and correcting for animals' weight) and 0.45 mg/kg were intravenously injected in male Wistar rats 8-10 weeks old 10 min after occlusion (8 animals per group). In this assay, a second and third doses were administered 2 h (10 min+2 h) and 6 h (10 min+2 h+6 h) after occlusion in order to determine the effect of several doses administration. Infarct volume was assessed 48 h after occlusion (FIG. 13).

Results and Conclusions: Administration of 0.45 mg/kg of ApTOLL in rats 10 min after ischemia induced a decreased infarct volume when compared with vehicle-treated animals (32.4% protection, n=8). When ApTOLL was administered twice (10 min and 2 h after occlusion) a reduced final infarct volume was observed as well (35% protection, n=8). Finally, when a third dose was administered, a reduction of 15% was confirmed. These data confirmed the efficacy of ApTOLL in an animal model of permanent ischemia with multiple dose administration and in a different rodent specie (rat). The study was completed with the administration of a fourth dose at 24 h, assessing infarct 48 h after occlusion, and the administration of a fifth dose at 48 h after occlusion and measuring the infarcted area at 72 h after occlusion.

Administration of 0.45 mg/kg of ApTOLL in rats 10 min after ischemia induced a decreased infarct volume when compared with vehicle-treated animals (19% protection, n=8). When multiple doses of ApTOLL were administered, a reduction of the infarct volume was also detected. Two doses administered 10 min and 2 h after occlusion resulted in 21% protection (n=8). Three doses administered 10 min, 2 h, and 6 h after occlusion resulted in 24% protection (n=8). Four doses administered at 10 min, 2 h, 6 h, and 24 h after occlusion resulted in 25% protection (n=8). Five doses administered at 10 min, 2 h, 6 h, 24 h, and 48 h after occlusion resulted in 18% protection, n=8). The term protection refers to the prevention, inhibition, or reduction in the infarct volume, which is an effect or sequela of ischemia.

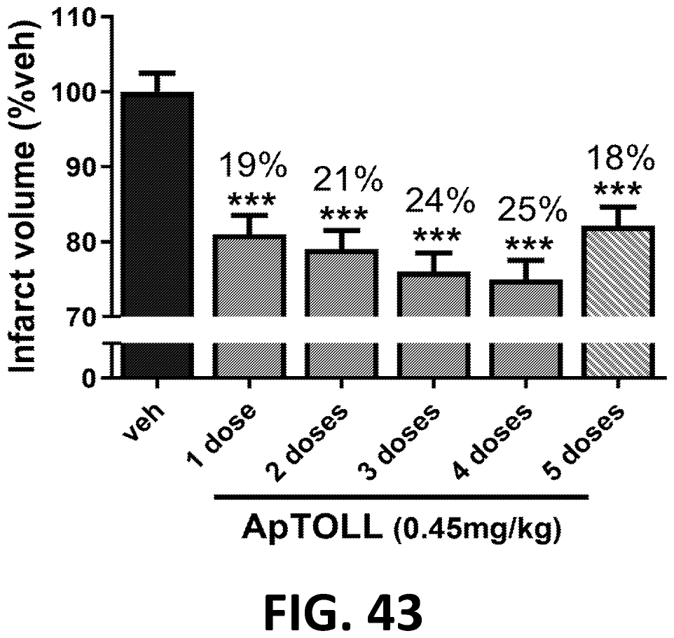

Animals in the groups receiving one, two, three or four doses were euthanized 48 h after pMCAO. Animals in the group receiving 5 doses were euthanized 72 h after pMCAO. All ApTOLL-treated groups were compared with their respective vehicle-treated group (FIG. 43). Therefore, ApTOLL induces a decrease in the infarct volume when administered in multiple doses after the ischemic event, e.g., one, two, three, four or five doses, administered over a period of time of up to 48 after the ischemic event.

2.4. tMCAO Rat Model

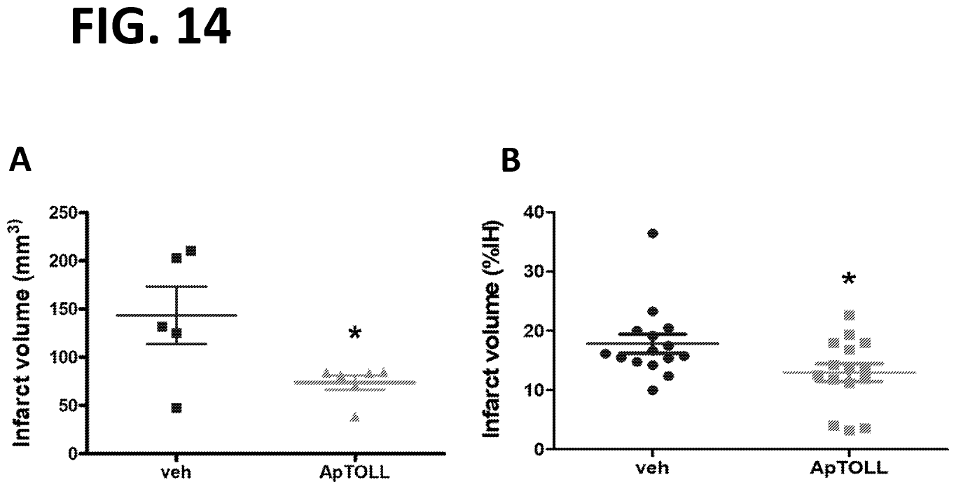

Male Wistar 8-10 weeks old rats (5 animals per group) were used in this study. Ten minutes after surgery rats were administrated with 0.45 mg/kg of ApTOLL or vehicle intravenously, led to reduce final infarct volume at 24 hours after induction of stroke (FIG. 14, panel A) as compared to rats that received vehicle. These data confirmed the efficacy of ApTOLL in a different animal model of ischemia-reperfusion.

Moreover, male Sprague Dawley 8-10 weeks old rats (15 animals per group) were used to reproduce this result. The ischemic procedure and the treatment were the same as described before but the assay was performed in an independent laboratory. Results showed a decrease in infarct volume in rats which received ApTOLL when compared with vehicle-treated animals (FIG. 14, panel B).

2.5. Characterization of the Time Window of Protection of ApTOLL after Stroke Onset

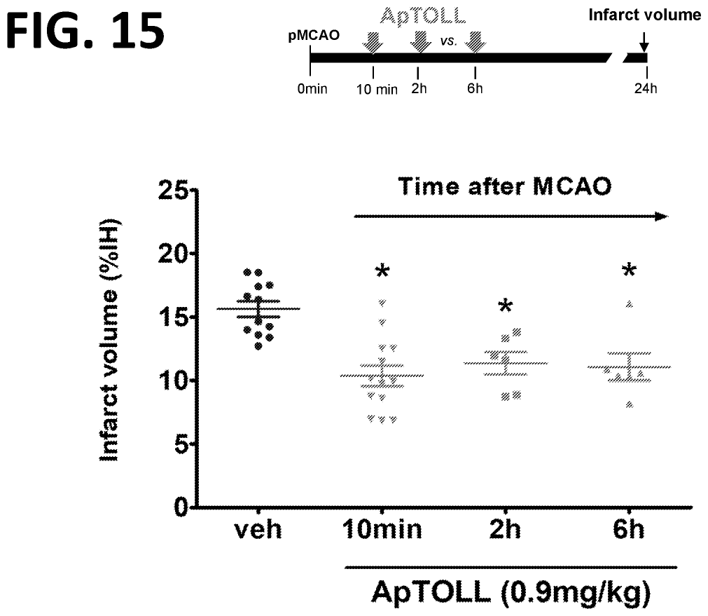

Studies in mice were performed in C57Bl/6 male strain, 8-10 weeks old (8 animals per group), which were subjected to permanent middle cerebral artery occlusion by ligature. Protection mediated by ApTOLL (0.91 mg/kg) was maintained when given intravenously up to 6 hours after pMCAO (FIG. 15), thus extending the therapeutic window of the only pharmacologic therapy for acute ischemic stroke treatment (r-tPA). The time window of protection may extend beyond 6 hours.

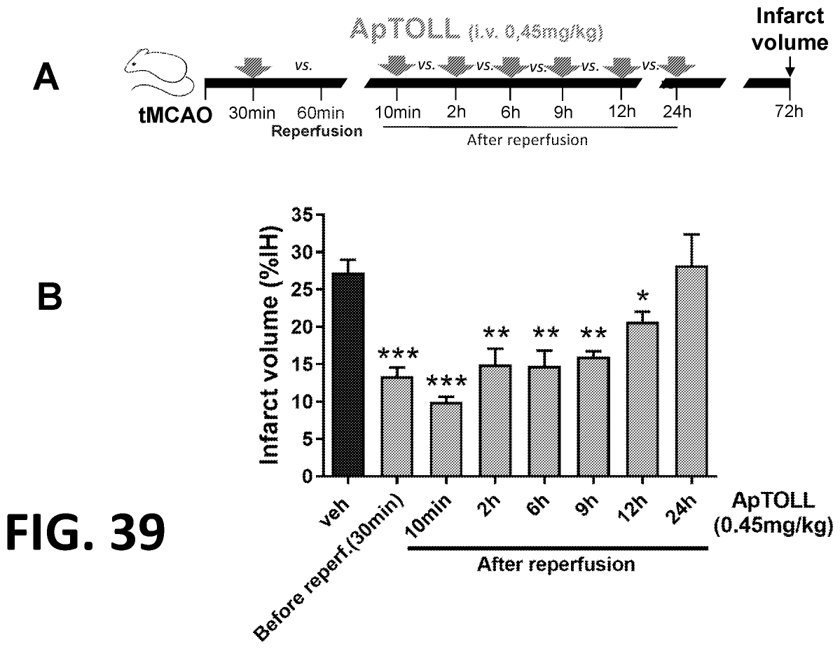

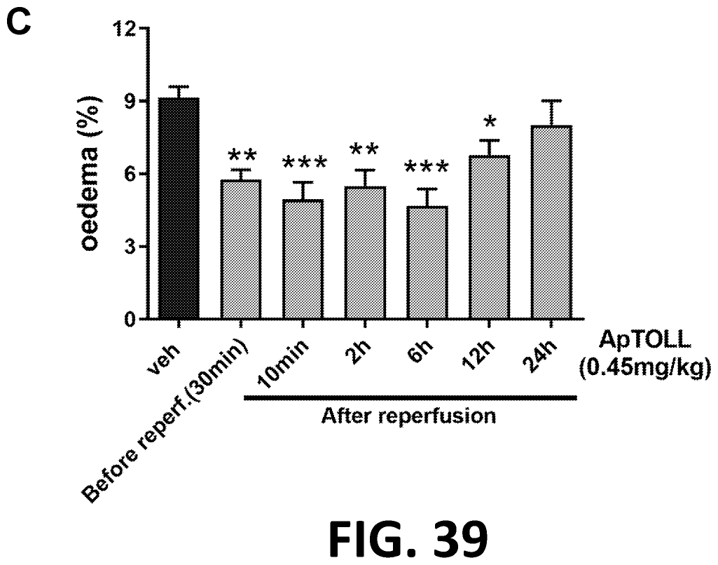

A second set of determinations were done in Wistar male rats (8-10 weeks old, 8 animals per group). Monofilament tMCAO model was performed and vehicle or ApTOLL (0.45 mg/kg) were injected 30 min before reperfusion (B.R.) or 10 min-2 h-6 h 9 h-12 h or 24 h after reperfusion. Infarct volume and edema was assessed 72 h after ischemia and the results confirm the protection mediated by ApTOLL up to 12 h after ischemia, the reduction in the infarct volume in this study was even higher than in those performed in pMCAO models, confirming a reduction of 50% (30 min B.R.), 65.5% (10 min), 45% (2, 6 h and 9 h) and 40% at 12 h. The effect was lost when the aptamer was administered 24 h after ischemia (FIG. 39). Interestingly, ApTOLL is also high protective when administered before reperfusion. These results indicate that the best moment to administer ApTOLL in combination with thrombectomy, based on infarct volume and edema reduction, is just before and some minutes after reperfusion. For this reason, and considering that the administration in humans is not a bolus but an infusion of 30 min, the infusion of ApTOLL in patients can start just before thrombectomy.

2.6. Characterization of Biomarkers after ApTOLL Administration

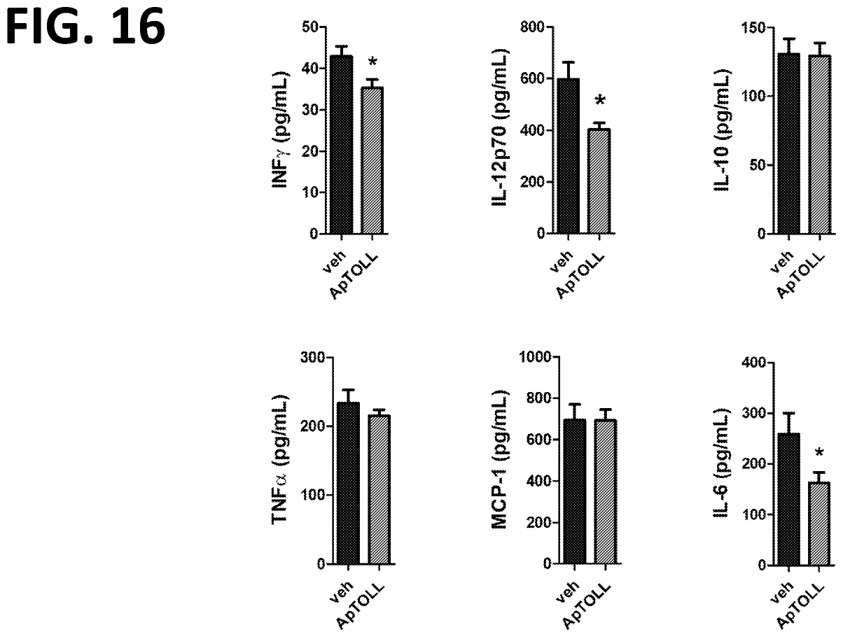

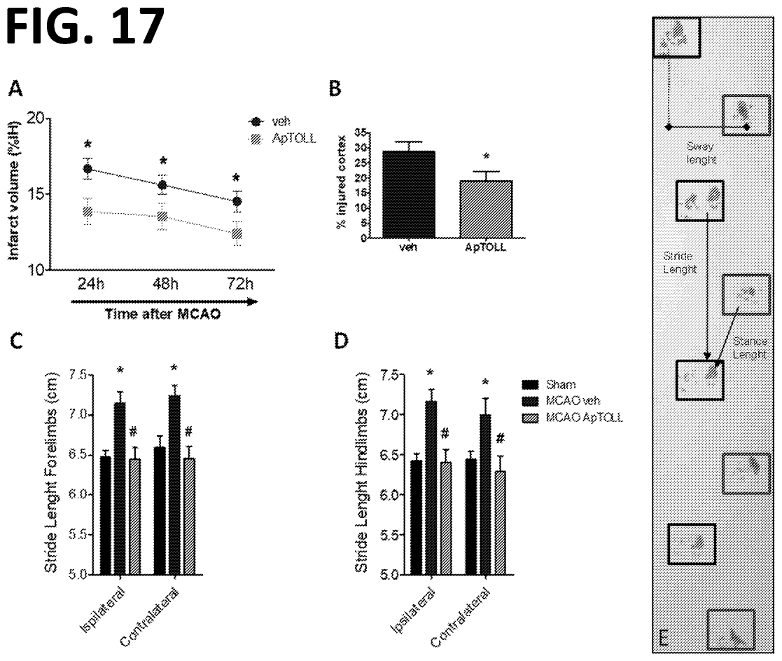

In order to identify possible biomarkers of stroke outcome in vivo, plasma samples from ischemic mice with 0.91 mg/kg ApTOLL/vehicle intraperitoneal treatment were obtained 24 h after pMCAO and analysed using CBA. To assess these biomarkers, a sub-study of the previous section pMCAO by electrocoagulation mouse model was performed and CBA technique was conducted. Briefly, The BD™ CBA Mouse Inflammation Kit is commonly used to quantitatively measure Interleukin-6 (IL-6), Interleukin-10 (IL-10), Monocyte Chemoattractant Protein-1 (MCP-1), Interferon-γ (IFN-γ), Tumor Necrosis Factor (TNF) and Interleukin-12p70 (IL-12p70) protein levels in a single sample. The results obtained in this study showed that treatment with ApTOLL significantly reduced the plasma levels of IL-6, IL-12p70 and IFN-γ, but not of TNF, IL-10 or MCP-1, at 24 hours after the ischemic insult, compared with the vehicle mice group (FIG. 16, n=8).

### 2.7. Long-Term Anatomical and Functional Relevance of the Protective Effect Induced by ApTOLL

In order to approximate the evaluation of the efficacy in vivo of ApTOLL to the end-point assessment of protection in stroke patients in clinical trials, we validated in an independent study the long-term permanence of infarct size reduction induced by ApTOLL when given acutely, as well as the correlation of the anatomical protection with functional, neurological performance in the mice. Protection achieved by 0.91 mg/kg of intravenous ApTOLL given 10 minutes after stroke was sustained throughout the sub-acute phase (up to 72 hours after stroke onset) (FIG. 17, panel A). Moreover, quantification of long-term injury size at 21 days after stroke indicated a preservation of the protective effect up to this time point when infarction is stabilized in this mouse model (C57Bl/6J male mice, 8-10 weeks old, subjected to permanent middle cerebral artery occlusion by ligature) (FIG. 17, panel B). The assessment of neurological function was performed by using the footprint test (FIG. 17, panel E). At 21 days after stroke, mice that received vehicle acutely showed an increase in the stride length as compared to sham-operated animals with no brain damage (FIG. 17 panel D), indicating altered limb control during the path. This effect was observed in both forelimbs and in the ipsilateral hind limb (FIG. 17, panel C; FIG. 17, panel D). Mice that received 0.91 mg/kg of ApTOLL 10 minutes after stroke showed absence of this neurological deficit at 21 days after stroke (FIG. 17, panel C; FIG. 17, panel D), indicating that reduction of infarct size induced by the aptamer related to improved functional performance in the long-term.

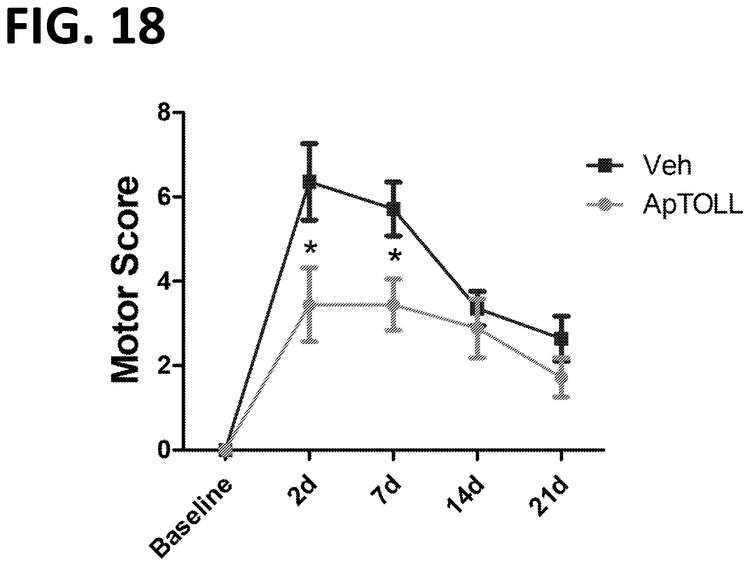

In another set of experiments, the neurological long-term outcome was evaluated in rats. Male Wistar rats were subjected to permanent cerebral ischemia by electrocoagulation model (n=8 animals per group). ApTOLL was administered 10 min after occlusion and motor evaluation was performed at 2, 7, 14 and 21 days thereafter. The results obtained showed a significant decrease in motor score at 2 and 7 days after stroke in rats treated with ApTOLL when compared with vehicle-treated rats (FIG. 18).

### 2.8. Antagonistic Efficacy Against TLR-4 Activation by LPS In Vivo in a Mouse Model of Sepsis

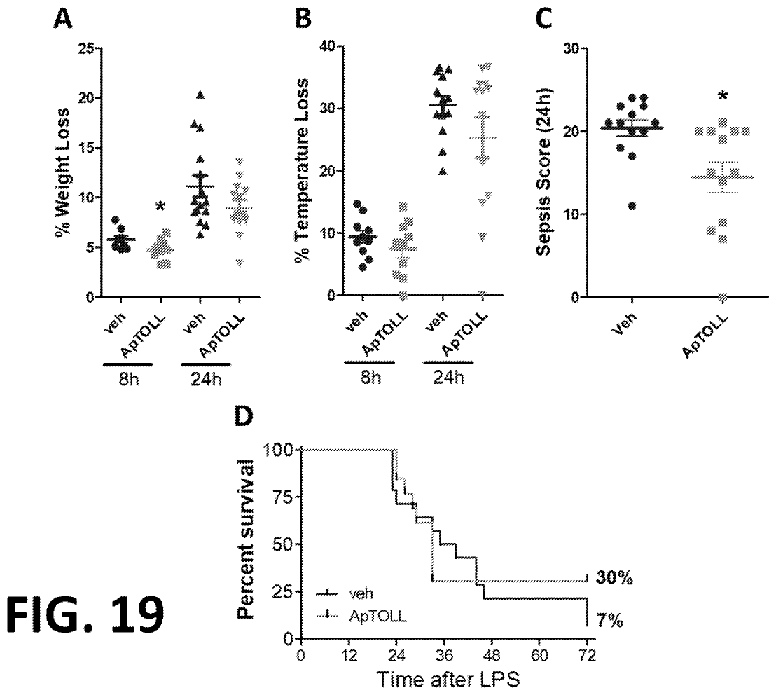

In order to validate the efficacy of ApTOLL as a TLR-4 antagonist in vivo, C57Bl/6J male mice, 8-10 weeks old, were injected intraperitoneally with 20 mg/kg ultrapure LPS. LPS injection led to endotoxemia in the mice, which was reflected in weight loss measurable at 8 hours and more severe at 24 hours (FIG. 19, panel A). Additionally, temperature loss was also noticeable at 8 hours and exacerbated at 24 hours (FIG. 19, panel B). A sepsis score was obtained by the quantification of multiple variables related to visible signs of endotoxemia (Schrum et al. (2014) BMC research notes 7:233) (FIG. 19, panel C). The group of animals injected with 0.91 mg/kg ApTOLL showed reduced % of weight loss at 8 hours as compared to animals injected with vehicle (FIG. 19, panel A), as well as a reduced sepsis score at 24 hours (FIG. 19, panel C). Survival of animals treated with aptamer ApTOLL was also higher at 72 hours after LPS injection (30% vs. 7% survival in mice treated with vehicle, FIG. 19, panel D), indicating that ApTOLL interfered with LPS activation of TLR-4, reducing the severity of induced endotoxemia.

### 2.9. Pharmacodynamic Drug Interactions

Studies to determine the interaction of ApTOLL with rt-PA were performed due to the fact that rt-PA is the only pharmacological treatment approved against ischemic stroke and, therefore, both drugs are likely to be used concomitantly in clinical practice.

Wistar naïve male rats (8-10 weeks old, 4 animals per group) were administered with ApTOLL, ApTOLL+rt-PA or rt-PA alone. Clinical signs were assessed after the administration and no sings appeared at any case.

### Example 3. ApTOLL Effects in Humans

### 3.1 A Double-Blind, Placebo-Controlled, Randomized, Phase Ia Clinical Study of ApTOLL for the Treatment of Acute Ischemic Stroke in Healthy Volunteers to Assess Tolerability and Pharmacokinetics

### Maximal Recommended Starting Dose in Humans

Calculation of the maximum recommended starting dose (MRSD) to be administered in healthy subjects:

### NOAEL (No Observed Adverse Effects Level):

Rats: no adverse effects observed with the higher dose, 50 mg/kg/day intravenously 14 days.

Cynomolgus Monkey: no adverse effects observed with the higher dose, 13.9 mg/kg/day (i.v. bolus) 14 days.

HED (human equivalent dose) was calculated from NOAEL considering conversion of animal doses to human equivalent based on body surface area. A correction factor of 10 was considered:

Rat: 50 mg/kg×0.162/10=0.81 mg/kg

Monkey: 13.9 mg/kg×0.324/10=0.45 mg/kg

Therefore, considering the lower calculated dose (0.45 mg/kg), the MRSD for a 70 kg weight person was 31.5 mg.

MABEL (minimum anticipated biological effect level):

Efficacy in rodent models of stroke:

a) pMCAO by ligature mouse model: protection for the 0.91 mg/kg dose (iv single bolus).

b) pMCAO by electrocoagulation mouse model: 0.91 mg/kg intraperitoneally.

c) tMCAO mouse model: 0.91 mg/kg intravenously.

d) pMCAO by electrocoagulation rat model: 0.45 mg/kg intravenously twice (10 min and 2 h after occlusion), similar efficacy to one dose.

f) tMCAO rat model: 0.45 mg/kg intravenously single bolus.

HED was calculated from MABEL considering conversion from animal doses to human equivalent based on body surface area. A correction factor of 10 was considered:

a) Mouse: 0.91 mg/kg×0.081/10=0.0073 mg/kg

b) Rat: 0.45 mg/kg×0.162/10=0.0073 mg/kg

Therefore, the MRSD for a 70 kg weight person was 0.5 mg.

We considered this dose because it was much lower than the MRSD calculated from NOAEL.

### Synopsis of the Study

Subjects: Healthy male or female without the possibility of becoming pregnant.

Design: single dose, intravenous administration (slow infusion), dose escalation with a maximum of 7 single dose levels, randomized, double-blind, placebo-controlled (saline solution), in healthy subjects, followed by multiple dose in healthy subjects.

The clinical trial had two parts:

### A—Single Dose Escalation in Healthy Subjects (Maximum 38 Subjects)

Dose Levels for Dose Escalation:
First dose-level: 2 subjects randomized to 0.7 mg or placebo (saline solution).
Second dose-level: 2 subjects randomized to 2.1 mg or placebo.
Third dose-level: 2 subjects randomized to 7 mg or placebo.
Fourth dose-level: 2 sentinel subjects randomized to 14 mg or placebo, followed by 6 subjects randomized to 14 mg (5 subjects) or placebo (1 subject).
Fifth dose level: 2 sentinel subjects randomized to 21 mg or placebo, followed by 5 subjects randomized to 21 mg (5 subjects) or placebo (1 subject).
Sixth dose level: 2 sentinel subjects randomized to 42 mg or placebo, followed by 6 subjects randomized to 42 mg (5 subjects) or placebo (1 subject).
Seventh dose level: 2 sentinel subjects randomized to 70 mg or placebo, followed by 6 subjects randomized to 70 mg (5 subjects) or placebo (1 subject).

Each subject was admitted to the Clinical Trials Unit since the night before dosing and until 2 days after the dose; if no safety problem was detected, he/she could go home 48 h after dosing and returned to the Clinical Trials Unit 72 h (3 days), 96 h (4 days), 120 h (5 days), 168 h (7 days), 240 h (10 days) and 336 h (14 days) after dosing.

There were at least two weeks separation between one dose level and the following one, allowing the Data Safety Monitoring Committee (DSMC) enough time to review all information and decide to continue with the next dose level. There were also one week between sentinel subjects and the other subjects of the same dose level.

### B—Multiple Doses in Healthy Subjects (8 Subjects)

2 sentinel subjects were randomized to three doses of drug (the higher safe dose of part A) or placebo at 0, 8 and 16 h.

Had no safety problems being reported, other 6 subjects were randomized to the same dose (5 subjects) or placebo (1 subject).

The volunteers left the Clinical Trials Unit 48 h after the drug administration, unless an adverse event was detected, in which case they remained admitted until their resolution.

### Drug Administration:

The drug (ApTOLL, Drug Substance-batch number 255887) was diluted in 100 mL saline and administered by slow intravenous infusion in 30 min by pump (considering a low infusion rate at the beginning of the infusion and increasing it thereafter and stopping it if some adverse event appears).

### Evaluation of the Subjects:

The safety of subjects was assessed through:

Record of all adverse events that occur during the study.

Physical examination.

Routine laboratory assessment (blood, biochemical and urine tests): screening, day 1 (predose), day 2, day 7 and day 14.

Toxics in urine: screening, day 1 (predose) and day 14.

Serology (HBV, HCV, HIV): screening.

Blood pressure, heart rate, respiratory rate and 12 lead ECG: baseline, at various times during admission and in each visit.
Pharmacokinetics: 15 blood samples (9 mL each) were taken at different times to define the pharmacokinetic profile of the drug: predose, 0.5 h, 1 h, 1.5 h, 2 h, 3 h, 4 h, 6 h, 8 h, 10 h, 12 h, 16 h, 20 h, 24 h and 32 h.
Pharmacodynamics: 4 blood samples (9 mL each) were taken at different times to evaluate cytokine levels induced by ex vivo lipopolysaccharide challenge: predose, 4 h, 8 h and 24 h.

### Results of the Phase Ia Clinical Trial

This was a Phase I, first-in-human, dose ascending, randomized, placebo-controlled clinical study to assess the tolerability and pharmacokinetics of ApTOLL in healthy volunteers. The main objectives of the study were the following:

(i) To evaluate the tolerability and pharmacokinetic characteristics of ApTOLL in healthy volunteers, after single dose administration in fasting conditions, following an ascending dosing scheme. The pharmacodynamic characteristics of this compound were also assessed; and,

(ii) To evaluate the tolerability and pharmacokinetic characteristics of ApTOLL in healthy volunteers, after multiple dose administration in fasting conditions. The pharmacodynamic characteristics of this compound were also assessed

The study was divided into 2 parts: the first one (Part A) was a dose escalation with a maximum of 7 single dose levels. Once this part was completed, a multiple dose (3 administrations) part (Part B) was carried out in healthy volunteers with the dose selected from the previous part. Both parts were randomized, double-blind, placebo-controlled (physiological saline solution). The study was conducted in healthy male subjects. The selected dose levels for dose escalation (Part A) and for the multiple dose (Part B) were the described above.

This clinical trial has been completed and conclusions are the following:

1. Regarding safety issues, no serious adverse events or significant analytic alterations were reported at any dose level.

2. Pharmacokinetics: fifteen blood samples (9 mL each) were taken at different times to define the pharmacokinetic profile of the drug: predose, 0.5, 1, 1.5, 2, 3, 4, 6, 8, 10, 12, 16, 20, 24, 32, 48 and 72 hours. In the SAD (Single Ascending Dose) part, Cmax data shows the maximum value at time 0.5 h after injection (at the end of the infusion) followed by an immediate decrease with time and with an estimated mean half-life was 8 h. ApTOLL levels are not quantifiable at time 72 h.

3. There were no clinically significant laboratory, vital signs or ECGs findings that were considered possibly related to the investigational drugs. Therefore, ApTOLL has a safety and tolerability profile similar to that of placebo.

### Example 4. ApTOLL Formulations

### 4.1. ApTOLL Formulation for Parenteral Administration in Humans

The IMP (Investigational Medicinal Product) is manufactured under full GMP conditions (FIG. 20). Briefly, the process of parenteral preparation should be done in a sterile area and develops as follows:

1. Preparation of Excipients solution:

Place approximately 80% of water for injections at temperature at 20-25° C. in the reactor provided with a stirrer. Add the Sodium chloride, the Potassium chloride, the Disodium phosphate dihydrate, the Potassium dihydrogen phosphate, the magnesium chloride hexahydrate, and stir until complete dissolution. Make the solution up to 100% volume with water for injections and check the pH of solution and adjust to 7.4 if necessary.

2. Filter the buffer solution through a 0.22 μm filter for sterilization.

3. Addition of active pharmaceutical ingredient Aptamer 4FT: place 90% volume of excipients solution in the jacketed glass reactor and dissolve the Aptamer 4FT in the buffer stirring until complete dissolution.

4. Adjustment of volume: make the solution up to the 100% volume with the excipients solution previously prepared. Stir for minimum 10 min.

5. For its biological activity it is necessary to dissolve it in PBS-1 mM Cl₂Mg in order to provide it a tertiary structure. After dissolution the aptamer must be heating up to 95° C.±2° C. Keep the solution at this temperature for minimum 10 minutes. Then, cool the solution up to 5° C.±3° C. Keep the solution at this temperature for 10 minutes.

6. Bioburden reduction:

Filter the solution through a 0.2 μm polyethersulfone sterile filter. Verify the integrity of the filter 1 (with water) with the minimum value of bubble point test.

Take a sample of 100 ml after Filter 1 for the Quality Control Department (Bioburden).

7. Sterilizing filtration through a 0.2 μm polyethersulfone filter, previously sterilized in a steam sterilizer.

8. Vials filling (under aseptic conditions): Vials washing and sterilization (vials: oven; stoppers: gamma irradiated, aluminum capsules: steam sterilizer). Filling and pre-closing process.

9. Lyophilization process: Freezing process; Drying (primary and secondary); Vials closing.

10. Capping and control of vials

### 4.2. ApTOLL Formulation for In Vivo Studies

The aptamer is freeze-dried and is kept at −20° C. until use. Avoid contamination:

wear gloves, filter tips, nuclease-free tubes. Dissolve in buffer A: PBS (phosphate buffered saline)+1 mM MgCl₂ free of nuclease.

1—Centrifuge the tubes with the lyophilized aptamer before adding the buffer.

2—Stock solution: add buffer A to the lyophilized aptamer and stir until completely dissolved. Divide into aliquots and keep at −20° C./−40° C. until use.

3—Working solution: dilute the stock solution in buffer A to the desired final concentration.

4—Folding process: heat the solution to 95° C. for 10 min and then keep on ice for 10 min.

5—Use the structured aptamer in the assay.

The structured aptamer maintains the functional conformation: 1 h at room temperature, 24 hours at 4° C.

### Example 5. Safety Pharmacology

### Effect on General Physiological Parameters

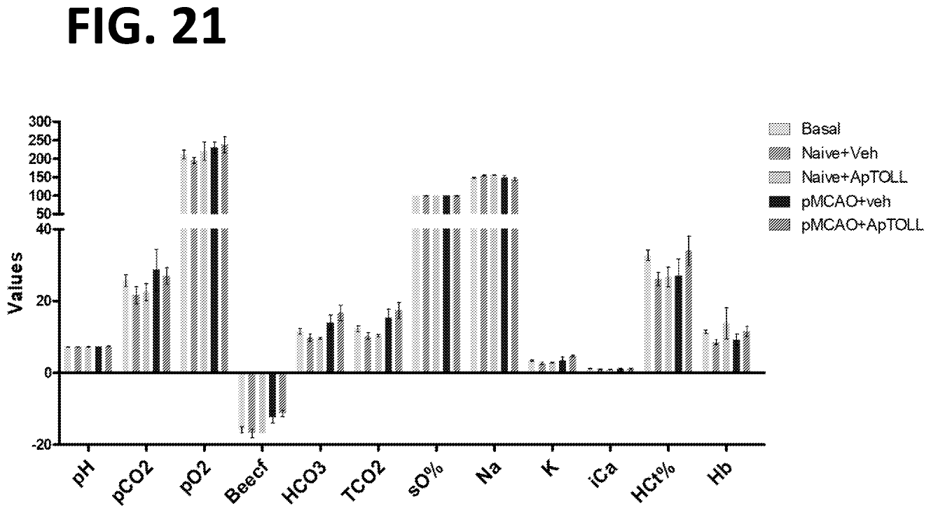

The potential effect of ApTOLL on general physiological parameters was tested in naïve and ischemic animals (C57Bl/6J male mice, 8-10 weeks old). Administration of ApTOLL showed no effect on any of the parameters measured when compared to vehicle administration (FIG. 21).

### Neurotoxicity

Different studies have shown that aptamers do not cross the blood-brain barrier (BBB) with few exceptions where highly specialized transport mechanisms are involved (Cheng et al. (2013) Mol Ther Nucleic Acids 2(1):e67). Specifically, in the case of ApTOLL, animal studies at a non-regulatory level have shown its distribution in different tissues such as lung or spleen within a few minutes of administration. However, the presence of ApTOLL in the brain has only been demonstrated after the induction of experimental stroke where the BBB is compromised, although in these animals the neuroprotective effect of the drug has been clearly demonstrated.

On the other hand, the short self-life of ApTOLL and its rapid degradation, as all oligonucleotides, will prevent it access to brain tissue in conditions where the BBB is not compromised (i.e. healthy volunteers). In fact, the research carried out to date on molecules of this nature by companies such as Ionis Pharmaceuticals (current name of the pioneer Isis Pharmaceuticals) or Opthotech, whose activity has also been centered in the CNS, is a clear example of the absence of neurotoxic events in its administration in humans. Clinical trials in which these companies have participated have directly injected intravitreally high concentrations of DNA and RNA molecules, not generating toxic effects in neuronal cells.

### In Vitro Neurotoxicity Evaluation

To evaluate the generalized toxicity of ApTOLL, a dose response curve was generated over eight concentrations from 0.01-30 μM in half-log increments. Each point was n=3. Mixed cultures of human iPSC-derived cortical glutamatergic (80%) and GABAergic (20%) neurons were cultured for one week before treatment with the test compound. Both positive (rotenone) and negative (DMSO) controls were included. Toxicity was evaluated at 72 hours following treatment using the CellTiter-Glo 2.0 Luminiscent Cell Viability Assay (Promega), which measured the total ATP concentration and is proportion to viable cell number.

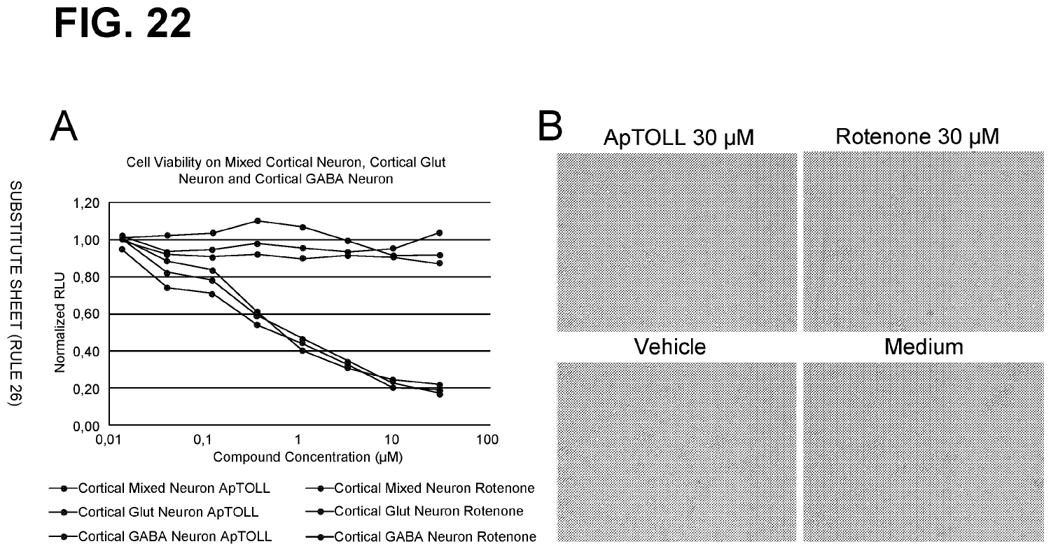

The results obtained in this study showed that, based on morphological criteria, all cultures displayed excellent cell health at the time of treatment. Moreover, the positive toxic compound rotenone, which inhibits the mitochondrial electron transport chain, showed a clear dose-dependent toxicity as assessed by measurement of cellular ATP levels. ApTOLL showed no toxicity up to the highest dose tested (30 μM). This lack of toxicity was observed in mixed cultures of glutamatergic (80%) and GABAergic (20%) neurons as well as in pure cultures of each of type (FIG. 22).

### Crossing Blood-Brain Barrier BBB

The objective was to determine whether the aptamer is able to cross the blood-brain barrier (BBB). To this end, Bend.3 cells (mice endothelial cells) and astrocytes (CTX-TNA2) located in the soil layer of a transwell insert, simulating the BBB. After incubation the presence of the aptamer in the medium was determined by qPCR. ApTOLL was tested in a concentration range of 40 nM to 4000 nM. A 76nt ssDNA aptamer able to pass through the BBB was used like positive control. The results obtained in this study showed that the aptamer is not able to cross the BBB in normal physiological conditions (TABLE 2), i.e., in the absence of any disease or condition altering the BBB permeability, e.g., a TLR-mediated disease or condition.

TABLE 2
Results obtained in the Gaiker 3722 study

| | Length | Recovered aptamer | |
|---|---|---|---|
| Sample | (nucleotides) | (% total) | SEM (%) |
| R6BBB-4F | 76 | 62.3965 | 1.846 |
| R6BBB-11R | 76 | 70.8828 | 4.686 |
| R6BBB-15R | 76 | 68.8169 | 0.144 |
| R6BBB-4R | 76 | 44.7621 | 3.888 |
| ApTOLL (40 nM) | 59 | 0.0021 | 0.001 |
| ApTOLL (400 nM) | 59 | 0.0045 | 0.001 |
| ApTOLL (4000 nM) | 59 | 0.0056 | 0.002 |

### Neurotoxicity

Neurotoxicity evaluation in rats: This study was performed as a part of the Principal Toxicity study: 2-week Toxicity Study in Rats Followed by a 1-week Recovery Period. The purpose of the toxicity study was to assess the toxicity effects of ApTOLL when administered intravenously to rats at 5, 25 and 50 mg/kg/day once daily for a period of 2 weeks. The study was performed in compliance with GLP. Observations (sensory reactivity, grip strength and locomotor activity) were made in recovery animals at pre-treatment and in week 2 of treatment. The results obtained in this study showed that there were no relevant changes between groups in the FOB records.

Neurotoxicity evaluation in ischemic rats: After ischemia, the permeability of the BBB is compromised and ApTOLL is able to reach the brain tissue. Because of this, potential neurotoxicity derived from ApTOLL administration was evaluated by using the modified Irwin test. This test consisted of a set of assays to evaluate the presence of neurotoxic effects derived from drugs. To this end, ischemic rats (n=8) injected with ApTOLL (0.45 mg/kg) or vehicle, were tested. The experimental model used in this study was the permanent ischemia by electrocoagulation. No differences were detected after administration of ApTOLL either in treated nor vehicle-animals. Therefore, no effects were observed on parameters related to muscular tone, coordination and sensorimotor responses. No alterations in the open-field test were reported.

### Effects in Respiratory Function

The purpose of this study was to evaluate the possible side-effects of ApTOLL on respiratory rate, tidal volume and minute volume in the rat.

ApTOLL was administered by intravenous bolus injection to male Sprague-Dawley rats (8/group) at doses of 5, 25 and 50 mg/kg, in order to assess effects on respiratory rate, tidal volume and minute volume. Two additional groups received either an equivalent volume (3 mL/kg) of vehicle as a single intravenous bolus dose, or an oral dose (10 mL/kg) of baclofen at a dose of 20 mg/kg (positive control). Respiratory rate, tidal volume and minute volume were reported at 0 (pre-dose), 30, 60, 90, 120, 150, 180, 210 and 240 min post-dose.

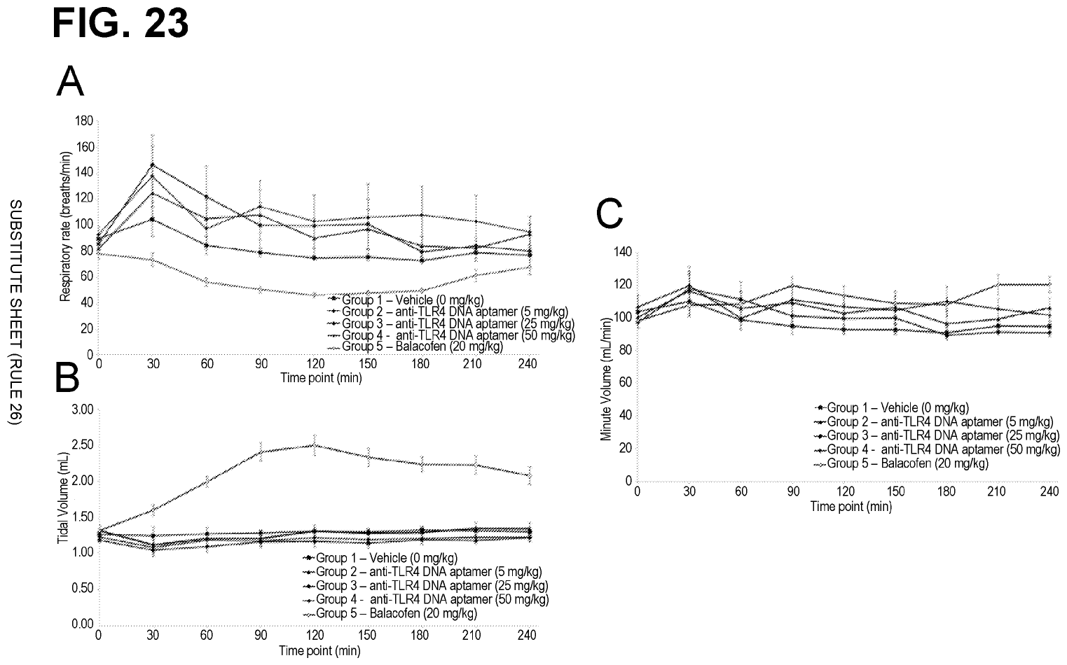

ApTOLL, administered by intravenous bolus at doses of 5, 25 and 50 mg/kg produced no biologically relevant effects on respiratory rate, tidal volume or minute volume that were considered to be test item related (FIG. 23). Baclofen administered orally at a dose of 20 mg/kg produced significantly lower respiratory rates and a significantly higher tidal volume values. There were no adverse clinical signs observed.

### Effects in Cardiovascular System

This study was in compliance with GLP standards as a part of the Principal Toxicity study: 2-week Toxicity Study in Rats Followed by a 1-week Recovery Period. The purpose of this study was to assess the effects of ApTOLL in cardiovascular system when administered twice daily six hours apart by intravenous route (bolus) to Cynomolgus monkey for a period of 14 days.

ApTOLL was administered in a total of 32 monkeys (0.7-2.3-6.9 mg/kg/b.i.d). The heart rate, P-wave duration and amplitude, P-Q interval, QRS interval and Q-T interval were measured using a representative section of the electrocardiogram from lead II. Correction of the QT interval for heart rate was also calculated. Recordings were made at pretest, on treatment day 13 (after first daily dose) and during recovery. Results showed that there were no findings related to treatment with the test item.

### Example 6. Pharmacokinetics and Product Metabolism in Animals

### Distribution

### Binding to Plasmatic Proteins

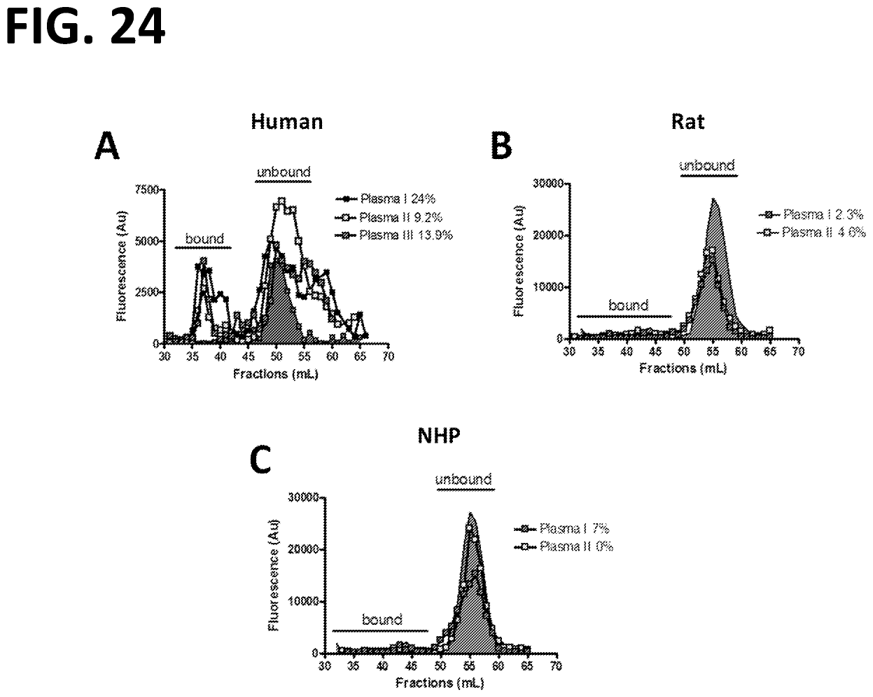

The fraction of ApTOLL binding plasmatic proteins was determined. For this purpose, ApTOLL conjugated to Alexa-488 was used. The percentage of aptamer bound to plasma proteins was calculated by the ratio (sum of fluorescence in all fractions/total fluorescence) ×100 in two-three different human, rat and NHP samples (FIG. 24).

In all cases, a solution of aptamer in absence of plasmatic protein was run as a control for the determination of the unbound elution peak. Fraction of ApTOLL bound to plasmatic proteins was 15.7% for human samples, and 3.5% in rat and NHP plasma.

### In Vivo Distribution of ApTOLL in the Target Organ (Brain)

The distribution of ApTOLL, peripheral and central studies using Alexa Fluor 488-labelled ApTOLL was performed.

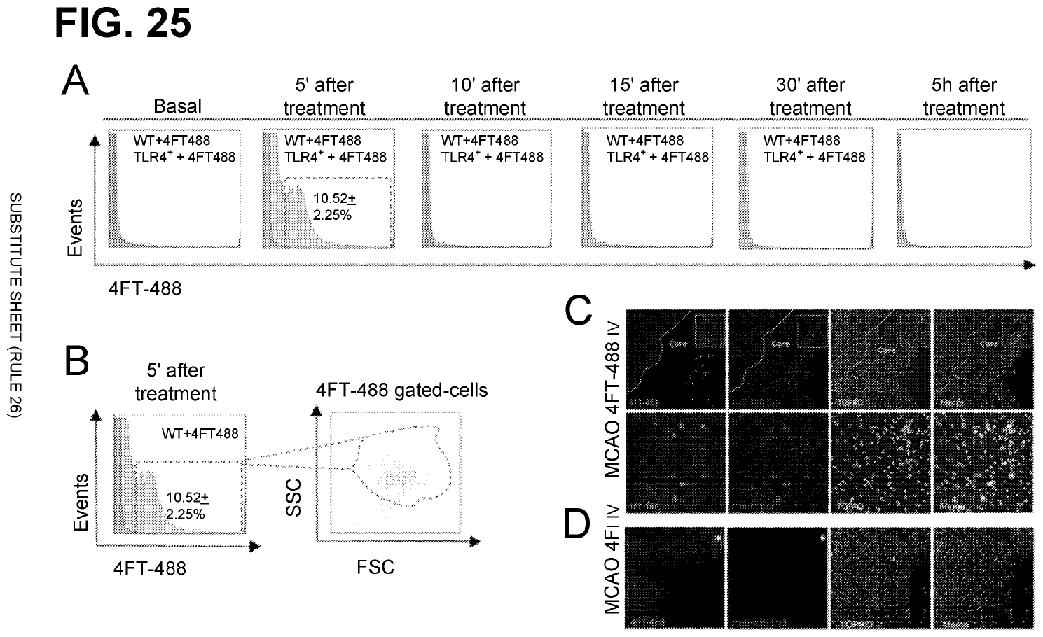

First, a flow cytometric analysis of aptamer in blood from TLR4+/+ and TLR4−/− mice (n=3-5) subjected to pMCAO that received an intravenous administration of Alexa Fluor 488-labelled ApTOLL (0.9 mg/kg) 10 min after the surgery was performed. Basal samples and serial blood samples from tail were obtained at 5, 10, 15, 30 min and 5 h after aptamer administration. The results demonstrated that ApTOLL was detected in blood 5 min after the administration in pMCAO TLR4+/+ mice (FIG. 25, panel A). However, in TLR4−/− mice, it was not detected aptamer binding at any of the times studied. Also, Alexa Fluor 488-labelled ApTOLL gated cells were mainly in the granulocyte region based on forward scatter (FSC) and side scatter (SSC) gating strategy (FIG. 25, panel B).

Moreover, ApTOLL conjugated with Alexa-488 was used to detect its presence in the brain after intravenous injection. Six mice (C57Bl/6J male mice, 8-10 weeks old) were subjected to pMCAO in order to reproduce the conditions of the blood-brain barrier in the ischemic brain, injected with the labelled aptamer 10 minutes after pMCAO and brains were collected and processed for immunofluorescence at 24 hours. Green fluorescence was observed in the ischemic region (FIG. 25, panel C), and specificity of the signal was confirmed by incubation of the brain sections with a Cy3-conjugated anti-Alexa488 antibody (FIG. 25, panel C, red). Fluorescence was absent in animals injected with non-labelled aptamer (FIG. 25, panel D). These observations indicated that ApTOLL was present within the infarcted brain tissue at least 24 hours after intravenous injection.

### Metabolism

### In Vitro Stability in Human, Rat and Monkey Plasma and Against Nucleases

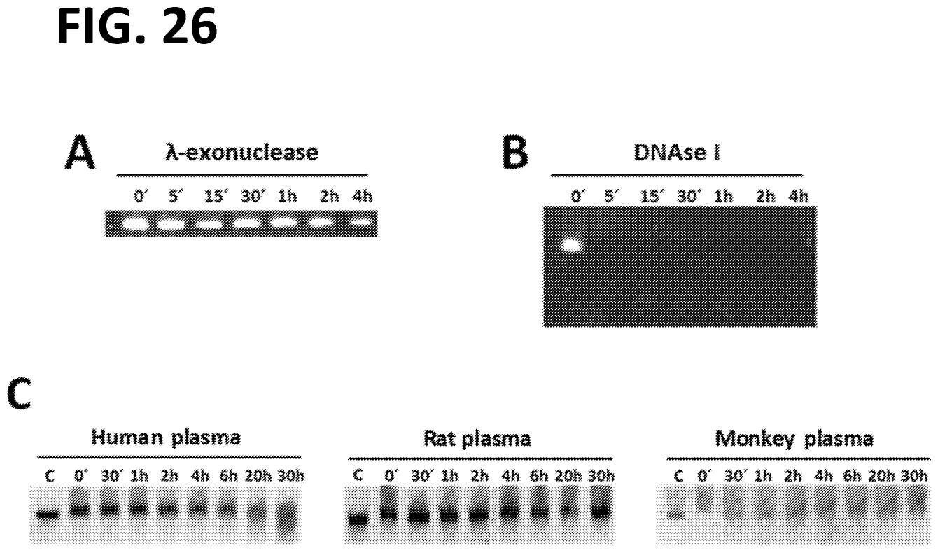

The integrity of ApTOLL in the presence of λ-exonuclease and DNAseI treatments, as well as, the stability in rat, monkey or human plasma were quantified. The results showed that ApTOLL was resistant to λ-exonuclease even after 4 h of incubation (FIG. 26, panel A). This result was in agreement with the lack of a 3″-end phosphate in the synthetic aptamer required for the λ-exonuclease activity. In contrast, degradation of ApTOLL was patent after 5 min exposure to DNAse I (FIG. 26, panel B). When ApTOLL was incubated in rat, monkey and human plasma in physiological conditions, a time-dependent degradation was observed (FIG. 26, panel C). Keeping into account that the indication proposed for ApTOLL is acute stroke treatment, this half-life profile was considered optimal for a short-term, acute exposure of TLR-4 to the aptamer, e.g., to treat an acute TLR-4 mediated disease or conditions such as ischemic stroke.

### Pharmacokinetic Drug Interactions

The purpose of this study was to test ApTOLL in binding, enzyme and uptake, in vitro absorption and in vitro metabolism assays. ApTOLL was tested at 20 nM due to the results obtained in Pharmacodynamic studies (please refer to previous sections).

### In Vitro Pharmacology: Uptake Assays and Binding Assays

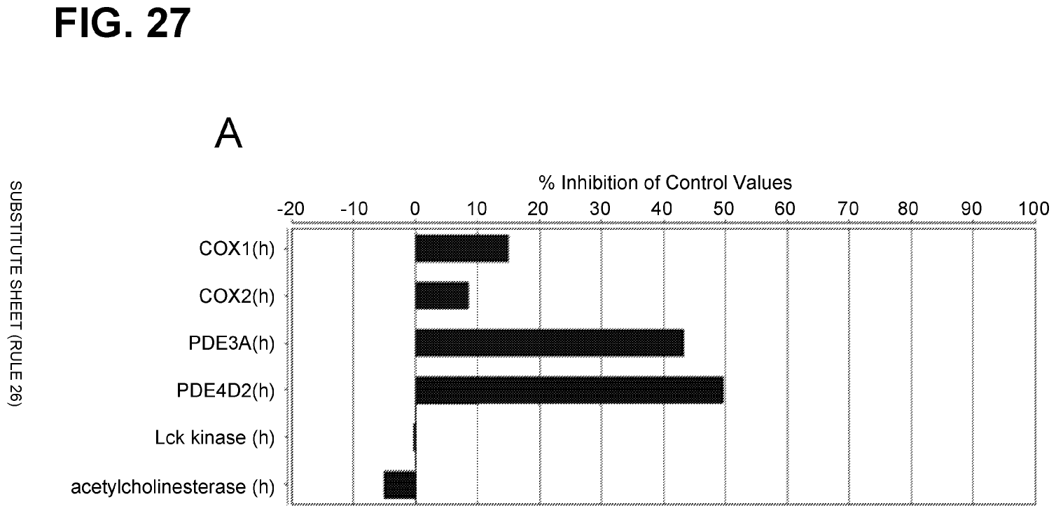

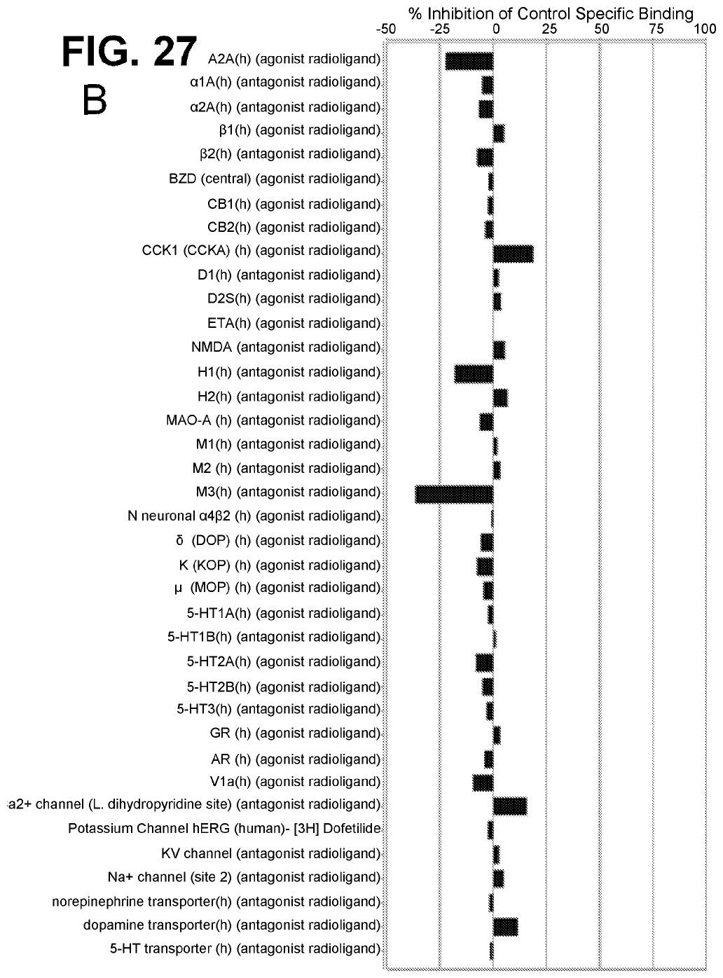

SafetyScreen44™ panel was performed to enable early identification of significant off-target interactions with ApTOLL. All 44 targets (GPCRs, Ion Channels, Kinases, Nuclear Receptors, Transporters and other Non-Kinase Enzymes) were selected to bring together both robustness (each assay is HTS-compatible) and the strategic choice of information-rich targets. Compound binding was calculated as a % inhibition of the binding of a radioactively labeled ligand specific for each target. Compound enzyme inhibition effect was calculated as a % inhibition of control enzyme activity. Results showing an inhibition higher than 50% were considered to represent significant effects of the test compounds. Such effects were not observed at any of the targets studied (FIG. 27). In each experiment and if applicable, the respective reference compound was tested concurrently with ApTOLL, and the data were compared with historical values.

Despite the values obtained in enzyme assays did not show significant effects, a deeper characterization was performed for PDE3A and PDE4D2 and no significant results were obtained. Therefore, no inhibitory effect was detected in any target selected.

### ADME-Tox: In Vitro Absorption

These assays were designed to assess how a compound may affect major drug transporters. Specifically, these assays tested the potential inhibition of drug transporters that may interfere with absorption, distribution or excretion of ApTOLL.

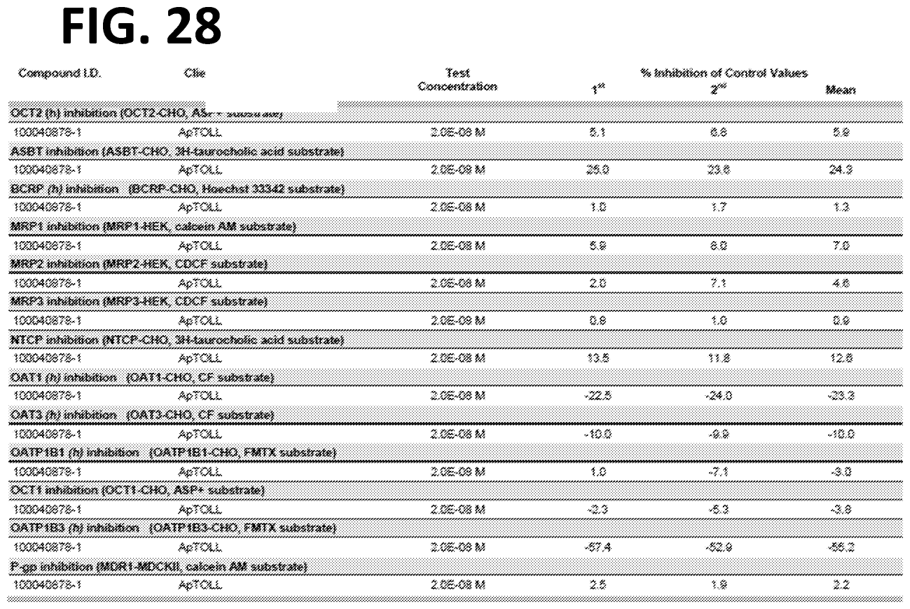

ApTOLL transporter inhibition effect was calculated as a % inhibition of vehicle control activity. Results showing an inhibition higher than 50% were considered to represent significant effects of the test compounds. Such effects were not observed at any of the receptors studied (FIG. 28).

Despite the values obtained in drug transporters assays did not show significant effects, a deep characterization was performed for ASBT and no effects were detected in this study conditions.

### Cytochrome Inhibition Assay

These assays were performed to assess how ApTOLL may affect major drug metabolizing enzymes characterizing a potential inhibitory effect of the Cytochrome P450 (CYP) enzyme(s) that could lead to a buildup of a co-administered compound.

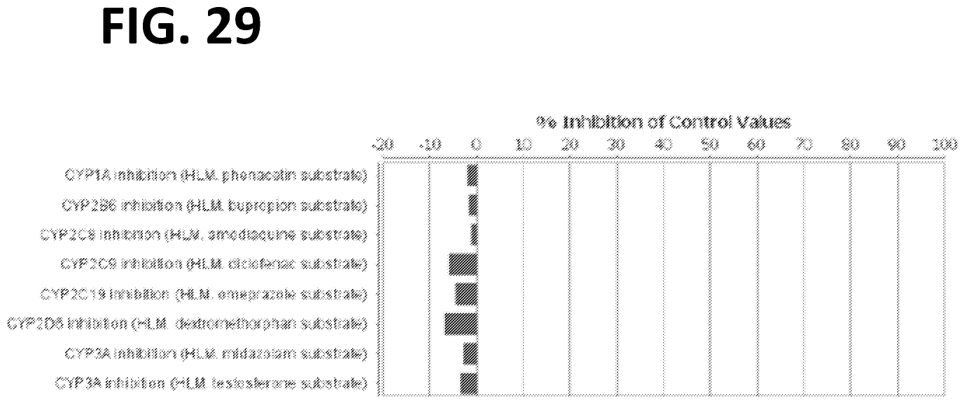

ApTOLL CYP enzyme inhibition effect was calculated as a % inhibition of vehicle control activity. Results showing an inhibition higher than 50% were considered to represent significant effects of the test compounds. Such effects were not observed at any of the enzymes studied (FIG. 29).

Moreover, in these assays, induction of CYP enzymes was evaluated to prevent a decreased plasma concentration of ApTOLL or co-administered compounds. ApTOLL was administered at different concentrations (2-20-200 nM) to make better phenomenon characterization.

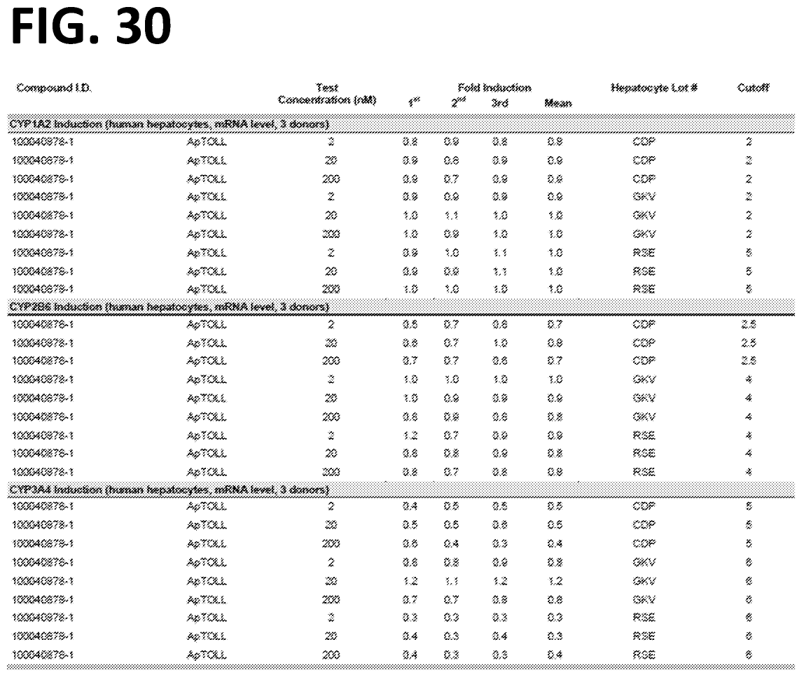

ApTOLL CYP enzymes induction effect was calculated as fold induction of vehicle control activity. Results showing a stimulation higher than 50% are considered to represent significant effects of the test compounds. Such effects were not observed at any of the enzymes studied (FIG. 30).

### Other Pharmacokinetic Studies

### Intravenous Pharmacokinetic Study in Sprague Dawley Rats

The aim of this study was to obtain the pharmacokinetic profile of ApTOLL after single intravenous bolus administration at 0.45, 1 and 2 mg/kg to female Sprague Dawley rats. Therefore, nine female rats (10-12 weeks-old) were administered ApTOLL at 0.45, 1 and 2 mg/kg at 1 mL/kg by single intravenous bolus in the lateral tail vein.

Blood samples were obtained the day of administration from the lateral tail vein at the following times: 1 min (immediately after administration), 5, 15, 30 min and 1, 2, 4, 8 and 24 hours.

Blood samples (approximately 250 μL each) were collected into EDTA-K3 tubes and plasma was prepared. The tubes were placed in a cold bath for no more than 30 minutes until they were centrifuged at 1900 g for 10 minutes at 2-8° C. Following centrifugation, at least 100 μL of plasma was transferred into a plastic (polypropylene) tube and stored at −80±10° C. until shipment.

The pharmacokinetic analysis showed that ApTOLL concentrations in plasma were quantifiable in all animals at all dose levels (where samples available) up to 8 hours postdose. In general, the maximum plasma concentration was observed (Tmax) at the first timepoint (1 minute). Mean Cmax and AUCt values are summarized below:

TABLE 3
Mean Cmax and AUCt values (with standard deviations in parentheses)
after single administration of ApTOLL in female Sprague Dawley rats

| Dose level (mg/kg) | Cₘₐₓ (ng/mL) | AUCₜ (ng · h/mL) |
|---|---|---|
| 0.45 | 8100 (3470) | 2310 (510) |
| 1 | 18400 (6000) | 3530 (260) |
| 2 | 39700 (10500) | 6710 (660) |

The main conclusions drawn from this study were:
Animals were administered at the correct dose levels.
Blood samples were collected from all animals except for animal no. 3F at 8 h due to experimental difficulties. Samples were obtained at the correct times with the exception of five samples which deviated by 1 and 5 min at the sampling time-points of 2 h, 4 h and 24 h.
There were no clinical signs.
The study samples analyzed according to the qualified method showed good assay performance.
Plasma ApTOLL concentrations were quantifiable in all animals at all dose levels (where samples available) up to 8 hours postdose and Tmax was at the first timepoint (1 minute post-dose).
Cmax showed a linear kinetics over the dose range 0.45 to 2 mg/kg whereas exposure (AUCt) presented a non-linear kinetics over the same dose range.

### Pharmacodynamics and Pharmacokinetics Conclusions

The main conclusions drawn of PD and PK studies are summarized below:
ApTOLL has been selected from a wide number of aptamers designed due to its appropriated antagonistic profile against TLR-4.
In vitro, ApTOLL exhibits good pharmacodynamic profile at 20 and 200 nM.
Ka of ApTOLL in monkey and human monocyte cells is 30-60 nM.
ApTOLL does not show any interaction with other TLRs (neither agonistic nor antagonistic).
In mice and rats, ApTOLL induces protection after cerebral ischemia both in the short and long term and in different experimental models.
The therapeutic window of ApTOLL is, at least, 6 h after stroke.
ApTOLL does not exhibit neither effects on physiological parameters nor neurotoxicity.
No clinical signs in PK study have been reported. Study samples analyzed according to the qualified method showed good assay performance.
Plasma ApTOLL concentrations were quantifiable in all animals at all dose levels (where samples available) up to 8 hours post-dose and Tmax was at the first timepoint (1 minute post-dose).
Cₘₐₓ showed a linear kinetics over the dose range 0.45 to 2 mg/kg whereas exposure (AUCt) presented a non-linear kinetics over the same dose range.

### Example 7. Toxicology

### Single Dose Toxicity

### In Vitro Toxicology: Effect of ApTOLL on Cell Viability

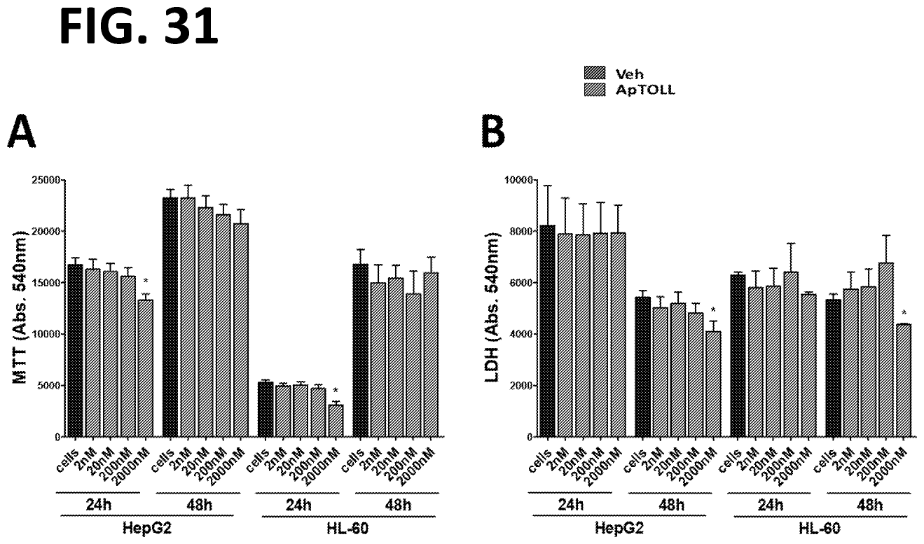

Potential cell toxicity of ApTOLL was assessed by incubation with two different cell lines routinely used for these studies (Hep-G2 and HL60). Cell viability was quantified by the MTT and LDH assays at 24 and 48 hours after addition of aptamers (2-2000 nM) to the incubation medium (FIG. 31). Only concentrations 100-fold higher than the biologically active concentrations showed a modest effect on cell viability at 24 hours (FIG. 31, panel A). Moreover, a decrease in LDH levels after 48 h of incubation at higher doses potentially related to a decreased cell number in these cultures (FIG. 31, panel B).

### Repeat-Dose Toxicity

For Repeat-Dose Toxicity studies, rat was selected as rodent model (due to similar pharmacology) and monkey as a non-rodent model (due to its human-TLR-4-homology).

### Preliminary Toxicity Study in Sprague Dawley Rats

The purpose of this study was to assess the toxicity effects of ApTOLL following intravenous administration to rats daily for seven consecutive days. The study indicated potential target organs and provided a rational basis for the selection of dose levels for a subsequent two-week toxicity study.

ApTOLL was administered intravenously once daily to Sprague Dawley rats for 7 days. The animals were allocated to four treatment groups as follows:

TABLE 4
Design of the different groups involved in the PW28XN study

| Groups | 1* | 2 | 3 | 4 |
|---|---|---|---|---|
| Test Item | — | ApTOLL | ApTOLL | ApTOLL |
| Dose Levels | 0 mg/kg/day | 5 mg/kg/day | 25 mg/kg/day | 50 mg/kg/day |
| Dose | 0 mg/mL | 5 mg/mL | 25 mg/mL | 50 mg/mL |
| Concentration | | | | |
| Males | 1-5 | 6-10 | 11-15 | 16-20 |
| Females | 101-105 | 106-110 | 111-115 | 116-120 |

All animals were observed throughout the study twice daily for viability/mortality. Daily cage-side observations were performed during acclimatization and pre-test, and clinical signs were also recorded daily during the treatment period; the injection site was inspected for local signs before and after dosing. Food consumption was recorded before treatment starts and twice weekly during treatment period. Body weight was recorded once during pre-test, twice a week during the treatment period and before sacrifice (unfasted). Blood samples for hematology and clinical biochemistry were collected at the end of the treatment period (Day 8) from all animals. All main animals were sacrificed and necropsied following completion of treatment and organs were weighed. A full set of tissues was retained but they were not examined.

The results of the study are summarized as follows:
All animals survived until the end of the treatment period and were sacrificed as scheduled on day 8.
No clinical signs related to the test item were recorded in animals administered at 5, 25 and 50 mg/kg/day ApTOLL. However, from day 5 to day 7, vocalization and discomfort on the tail was observed in some animals including control.
No local signs were observed at the injection site at any administered dose.
At 5, 25 and 50 mg/kg/day ApTOLL, there were no relevant differences in food consumption taking into account the sample size and the magnitude of the changes. No noticeable changes in food consumption were observed in the Control group.
No noticeable differences in body weights were observed at 5, 25 or 50 mg/kg/day ApTOLL.
There were no toxicological relevant hematology or biochemistry effects.
The administration of ApTOLL did not cause test-item-related macroscopic findings at necropsy.
There were no statistically significant differences in the mean absolute or adjusted weight of the collected organs: brain, heart, kidneys, liver, lungs and bronchi and spleen.
There was no observed cytokine response to ApTOLL across the treatment groups or between the time points except for a slight increase in IL-6 in the females for both pre-treatment and terminal groups.

Under the conditions of this study, ApTOLL administered intravenously once daily to Sprague Dawley rats for 7 days up to 50 mg/kg/day did not cause any toxicological effects.

### 2-Week Toxicity Study in Rats Followed by a 1-Week Recovery Period

The purpose of this toxicity study was to assess the toxicity effects of ApTOLL when administered intravenously to rats at 5, 25 and 50 mg/kg/day once daily for a period of 2 weeks. In addition, the reversibility or progression of any treatment-related changes or delayed toxicity was assessed in several animals after a 1-week treatment-free recovery period. The study indicated potential target organs and provided a rational basis for risk assessment in humans.

A total of 74 males and 74 females, 5-7 weeks old, SD rats were used in the study. Rats were distributed in 4 groups with 15-20 males and 15-20 females each. ApTOLL was administered intravenously, single bolus, once daily. The duration of treatment was:
14 days (Main and Recovery)
14 days (Toxicokinetic)
1 day (Biomarkers)

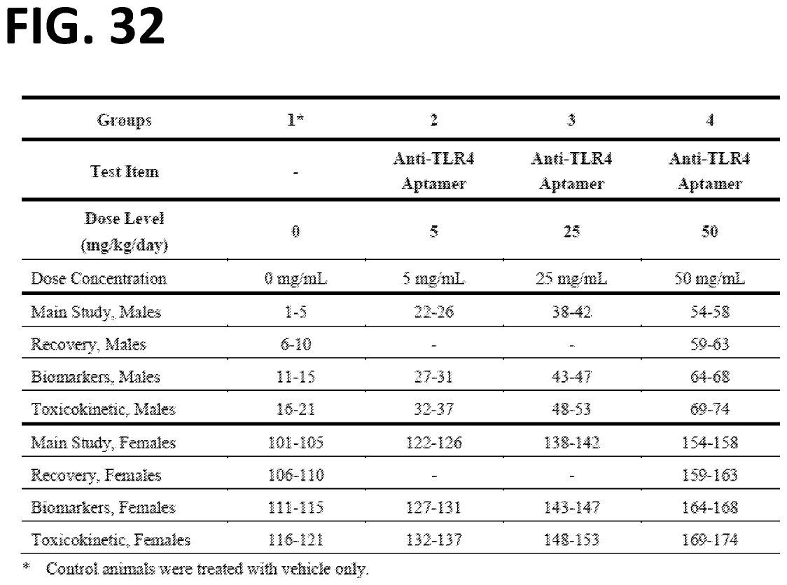

Treatment groups and doses are shown in FIG. 32.

The results obtained in this study showed that there were no clinical signs after ApTOLL administration. Moreover, rats after treatment with ApTOLL did not show any alteration in performing the FOB test. Hematology, coagulation, clinical biochemistry and urinalysis were within normal parameters. Macropathology was normal as well. No statistically significant dose-related differences were observed in cytokine levels after administration of ApTOLL with the exception of IP-10. The amount of significant differences in IP-10 levels, compared to the control group, increased with an increase in the dose for all day 1 samples. Small but significant increases were observed in male rats from groups 3 and 4 at day 14; however, there were no significant increases observed in rats at day 14 when compared to the control group.

As no significant toxicological findings (i.e. adverse alteration in morphology, functional capacity, growth, development or span in the treated animals) were observed after 14 days of continuous and daily administration of ApTOLL to SD rats (50 mg/kg), the principal conclusion of the abovementioned study was that this compound did not induce remarkable toxicological alterations.

Therefore, 50 mg/kg ApTOLL was considered as the NOAEL (No Observed Adverse Effects Level). 50 mg/kg was the maximum dose used. The use of higher dose could lead to higher NOAEL values.

Toxicokinetic Evaluation: Quantifiable concentrations were found at 5 minutes post-dose in 3 males and 2 females on day 1 (range 1.0-18.3 ng/mL) and in 3 males and 1 female on Day 14 (1.4-11.4 ng/mL). These concentrations were much lower than those observed in the treated animals at the equivalent times; however, the source of this apparent discrepancy was not identified.

Inter individual variation in plasma of ApTOLL concentrations was high, with coefficients of variation generally being greater than 70%, and in the range 2.2% to 173%.

Overall, the time at which the maximum plasma concentration was observed (Tmax) was at the first timepoint (5 minutes), as expected following intravenous administration. Nonetheless, the plasma concentration-time profile of the females receiving the 25 mg/kg/day dose level on day 1 suggests that these animals did not receive the dose intravenously (e.g., the dose was administered intraarterially or intramuscularly).

Maximum mean plasma concentrations (Cmax) of ApTOLL and the areas under the mean plasma ApTOLL concentration time curves up to the time of the last quantifiable plasma concentration (AUCt) on day 1 and day 14 are summarized below:

TABLE 5
Summary of the Cₘₐₓ and AUCₜ values in Sprague Dawley rat model

| | Cₘₐₓ (ng/mL) | | | | AUCₜ (ng · h/mL) | | | |
|---|---|---|---|---|---|---|---|---|
| Dose level | Day 1 | | Day 14 | | Day 1 | | Day 14 | |
| (mg/kg/day) | Males | Females | Males | Females | Males | Females | Males | Females |
| 5 | 51300 | 33900 | 63500 | 47700 | 18800 | 13000 | 22200 | 16700 |
| 25 | 98300 | 5060 | 93800 | 140000 | 44100 | 3010 | 34000 | 49300 |
| 50 | 327000 | 357000 | 198000 | 147000 | 118000 | 128000 | 70300 | 57000 |

The extent of systemic exposure of rats to ApTOLL appeared to be characterized by nonlinear (dose dependent) kinetics over the dose range 5 to 50 mg/kg/day on day 1 and day 14.

Overall, ApTOLL concentration was generally similar to that of males and no accumulation after 14-day repeated intravenous bolus administration was observed.

### Preliminary 7-Day Intravenous (Bolus) Toxicity Study in the Cynomolgus Monkey

The purpose of this study was to assess the toxicity effects of ApTOLL following intravenous administration to monkeys daily for seven consecutive days. The study indicated potential target organs and provided a rational basis for the selection of dose levels for a subsequent two-week toxicity study.

ApTOLL was administered intravenously once daily to 6 Cynomolgus Monkeys (3 males and 3 females, 24-36 months old) for 7 days. The animals were allocated to four treatment groups as follows:

TABLE 6
Design of the groups involved in the Cynomolgus Monkey model study

| | Test item | | |
|---|---|---|---|
| Dose Level | Group 1 | Group 2 | Group 3 |
| mg/kg/day | 0.7 | 2.3 | 6.9 |
| Males | 1 | 2 | 3 |
| Females | 4 | 5 | 6 |

The results obtained in this study show that there were no relevant clinical signs observed after ApTOLL administration. Food consumption and body weights remained at normal parameters. No treatment- or sex-related effect could be determined for any of the cytokines determined (IFN-γ, IL-1β, IL-2, IL-4, IL-6 and TNF-α) or for neither of the complement activation complexes (CH50 and C5B-9) determined. Neither macroscopic findings nor variation in organ weights were reported.

Based on these results, the MTD (Maximum Tolerated Dose) after a daily administration for one week was established at 6.9 mg/kg/day ApTOLL.

Toxicokinetics: Maximum plasma concentrations (Cmax) of Anti-TLR-4 DNA Aptamer and areas under the plasma concentration-time curve up to the time of the last quantifiable plasma concentration (AUCt) are summarized below:

TABLE 7
Summarization of the Cₘₐₓ and AUCₜ values in the
Cynomolgus Monkey model study

| Dose level | Cₘₐₓ (ng/mL) | | AUCₜ(ng · h/mL) | |
|---|---|---|---|---|
| mg/kg/day | Males | Females | Males | Females |
| 0.7 | 15800 | 12200 | 2550 | 2100 |
| 2.3 | 30900 | 34600 | 5040 | 5620 |
| 6.9 | 164000 | 142000 | 27400 | 26800 |

Therefore, the Cₘₐₓ values and extent of systemic exposure of monkeys to ApTOLL, after a single dose, appeared to be characterized by linear (dose-independent) kinetics over the dose range 0.7 to 6.9 mg/kg/day.

The terminal half-life (t½) was in the range 0.8 to 1.4 hours, and appeared to be independent of dose and sex. The total plasma clearance (Cl) was in the range 252 to 409 mL/h/kg and the mean volume of distribution at steady-state (Vss) was in the range 34.0 to 68.3 mL/kg.

### A 14-Day Repeated Dose Toxicity Study in the Cynomolgus Monkey by Intravenous Route Followed by a 1-Week Recovery Period

The purpose of this study was to assess the cumulative toxicity and toxicokinetics of ApTOLL when administered twice daily six hours apart by intravenous route (bolus) to Cynomolgus monkey for a period of 14 days. The reversibility or progression of treatment-related changes or any delayed toxicity was assessed during a 1-week recovery period for some animals following the treatment period.

A total of 32 animals (16 males and 16 females, 28-29 months old) were allocated in four groups which differed in the concentration of ApTOLL administrated to the animal:

TABLE 8
Design of the groups involved in the Cynomolgus Monkey model study

| Dose Level | Group 1 | Group 2 | Group 3 | Group 4 |
|---|---|---|---|---|
| mg/kg/day | 0 | 1.4 | 4.6 | 13.9 |
| No. of males | 3 (main) | 3 (main) | 3 (main) | 3 (main) |
| | 2 (recovery) | | | 2 (recovery) |
| No. of females | 3 (main) | 3 (main) | 3 (main) | 3 (main) |
| | 2 (recovery) | | | 2 (recovery) |

The results obtained in this study showed that there are no toxicological signs related with ApTOLL administration. Only perivascular fibrosis and subcutaneous fibrosis at the injection sites, including the control group were observed at the end of treatment with a partial recovery one week after. No relevant clinical signs were observed: all animals survived until the end of the study, no effects in food consumption nor body weights changes, no findings in ophthalmoscopy. No treatment- or sex-related effect could be observed for any of the cytokines determined (IFN-γ, IL-1β, IL-2, IL-4, IL-6 and TNF-α) or for neither of the complement activation complexes (CH50 and C5B-9) determined. No macroscopic or microscopic changes were reported.

In conclusion, the dose of 13.9 mg/kg/day ApTOLL was considered the NOAEL when ApTOLL was administered twice daily (6 hours apart) by intravenous (bolus) route to Cynomolgus monkey for a period of 14 days.

### Toxicokinetics

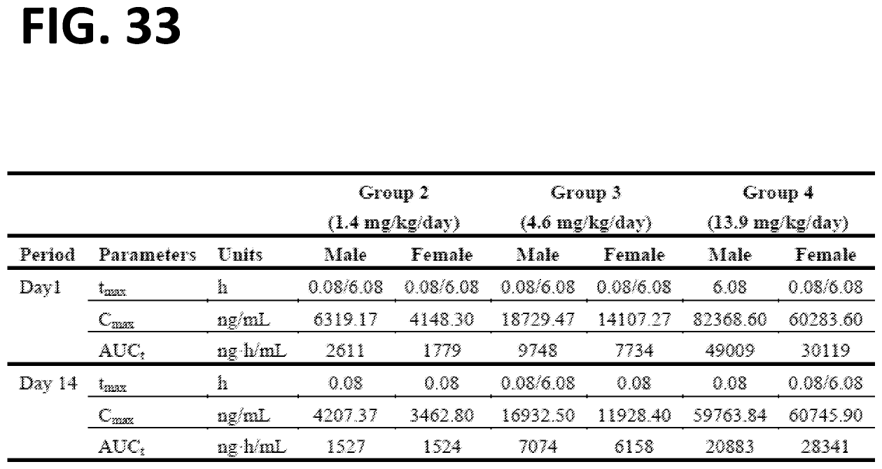

A summary of the main toxicokinetic parameters of 4FT Aptamer is given in the FIG. 33.

No accumulation of ApTOLL was observed under these dosage regimens.

Comparable exposure was observed between males and females in all groups. The male/female ratios ranged from 1.0 to 1.5 for Cmax and from 0.7 to 1.6 for AUCt.

### Genetoxicity

### In Vitro

The genotoxicity assays were designed according to ICH S2(R2) guidelines. A test battery including Ames and in vitro micronucleus assays (with and without metabolic activation by S9) was performed. The Ames fluctuation test assessed the mutagenic potential of compounds and the in vitro micronucleus assay complemented the Ames fluctuation test in the evaluation of genotoxicity effects such as chromosomal damage. Cytotoxicity was assessed in parallel during each assay to identify possible false negatives due to cytotoxicity.

To evaluate the various types of genotoxicity several in vitro assays were used as a screening tool. In each experiment and, if applicable, the respective reference compound was tested concurrently with ApTOLL, and the data were compared with historical values.

Bacterial cytotoxicity: Bacterial cytotoxicity of a compound was tested in parallel with the Ames assay to identify possible false negatives due to cytotoxicity. The Cell Number % Cytotoxicity was an index based on cell numbers, in which:

$$Cytotoxicity ( \% ) = 100 - \frac{Number of treated cells * 100}{Number of control cells}$$

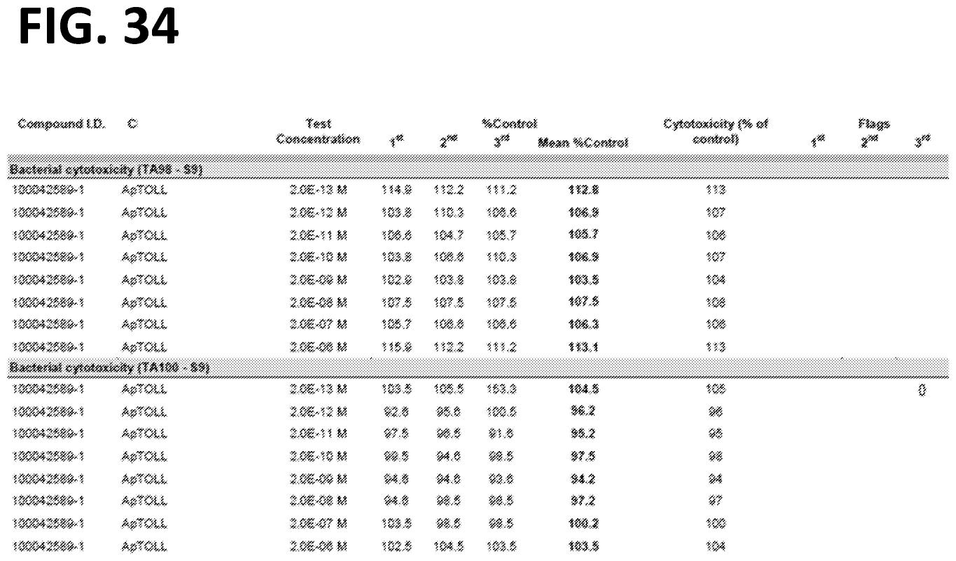

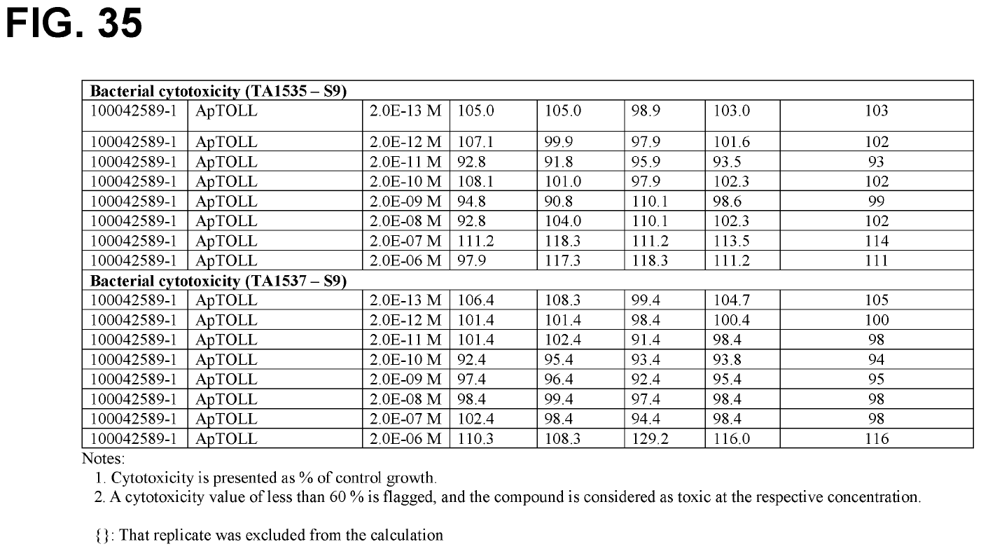

Compounds with a growth of 60% or lower than control were flagged and considered cytotoxic. Under these conditions, results obtained in this study show a non-bacterial cytotoxic effect (FIG. 34 and FIG. 35).

Ames test: Ames test was performed to determine if ApTOLL could cause direct DNA mutation. Gene mutations can easily be measured in bacteria, caused by a change in the growth requirements. The Ames test was conducted using Salmonella typhimurium, a widely used bacterial assay for the identification of compounds that can produce gene mutations, showing a high predictive value with rodent carcinogenicity tests. The Ames test typically uses five strains of Salmonella with preexisting mutations that render the bacteria unable to synthesize the essential amino-acid histidine, and, as a result, cannot grow in histidine-free medium.

The Ames fluctuation assay was performed in 384-well plates using four Salmonella strains, TA98, TA100, TA1535 and TA1537. The bacterial plates were incubated with the test compounds for 96 hours and bacterial growth was measured spectrophotometrically using a pH indicator that changes color in response to the acidification of the media due to bacterial growth. Metabolic activation was achieved by using rat liver S9 fraction. To prevent false negatives due to bactericidal or bacteriostatic effects, a bacterial cytotoxicity assay was conducted in parallel with the Ames fluctuation assay.

Wells that displayed bacteria growth due to the reversion of the histidine mutation (as judged by the ratio of OD₄₃₀/OD₅₇₀ being greater than 1.0) were counted and recorded as positive counts. The significance of the positive counts between the treatment (in the presence of test compound) and the control (in the absence of test compound) were calculated using the one-tailed Fisher's exact test.

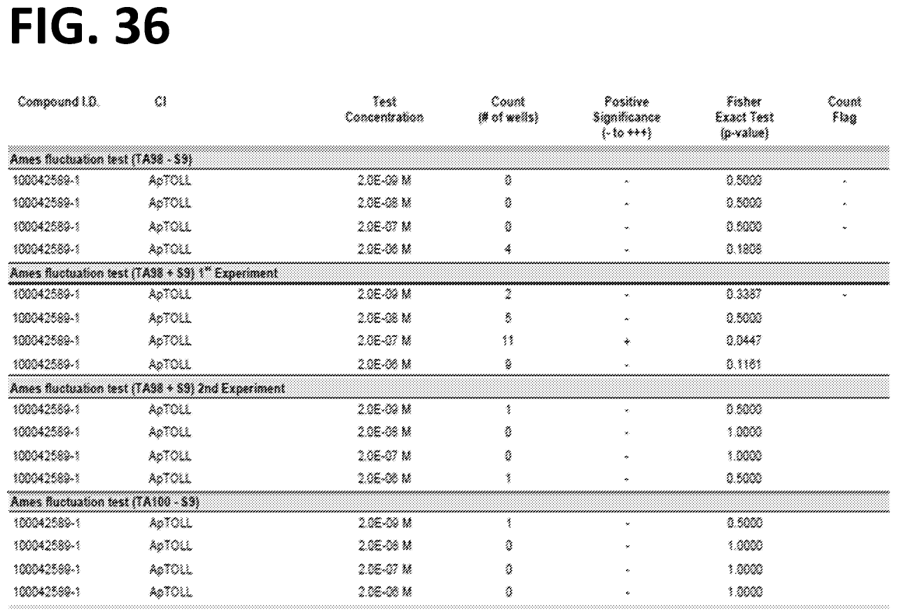

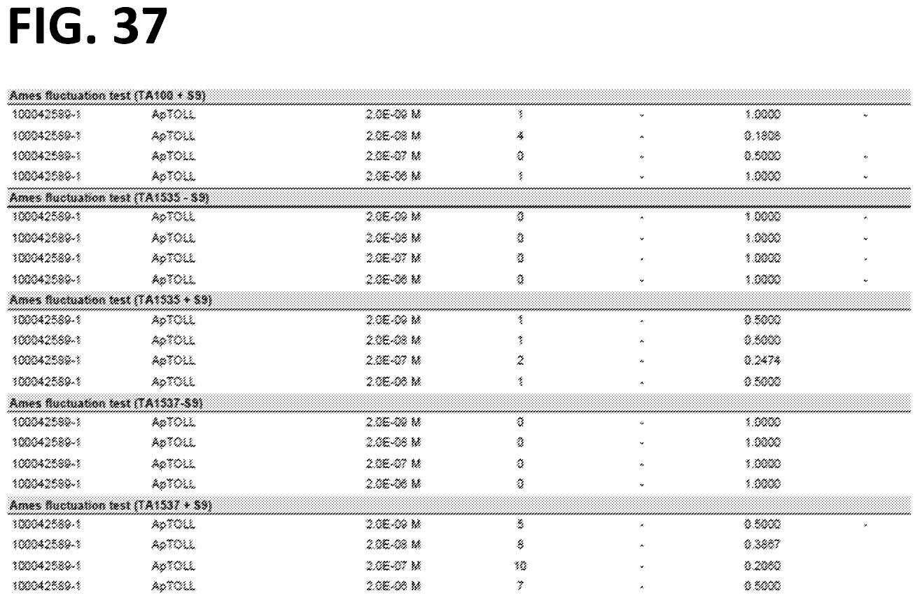

The results obtained in this study did not show any significant effect in the Ames test, therefore, no direct DNA mutations after ApTOLL administration were identified (FIG. 36 and FIG. 37).

In vitro Micronucleus assay: This assay was performed to assess whether ApTOLL could cause chromosomal damage by introducing double stranded DNA breaks or impacting mitotic cell division. Micronucleus formation is a hallmark of genotoxicity, and the micronucleus assay is an important component of genotoxicity screening. Micronuclei are chromatin-containing bodies that represent fragments or even whole chromosomes that were not incorporated into a daughter cell nucleus at mitosis. The purpose of the assay was to detect those agents that induce chromosome damage leading to the induction of micronuclei in interphase cells.

The in vitro micronucleus assay was conducted in CHO-K1 cells. The cells were seeded in 96-well plates and treated with the test compounds for 24 h (without S9) and for 4 h (with S9). Cytochalasin B, which is a cytokinesis blocker, was added after 24 h and the cells were incubated for an additional 24 h, after which the cells were fixed and scored for micronuclei. The percent of micronucleated cells was calculated. Cytokinesis Block Proliferation Index (CBPI) % Cytotoxicity uses a modified version of the (CBPI). This method takes advantage of the fact that cytotoxicity very often induces cell cycle arrest, which is reflected in a decreased ratio of bi-nucleated to mononucleated cells when using cytochalasin B. A CBPI of 1 is equivalent to 100% cytotoxicity.

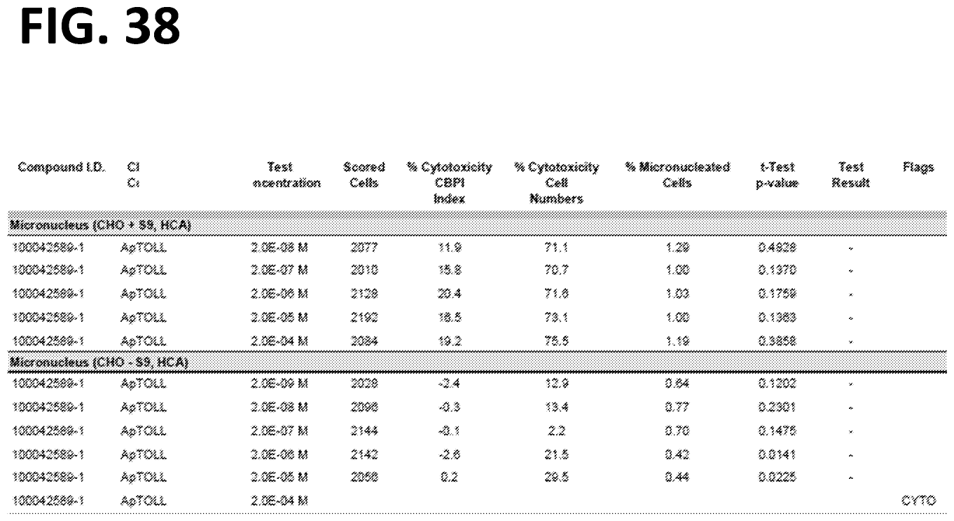

The results obtained in this assay did not show any induction of micronuclei (FIG. 38).

### Local Tolerance

### Preliminary Toxicity Study in Sprague Dawley Rats

No toxicity-related signs at the injection site were detected at any studied concentration of ApTOLL.

### 2-Week Toxicity Study in Rats Followed by a 1-Week Recovery Period

Overall, there were no local signs at the injection site. However, from day 5 to 7, two males (group 4) presented erythema on the tail.

Preliminary 7-day Intravenous (Bolus) Toxicity Study in the Cynomolgus Monkey

Bruises at the injection sites were recorded. No other local alterations were observed.

### A 14-Day Repeated Dose Toxicity Study in the Cynomolgus Monkey by Intravenous Route Followed by a 1-Week Recovery Period

Dark areas seen at some of the venous injection sites were registered at macroscopic examination including the control group. Microscopic evaluation resulted in treatment-related findings at the four administration sites (perivascular fibrosis and subcutaneous fibrosis). The vehicle was considered the main elicitor of the fibrosis, which was contributed to by the twice daily injection procedure. The assessment of animals following the 1-week recovery period demonstrated partial recovery at the four administration sites.

### Example 8. Efficacy of ApTOLL in Left Ventricular Remodeling after Myocardial Infarction

To evaluate the impact of ApTOLL in the left ventricular remodeling after myocardial infarction, a model of myocardial ischemia-reperfusion injury through ligation of the left anterior descending artery (LAD) was conducted in rats. Briefly, the ligation of the LAD coronary artery to perform the ischemia was kept for 30 minutes. Then, the ligation was removed to allow the reperfusion. One single dose of ApTOLL (0.45 mg/kg) was injected intravenously, in a single bolus given 10 minutes after reperfusion. A solution of PBS with 1 mM MgCl2 was used as a control (vehicle). The echocardiographic parameters ejection fraction and fractional shortening were recorded in basal conditions and 72 h post-infarction.

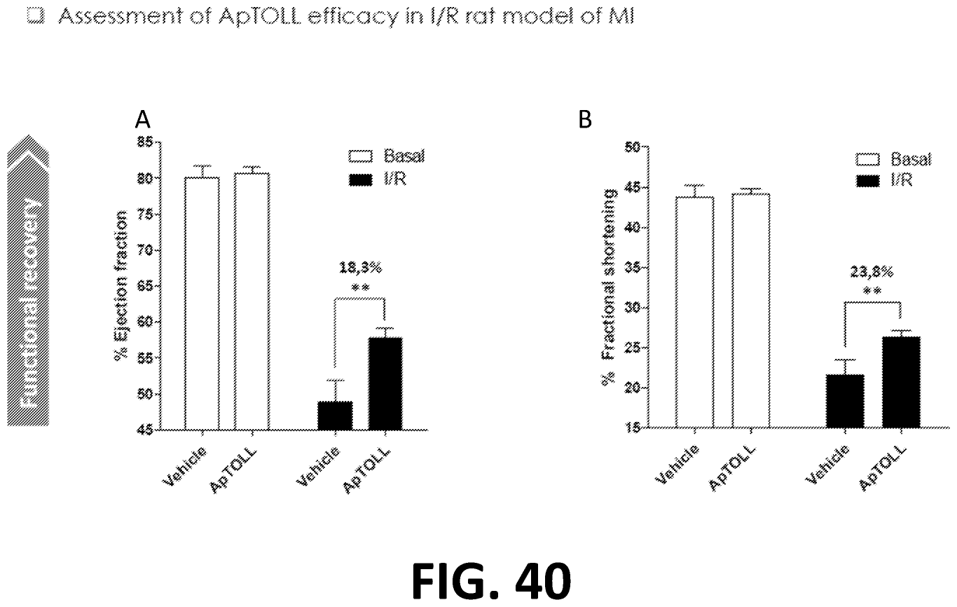

Results: As depicted in the graphs, the ejection fraction and the fractional shortening were dramatically affected after the ischemia-reperfusion injury. However, the administration of one single dose of ApTOLL (0.45 mg/Kg) showed 18.3% and 23.8% recovery respectively (FIG. 40). All these data together shown ApTOLL has a protective effect of the heart's muscular contractility after myocardial infarction and this effect might preserve heart functionality.

### Example 9. Efficacy of ApTOLL in an Experimental Autoimmune Encephalomyelitis (EAE) Mice Model

9.1. Assay with ApTOLL at 0.91 mg/kg

To evaluate the potential neuroprotective effect of ApTOLL in the inflammatory component of multiple sclerosis (MS), we have conducted assays in the EAE mice model. This model is the most commonly used experimental model for the human inflammatory demyelinating disease, MS.

Anesthetized female C57/BL6 mice of 7-week-old were immunized subcutaneously with MOG35-55 peptide in incomplete Freund Adjuvant containing Mycobacterium tuberculosis. Then, mice were intravenously administered pertussis toxin by injection in the tail vein at the time of immunization and 48 h later. One single dose of ApTOLL (0.91 mg/Kg) was intravenously administrated in the tail, in a single bolus given at the first sign of the EAE (onset). A solution of PBS with 1 mM MgCl₂ was used as a control (vehicle). Mice were examined daily for neurological symptoms, since the onset and during all the assay time, using the following clinical score criteria: 0, no detectable signs; 0.25, tip tail dropped; 0.5, half tail dropped; 0.75, tail dropped except the base; 1, flabby tail; 1.5, flabby tail with partial hind-limb weakness; 2, evident hind-limb weakness; 2.5, unilateral partial hind-limb paralysis; 3, complete bilateral hind-limb paralysis; 3.5, total hind-limb paralysis with partial forelimb weakness; 4, complete bilateral forelimb paralysis; 5, death.

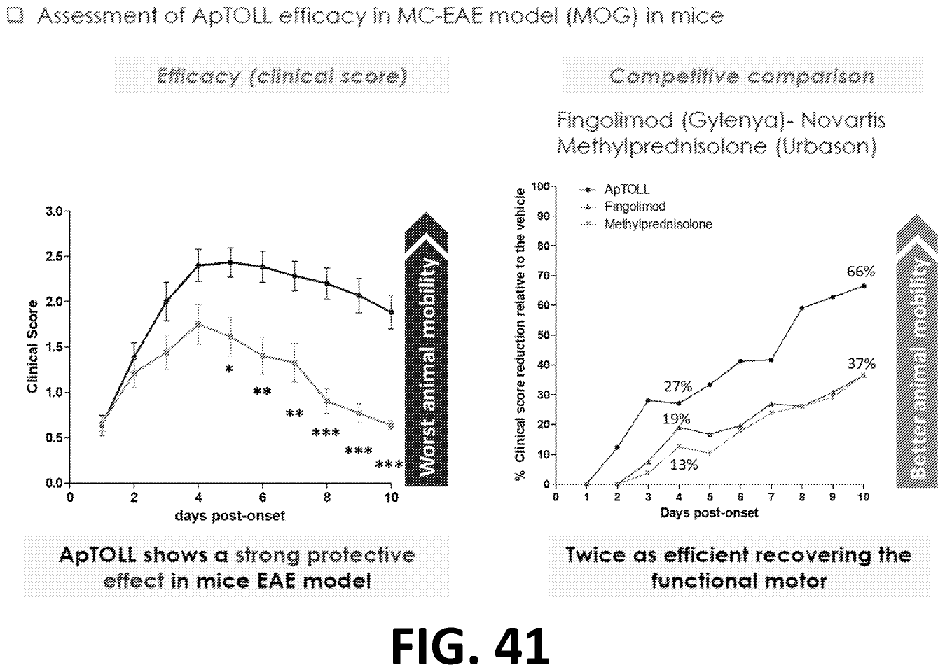

Results: The administration of ApTOLL showed a 27% reduction of the clinical score at the peak of the symptoms (4 days post-onset) and even higher reduction (66%) at the end of the assay if compare with the vehicle (FIG. 41). This data indicated a neuroprotective effect of ApTOLL with impact in the inflammatory component of the disease.

9.2. Efficacy of ApTOLL Administered 24 h after Disease Onset

An independent assay was conducted, administrating one single dose of ApTOLL (0.91 mg/Kg) intravenously, in a single bolus given, 24 h later to the first sign of the EAE (onset).

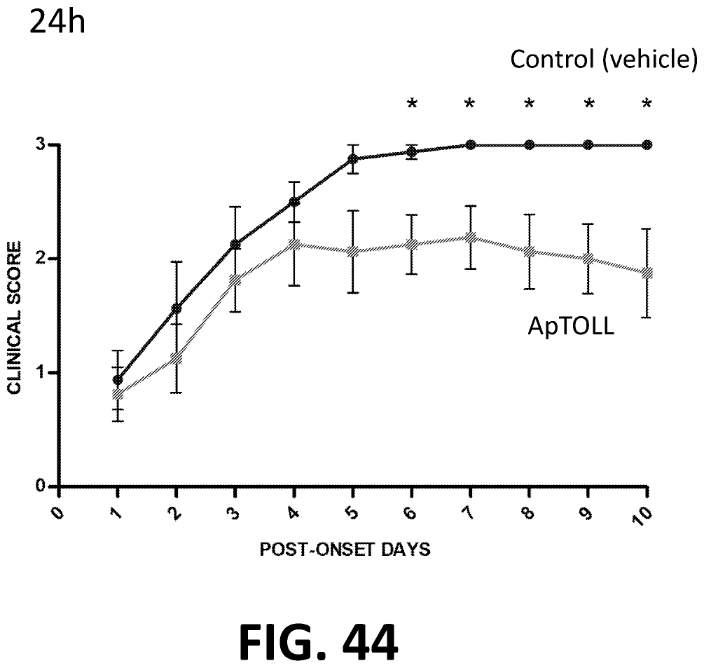

Results: A clear reduction in the clinical score was observed when ApTOLL was injected 24 hours after the onset (FIG. 44).

9.3. Assay with ApTOLL at Different Doses: 0.45, 0.91, 1.82, 3.6 mg/kg

The assay was conducted as in section 9.1.

Tissue processing: The extraction and processing of nerve tissue was carried out 10 days post-onset (time of drug administration). A euthanasic dose of DOLETHAL®, transcardiac perfusion was performed, by which, using a peristaltic pump, the tissue was fixed with 4% paraformaldehyde (PFA) through the circulatory system. The tissue was then obtained, in this case, brain and spinal cord. Next, the tissue was washed several times for 10 min in PB, cryoprotected by three successive passes in growing concentrations of sucrose in PB, and finally frozen in OCT, separated into different parts (brain, cerebellum, cervical spinal cord, thoracic spinal cord in two parts (T1 and T2) and lumbar spinal cord). T1 was used for this study and cut with a cryostat at a thickness of 20 micrometers in a transverse plane.

Eriochrome cyanine staining: For the analysis of CNS demyelination, eriochrome-cyanine (EC) staining was performed. The histological sections were dried for 2 hours at room temperature (RT) and another 2 hours in a stove at 37° C. The slides were immersed in cold acetone for 5 minutes at RT, and allowed to aerate for 30 minutes for the acetone to evaporate. Subsequently, the cuts were immersed in a staining solution containing 0.2% Eriochrome-Cyanine (Sigma), 0.5% sulfuric acid (H₂SO₄, Sigma) and 4% ferric aluminum (Sigma, 10% prepared in distilled water), in distilled water for 30 minutes at RT. Excess dye was removed and the cuts were immersed in an aqueous solution of 5% ferric aluminum (Sigma) in distilled water for 10 minutes at RT for differentiation of dyed tissue. The excess dye was removed again with water and they were immersed in a solution of borax-ferrocyanide for 10 minutes at RT. After washing with water, the correct differentiation of the staining was verified under light field microscope (myelinated areas stained in blue and white or yellowish demyelinated areas). For its conservation, the sections were dehydrated in ethanol solutions of increasing concentration at 70, 80, 90, 96 and 100% ethanol, rinsed in two 100% xylene baths and mounted with mounting medium.

Immunohistochemistry: For the detection of antigens present in the sections, they were allowed to defrost and dry at RT for 1 hour. Then, a pretreatment with 10% methanol in 0.1 M PB (phosphate buffer) was performed at RT for 15 minutes and under stirring. After two washes of 0.1 M PB and 1×PBS of 10 minutes each, incubation was carried out in blocking solution to avoid nonspecific binding (5% of normal donkey serum (NDS; Millipore) and 0.02% of Triton X-100 (Sigma-Aldrich) in 1×PBS, for one hour, in the dark, wet chamber and RT After this, the sections were incubated in the mixture of primary antibodies (MBP (rat, Serotec), NFH (rabbit, Abcam), Ibal (guinea pig, Synaptic System), Olig2 (rabbit, Millipore), CC1 (mouse, Millipore) and PDGFRα (goat, Rd)) in the corresponding blocking solution for 12 hours, at 4° C. and in a humid chamber, development was performed by using secondary fluorescence antibodies in the blocking solution at a concentration of 1:1000 for one hour at RT. The sections were then stained with HOECHST® (Sigma) for core development at a concentration of 1:10 relative to the stock (100 μg/ml of Bisbenzimide, Sigma-Aldrich, in milliQ water) in PBS and for 10 minutes at RT, in darkness and in wet chamber. Finally, the sections were washed in PBS and mounted with cub Slides in FLUOROMONT® mounting medium (SouthernBiotech).

Image and analysis: A Leica SP-5 confocal microscope was used to take images of the spinal cord cuts. Three photos (mosaics) were taken per animal with a separation between planes of 3 μm at a magnification of 40× and a resolution of 512×512 pixels. For the analysis of the area of NFH, Ibal and MBP as well as the demyelinated area of cyanine eriochrome staining, the Image J application was used. IMARIS Software for 3D and 4D Imaging was used to count microglia cells and nuclei.

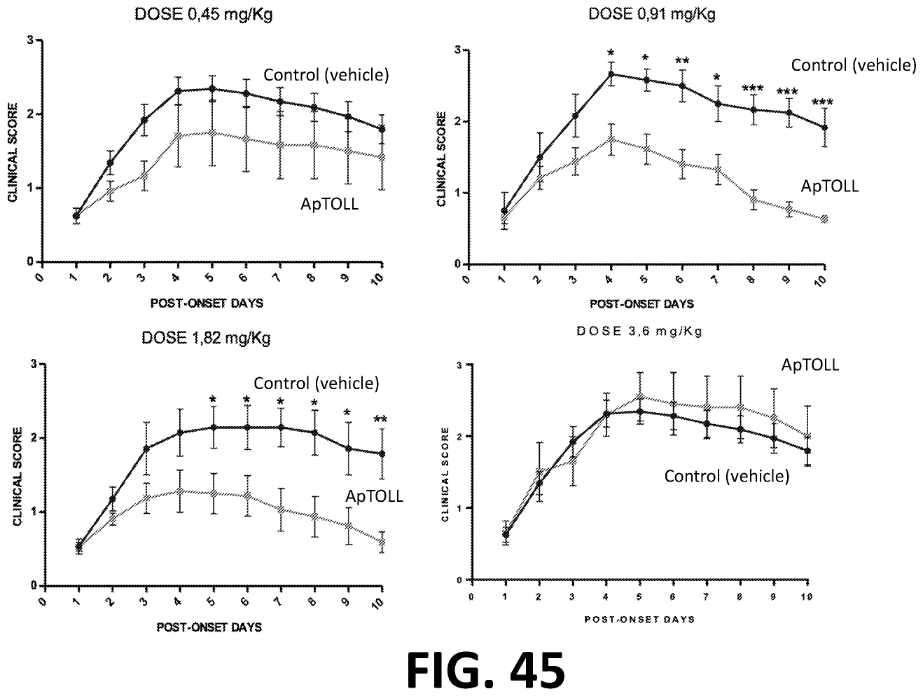

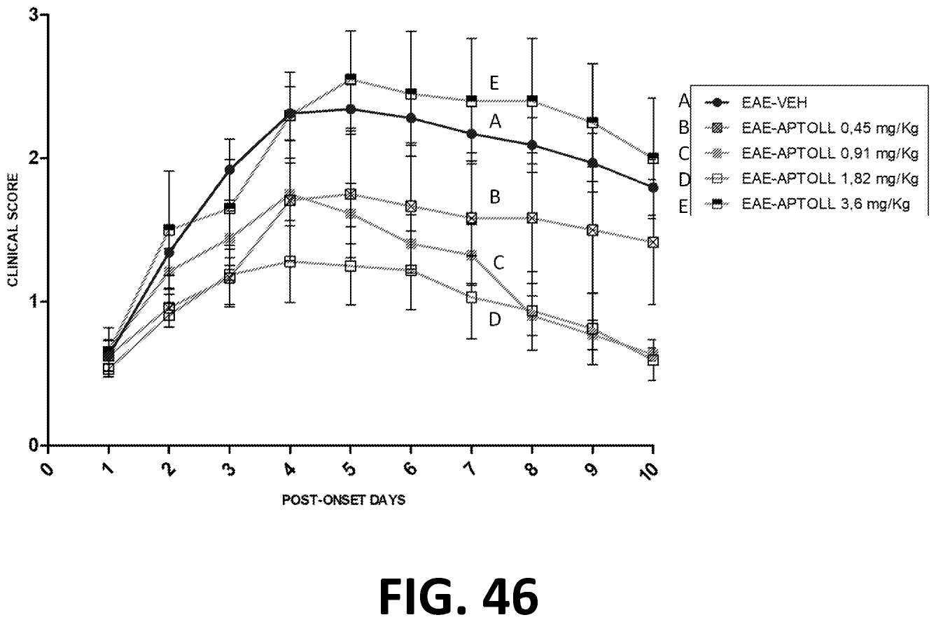

Results: As seen in FIGS. 45 and 46, with a dose of 0.45 mg/kg, there was an improvement in the clinical score in the EAE-ApTOLL group. With 0.91 mg/kg ApTOLL dose, the clinical course of EAE was significantly improved at 4 days after the injection of ApTOLL at the time of the onset of symptoms. With 1.82 mg/kg ApTOLL dose, the clinical score significantly improved 5 days after the injection of ApTOLL with respect to the vehicle group. With 3.6 mg/kg ApTOLL dose, the clinical score is slightly higher in the EAE-ApTOLL group than in the vehicle. Therefore, treatment in particular with the doses of 0.91 mg/Kg and 1.82 mg/Kg resulted in a significant reduction in the slope of the EAE clinical course curve. This difference in the score reached by the animals was significant at 4 days after the start in the case of the dose of 0.91 mg/Kg and 5 days in the case of the dose of 1.82 mg/Kg, until the end of the experiment, respect to the group of vehicles. The histological analysis of the spinal cord of mice receiving the 0.91 mg/Kg and 1.82 mg/Kg dose was performed to study the remyelating and neuroprotective effect of this compound and compare both concentrations.

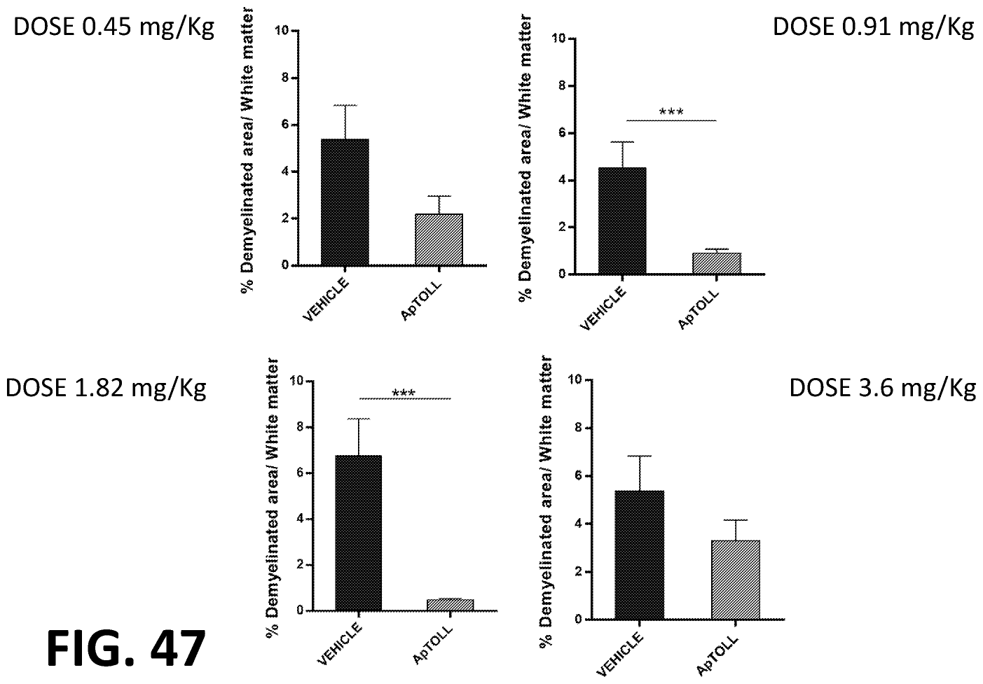

Demyelinated areas of the spinal cord sections were analyzed by eriochrome-cyanine staining (FIG. 47), observing a higher percentage of demyelinated area (not shown) of the white matter in the vehicle group than in those animals treated with ApTOLL. Thus, ApTOLL protects from demyelinating processes during EAE because shows less % demyelinated area than the vehicle.

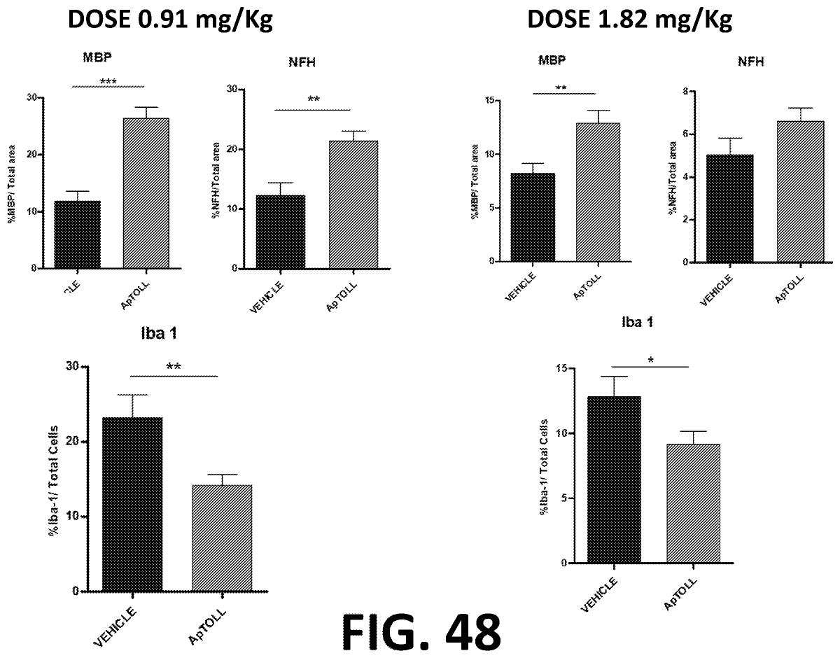

Next, different markers were analyzed in those two doses in which the differences between the treated group and the vehicle were greater (0.91 mg/kg and 1.82 mg/kg), to study the possible neuroprotective effect that ApTOLL might have in this model. The area of myelin existing in the white matter of the spinal cord was studied using the MBP marker (Myelin Basic Protein), and the neuroaxonal damage was studied using the NFH marker (Neurofilament Heavy Polypeptide Protein, marker for axonal damage) and the level of inflammation produced at the time of sacrifice was studied using a marker for the activated microglia cells (Ibal, Calcium Binding protein specific for microglia). In both doses, a higher percentage of both MBP and NFH was observed in the EAE-ApTOLL group and a significant decrease in Ibal with respect to the vehicle group (FIG. 48). This indicated that ApTOLL at these two concentrations had a neuroprotective effect in this animal model of MS with inflammatory component.

To determine the optimal concentration of ApTOLL, the ratio of (re)myelination, neuroprotection and inflammation between the two selected doses was calculated. The results showed an improved dose effect for the 0.91 mg/kg dose than for the 1.82 mg/kg dose.

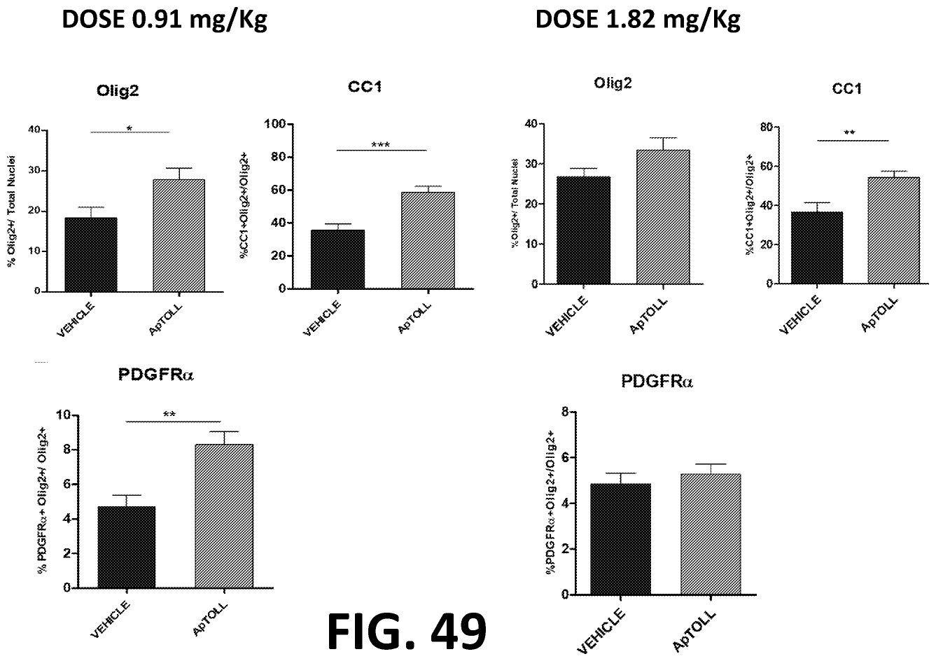

To determine if ApTOLL improved proliferation and differentiation of myelin-forming cells, a histological study of the oligodendroglial lineage was carried out. For each dose, a larger amount of precursor oligodendrocytes (PDGFRα+ cells, proliferation marker) and mature oligodendrocytes (CC1+ cells, differentiation marker) was observed in the EAE-ApTOLL group with respect to the vehicle treated group (FIG. 49), confirming the remyelinating effect observed previously by using the myelin marker MBP.

Conclusions: The dose-response study of ApTOLL by testing four different doses of ApTOLL in the animal model EAE of MS indicated that the optimal dose for such treatment could be 0.91 mg/kg, with a second option as 1.82 mg/kg. At the time of the appearance of the symptoms in the EAE model, ApTOLL administration produced a considerable weight recovery and a significant reduction in the clinical course in the treated animals compared to the animals in the vehicle group. Furthermore, ApTOLL administration had a clear effect on tissue remyelination, in addition to producing a significant reduction in axonal damage and inflammation. The administration of these two doses resulted in a reduction in demyelinated area as well as an increased in the myelinated area and amount of neurofilaments unlike the vehicle.

9.4. ApToll Used in this Study

| Name | TLR4-4FT |
|---|---|
| Required quantity | Monodoses of 0.91 mg/kg |
| Packaging | Microcentrifugue tube |
| Aspect | Colorless lyophilized powder |
| Conditions of storage | −20° C. protected from the light |
| Protocol of preparation | 1. Centrifuge the vials so that all the solid |
| | remains at the bottom and there are no losses |
| | when opening it |
| | 2. Add 1 ml of sterile nuclease-free water, cover, |
| | shake until completely dissolved (do not sonicate) |
| | 3. Centrifuge so that the entire volume remains |
| | at the bottom of the tube |
| | 4. Stabilize 5 minutes at room temperature |
| | 5. Take the entire volume and transfer to a tube |
| | with 14 ml of PBS + 1 mM Cl₂Mg and mix by |
| | inversion. |
| | 6. Take the necessary volume to inject the animal |
| Solubility | Aqueous up to 0.05 nmol/μl |
| Time of preparation | 10 minutes before use |

### Example 10. Efficacy of ApTOLL on the Promyelinating in an In Vitro Model of Oligodendrocyte Precursor Cells (OPCs) of Rats

This study was done blindly, and the potential promyelinating effect of ApTOLL on OPCs extracted from rats of 7-days-old was assayed. To this end, the survival, proliferation, and differentiation of the OPCs after the treatment with ApTOLL was measured.

Survival assay: The OPCs were plated 24 h before the treatment with control, vehicle (PBS with 10 mM MgCl₂), ApTOLL (20 nM-200 nM), and positive control for death with H₂O₂. After incubation for 24 h, the % of cell survival was measured by MTT kit, as manufacturer specifications, and the signal measured at 595 nm.

Proliferation assay: The OPCs were plated 24 h before the treatment with control, vehicle (PBS with 10 mM MgCl₂), and ApTOLL (20 nM-200 nM). Then, a BrdU pulse was given to cells for 6 h. The cells were fixed in a paraformaldehyde solution 24 h later, and the immunocytochemistry was done using BrdU and Olig2 as primary antibodies. To quantify the proliferating cells, the images were visualized by confocal microscopy.

Differentiation assay: The OPCs were plated and grown for 24 h. Then, the media was replaced by media supplemented with grown factors (PDGF-α y FGF2) and treated with control (media without grown factors), vehicle (PBS with 10 mM MgCl₂), ApTOLL (20 nM-200 nM), and positive control for differentiation with Thyroid hormone (T3). The cells were fixed in paraformaldehyde solution to perform the immunocytochemistry with the primary antibodies MBP and Olig2. To quantify the proliferating cells, the images were visualized by confocal microscopy.

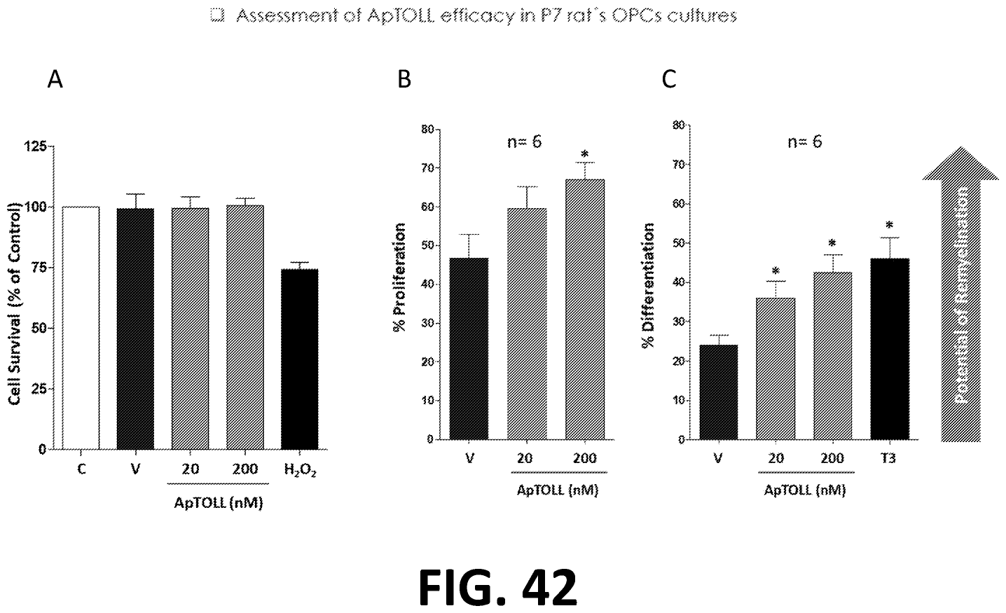

The treatment with ApTOLL showed a dose-dependent effect on OPCs proliferation and differentiation. At the 200 nM ApTOLL treatment condition, the cell proliferation, and differentiation were increased by 43.2% and 53.6% respectively. However, no impact on cell survival was observed (FIG. 42). These data indicated a role of ApTOLL in processes that lead to promyelination in a MS model. Such effect may also be beneficial for the treatment of neuronal tissue damage associated with other TLR-4 mediated conditions such as ischemic stroke, hemorrhagic transformation, hemorrhagic stroke, or myocardial infarction.

### Example 11. Efficacy of ApTOLL to Treat Hemorrhagic Stroke or Hemorrhagic Transformation

Hemorrhagic stroke and hemorrhagic transformation are related to physiological changes also observed in ischemic stroke, in the same tissue (brain tissue), and also are known to be TLR-4 mediated conditions. Accordingly, the experimental methods disclosed above for the characterization of the effect of aptamers of the present disclosure on ischemic stroke and other methods known in the art will be used to determine the effect of the aptamers of the present disclosure (e.g., ApTOLL) to treat hemorrhagic stroke and hemorrhagic transformation, and symptoms and sequelae thereof.

It is expected that the administration of the aptamers of the present disclosure (e.g., ApTOLL) to subjects having hemorrhagic stroke or hemorrhagic transformation will result in (i) reduction in damaged tissue; (ii) reduction in inflammation; (iii) improvement in prognosis and outcome; (iv) decrease in levels in proinflammatory biomarkers (e.g., interferon-gamma, interleukin-12p70, TNFalpha, IL-6, or any combination thereof); (v) increase in quality of life; (vi) improvement in functional scores, e.g., motor scores (e.g., an improvement in mobility); (vii) increase in survival; or, (v) any combination thereof.

### Example 12. Study of the ApTOLL Treatment's Effect on Cardiac Function and Tissue Damage after Myocardial Infarction Through an Experimental Procedure of Ischemia and Coronary Reperfusion in Swine

Test Item: ApTOLL was formulated as freeze-dried monodose vials, previously dissolved in PB S+1 mM Cl₂Mg and structured.

| Name | TLR4-4FT |
|---|---|
| Required quantity | Monodoses of 0.078 mg/kg |
| Packaging | Microcentrifugue tube |
| Aspect | Colorless lyophilized powder |
| Conditions of storage | −20° C. protected from the light |
| Protocol of preparation | Same as section 9.4. of this document (Example 9) |
| Solubility | Aqueous up to 0.05 nmol/μl |
| Time of preparation | 10 minutes before use |

Vehicle: A solution of PBS plus 1 mM Cl₂Mg was prepared as vehicle. The dosage (0.078 mg/kg) was calculated according to the weight of the animal.

Materials: Hematoxylin-eosin, Trichrome Masson staining reagents, TTC, Evans Blue and fetal bovine serum were from Sigma. Horse radish peroxidase (HRP)-conjugated anti-mouse secondary antibody and liquid 3,3′-diaminobenzidine (DAB) substrate were from Dako. Anti-MMP-9 antibody and Human Cardiac Troponin 1 Simple-Step ELISA Kit (ab200016) were from Abcam, proteome Profiler Array (ARY005B) was from R&D System, ketamine was from Pfizer, midazolam was from Braun, isoflurane was from Abbvie, propocol was from Fresenius, fentanyl was from Kern Pharma, diazepam was from Roche, amiodarone was from Sanofi Aventis. All the catheters were from Cordis. The following is a list of the most commonly equipment used for this study: 5415R Refrigerated Centrifuge was from Eppendorf (Hamburg, Germany). The chemiluminescence imaging system Fusion Solo-S and the image analysis software Fusion-Capt were from Vilber-Lourmat (Eberhardzell, Germany). TCS-SP5 Confocal Microscope was from Leica (Wetzlar, Germany). Microplate reader was from Biotek (Winooski, Vt.). NanoDrop One Spectrophotometer was from Thermo Scientific (Waltham, Mass.). Guiding catheters, angioplasty balloons and catheter introducers were from Cordis (Miami, Fla.). Diagnostic and steerable guidewires were from Boston Scientifics (Malborough, Mass.) and the balloon inflation devices were from Braun (Melsungen, Germany).

Protocols: Animals and Cardiogenic Shock (CS) Procedure

Yorkshire female pigs (37.8±5.2 kg) were pre-medicated with intramuscular ketamine (10 mg/kg, Pfizer) and midazolam (0.5 mg/kg, Braun). Anesthesia was induced by inhaled isoflurane (Abbvie Spain SLU) and maintained with continuous infusion of propofol (2 ml/kg/h, Fresenius Kabi), fentanyl (50 μg/kg/h, Kern Pharma) and diazepam (10 μg/kg/h, Roche). Animals were intubated and ventilated with 100% oxygen saturation. The animals received 5000 IU of heparin and amiodarone (2 mg/kg/h, Sanofi Aventis) to avoid blood clotting of catheters and malignant cardiac arrhythmias, respectively.

Ischemia/Reperfusion was induced by left anterior descending artery (LAD) occlusion for 45 minutes, using a JL 3 6F catheter and an angioplasty balloon (inflated to the pressure of 8 atmospheres). In cases when ventricular fibrillation/ventricular tachycardia occurred, we administered biphasic DC shock (10-20 joules) combined with direct manual chest compressions. After 45 minutes of LAD occlusion, the artery was unblocked and after 10 minutes of reperfusion, the treatment was administered intravenously. The treatment was blind administered, 10 animals were treated with ApTOLL and 10 with control vehicle. 10 mL of arterial Blood were obtained from the femoral artery at the following times: Before AMI, 50 minutes, 75 minutes, 2 hours, 8 hours, 24 hours, 3 days and 7 days post AMI. Immediately after collection, plasma was isolated by centrifugation at 3000 rpm for 10 minutes and all the samples were kept at −80° C. before experimentation and stored for future investigation.

Echocardiography: Pig hearts were visualized by echocardiography by using a Vivid Q ultrasound system (GE healthcare) equipped with a 1.9-4 MHz scan head. In anesthetized animals parasternal short-axis-view images of the heart were recorded in a B-mode to allow M-mode recordings by positioning the cursor in the parasternal short-axis view perpendicular to the interventricular septum and posterior wall of the left ventricle. From these recordings, the following parameters were determined using the on-site software cardiac package: systolic and diastolic Interventricular septum thickness (IVS), systolic and diastolic left-ventricle internal diameter (LVID), systolic and diastolic left-ventricle posterior Wall thickness (LVPW), left-ventricle ejection fraction (EF), left ventricle shortening fraction (FS), heart rate (HR), and cardiac output (CO).

Double Evans Blue/TTC staining: The extension of myocardial infarction was evaluated by Evans blue perfusion and TTC staining. By day 7, a catheter inflated at same position as in day 0 to avoid Evans blue perfusion downstream to the area at risk, and a pigtail catheter was inserted from the femoral artery and placed up to the left ventricle for Evans blue perfusion into the systemic circulation. One minute after perfusion, the animals were sacrificed by injection of a potassium chloride solution, and the hearts were then isolated, washed 3 times with saline buffer, frozen for 12 hours at −20° C., and chopped into 0.5 cm slices from base to apex. The slices were incubated with 1% TTC dye dissolved in saline buffer for 20 minutes at 37° C., and then washed for 20 minutes with 10% paraformaldehyde. Images were acquired by confocal microscopy and analyzed with the ImageJ software, discriminating between healthy areas from the area at risk and the pale necrotic area, calculating the area of necrosis as percentage respect to the area at risk.

Confocal microscopy: Paraffin embedded 0.5 μm heart sections were incubated with anti-MMP-9 (diluted 1:500 in PBS 1.5% BSA) primary antibody overnight at 4° C. After washing 3 times with PBS, the slides were incubated with ALEXA-FLUOR-647™ conjugated secondary antibody for 1 hour at room temperature. Slides were washed 3 times with PBS, and mounted in PBS media containing HOECHST™ for nuclei visualization. Images were taken using a Leica TCS SP5 confocal microscope. At least three different fields per condition were obtained.

Determinations. Plasma levels of Troponin I determination: Plasma Troponin I was determined with the commercial kit Human Cardiac Troponin 1 SIMPLESTEP™ ELISA Kit from Abcam following the manufacturer's instructions.

Histology and immunohistofluorescence: Heart morphology was visualized by Eosin-Hematoxylin staining, and collagen deposition was detected by Masson's trichrome staining. Immunohistochemical detection of MMP-9 was performed by incubating samples with the corresponding primary and secondary antibodies and detecting bound fluorescence conjugated secondary antibodies by confocal microscopy. Densitometric determinations of signals were evaluated by using the non-commercial software ImageJ.

Statistical analysis: All values were given as mean±S.D. Significance is reported at the 5% level. Whenever comparisons were made with a common control, significance of differences was tested by Dunnett's modification of the t test.

Results:

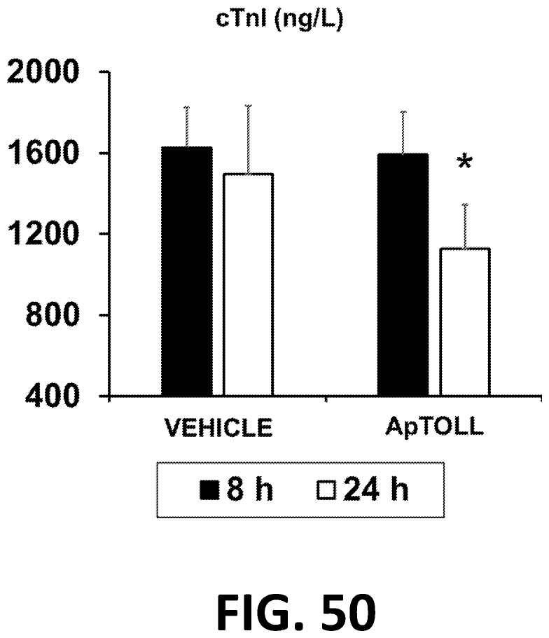

ApTOLL reduced Troponin I levels 24 h after reperfusion. Cardiac Troponin I (TpnI) is very useful in the study of allegedly angina chest pain, as it has a high sensitivity and specificity for the detection of ischemic injury, which is why it is routinely used in those patients who come with or without a previous diagnosis of acute coronary syndromel. TpnI values were equally high in both groups of animals after 8 hours of procedure, although after 24 hours, there was a significant decrease of 29.5% in the group treated with ApTOLL (FIG. 50).

ApTOLL induced recovery of heart function by day 7 after reperfusion. To assess whether cardiac function was affected 3 and 7 days after reperfusion, the following parameters were determined by echocardiography in anesthetized pigs:

IVSD: End diastolic interventricular septum thickness.
LVIDD: End diastolic internal diameter of the left ventricle.
LVPWD: End diastolic left ventricular posterior wall thickness.
IVSS: End systolic interventricular septum thickness.
LVIDS: End systolic internal diameter of the left ventricle.
LVPWS: End systolic left ventricular posterior wall thickness.
EF: Left ventricle ejection fraction.
FS: Shortening fraction.
HR: Heart rate.

### CO: Cardiac Output.

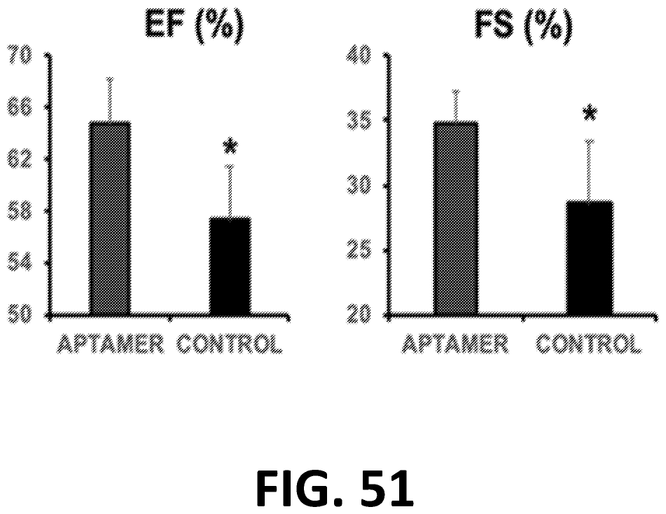

No differences were found in response to ApTOLL administration 3 days after reperfusion (data not shown). However, by day 7 EF and FS, were significantly increased in response to ApTOLL (FIG. 51).

ApTOLL reduced left ventricle necrosis and fibrosis by day 7 after reperfusion. This test assessed whether ApTOLL could effectively reduce myocardial necrosis. A surgical procedure was performed to detect the healthy perfused region of the heart (healthy area), the risk perfused region (area at risk), and the infarction non-perfused region (necrotic area). This surgical approach allowed to use the risk zone as a percentage of the infarct area, to be able to avoid differences due to the size of each heart and the specific region where the coronary occlusion was performed.

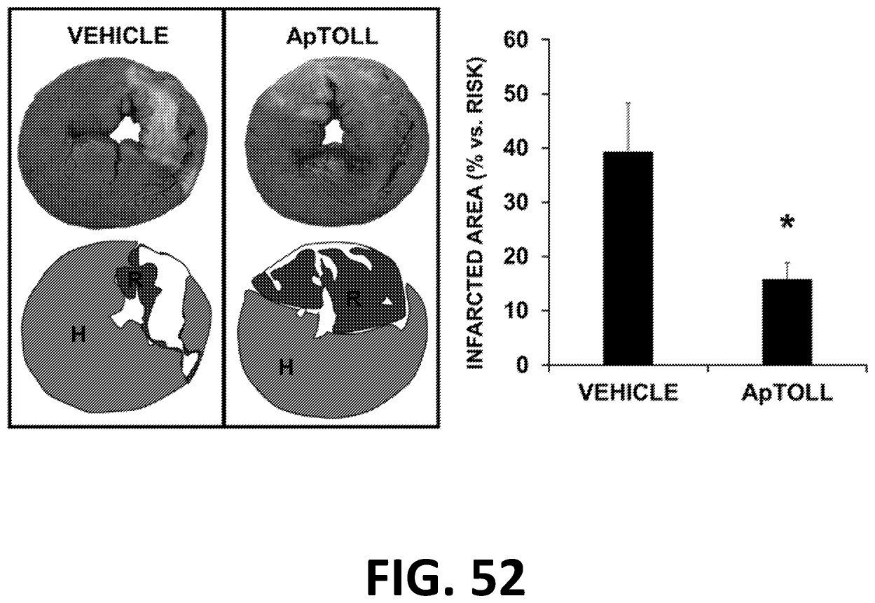

After 7 days of treatment, the animals were subjected to a double catheterization, accessing and occluding the anterior descending coronary artery at the same point as day zero through one femoral artery, and accessing the left ventricle with a “pigtail” catheter through the other femoral artery. Once the coronary artery was occluded in the same region as day 0, “Evans Blue” dye was injected through the pigtail catheter into the left ventricle, with the aim of perfusing it into the animal. In this way, specifically in the heart, the healthy area of the tissue was stained in blue, except for the area at risk when the coronary artery was occluded by the balloon. Finally, after the animal was sacrificed, the heart was isolated in diastole (by injection of potassium chloride) and transversely chopped in sections 0.5 cm thick. The sections were incubated with the TTC reagent, which was internalized into non-necrotic cells, staining them in red (risk zone), while the necrotic cells were refractory to staining (infarct zone, white). Once the procedure was carried out, it was possible to detect a significant reduction of the infarct area of approximately 28% (with respect to the area at risk) in the animals treated with ApTOLL (FIG. 52, left panel). FIG. 52, right panel demonstrates that the infarcted area observed in ApTOLL is 42% smaller than that observed when vehicle alone is used.

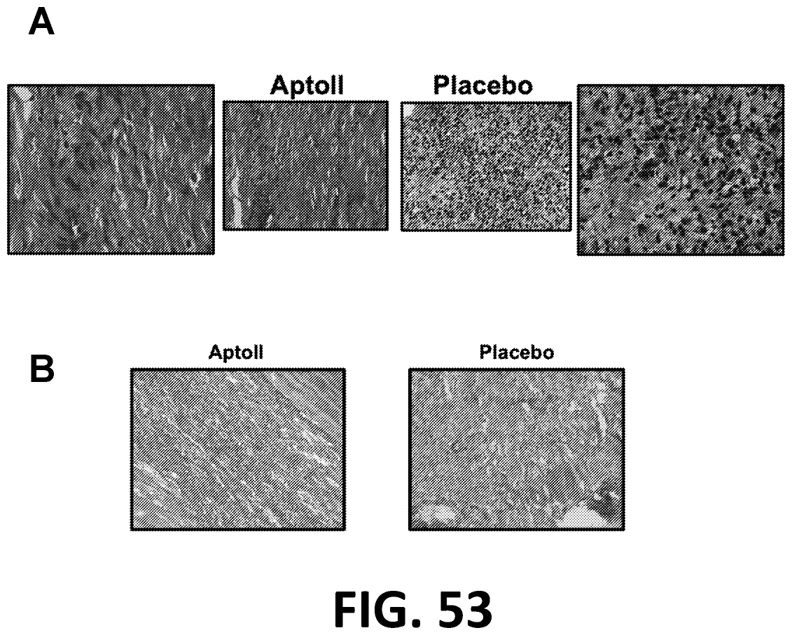

To visualize the integrity of the heart's myocardial fibers, and also to evaluate the presence of foci of inflammation in response to ischemia and subsequent coronary reperfusion, the hearts were sliced in 0.5-micron cross sections for eosin-hematoxylin staining, detecting how treatment with ApTOLL maintained the integrity of the myocardial tissue, along with a reduction in the number of infiltrates (FIG. 53, panel A). Masson's trichrome staining revealed significant myocardial fibrosis in the hearts of Placebo treated animals (FIG. 53, panel B).

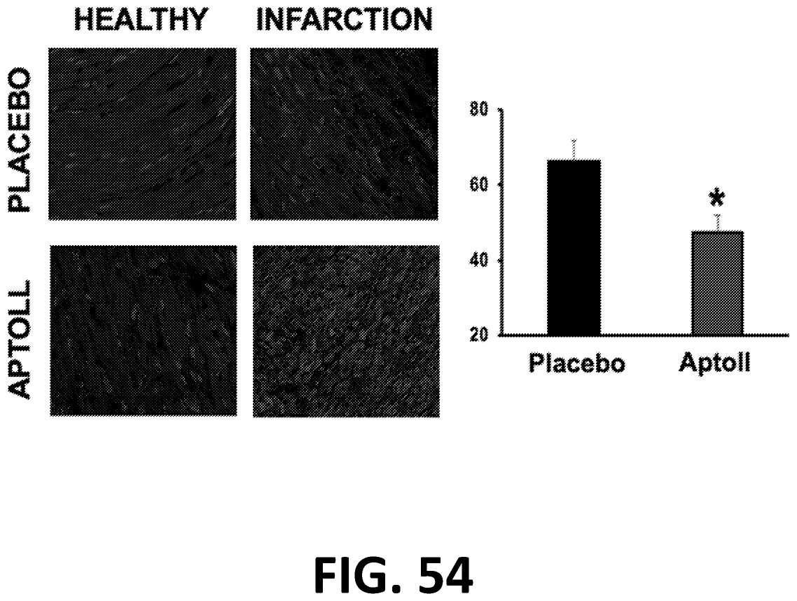

ApTOLL inhibited the expression of MMP-9 by day 7 after reperfusion. Degradation of the extracellular matrix is a critical step during myocardial remodeling and repair. Matrix Metallo-Protease 9 (MMP-9), a marker of adverse remodeling, plays a key role in this process as it degrades the extracellular matrix components. In pigs treated with ApTOLL, the expression of MMP-9 by day 7 after reperfusion was significantly reduced by a 40% with respect to the Placebo group, as detected in the same heart sections previously described, by confocal microscopy immunofluorescence using specific anti-MMP-9 antibodies (FIG. 54).

Conclusions: Altogether these results show that ApTOLL is a suitable therapeutic agent for the treatment of AMI. Others have shown that TLR4 is correlated with a bad outcome in acute myocardial infarction (AMI; heart attack). The present study shows that the treatment with ApTOLL induces cardioprotective effects by (i) downregulating the inflammatory process, (ii) reducing the degradation of extracellular matrix and therefore improving the myocardial remodeling, preserving the ventricular anatomy and cardiac function and (iii) reducing the progression of the infarction.

### Example 13. Tissue Distribution of ApTOLL by qPCR

ApTOLL labeled with the HILYTE™ FLUOR 488 dye (0.45 mg/kg) or vehicle were administered i.v. to Wistar male rats (8-10 weeks old), in order to quantify the aptamer in different tissues, namely, heart, lung, kidney, spleen, liver, small intestine, large intestine, pancreas, thymus and ependymal fat. The following groups were analyzed:
NV: naïve rats treated with vehicle (n=2).
N-1 h rats: naïve rats treated with ApTOLL (n=2). Tissues were collected 1 h after injection.
N-24 h rats: naïve rats treated with ApTOLL (n=2). Tissues were collected 24 h after injection.
I-1 h rats: ischemic rats treated with ApTOLL 10 min after occlusion (pMCAO by electrocoagulation; n=2). Tissues were collected 1 h after injection.
I-24 h rats: naïve rats treated with ApTOLL 10 min after occlusion (pMCAO by electrocoagulation; n=2). Tissues were collected 24 h after injection.

In the specific case of ApTOLL assessment in the brain, 6 ischemic (W1-6) and 1 naïve rat were injected with ApTOLL (0.45 mg/kg, i.v. 10 min after occlusion) and brain was collected 1 h later. All animals were anesthetized and euthanized at the times described by cardiac perfusion. Tissues were washed with saline infusion, collected, and immediately frozen at −80° C.

In the second part of the study, the tissue of each organ was thawed and weighted. Approximately 100 mg of each tissue was processed with 1 mL of Nucleozol (Macherey-Nagel) except thymus and heart (atrium) in which, weighing approximately 50 mg, 500 μl of nucleozole was used to obtain the RNA. For all extractions, RNA levels were measured and their integrity was checked in 1.2% agarose gels.

A volume of RNA (25 μl) was treated with RNAse A for 30 min and ApTOLL levels were determined by qPCR with the appropriate primers and using the kit “AceQ qPCR SYBR® Green Master Mix, Vazyme” in a real-time thermal cycler One Sep Plus (Applied Biosystems). Increasing concentrations of ApTOLL-HILYTE-488 (0.001-10 fmoles) were used as the standard pattern. The amount of aptamer/g of tissue was calculated.

Results and Conclusions:

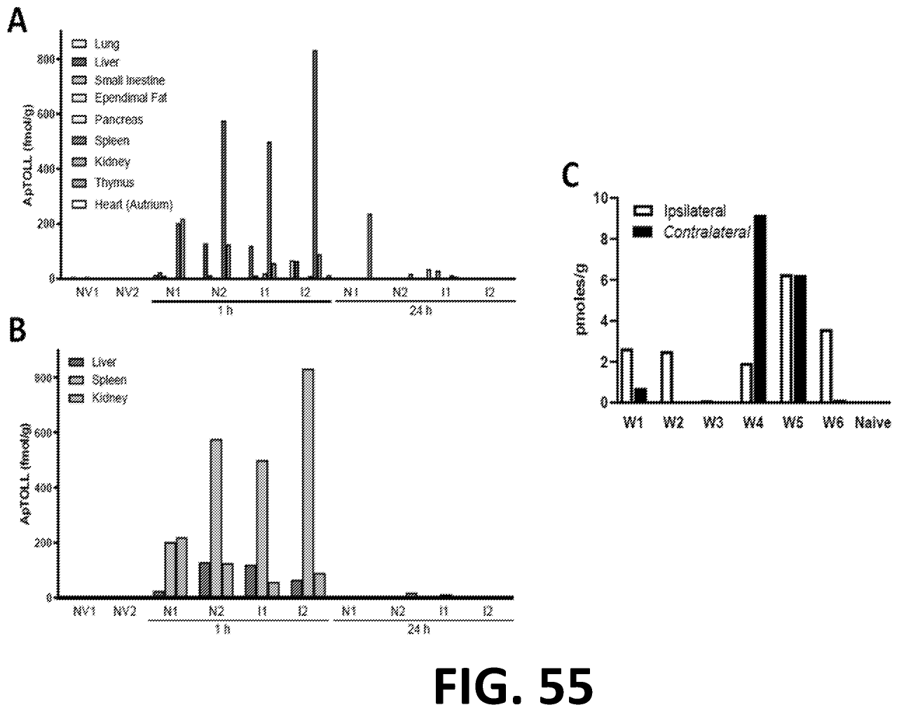

1. ApTOLL was mainly present in kidney, spleen and liver 1 h after injection, both in naïve and ischemic rats. However, 24 h after injection, ApTOLL levels were almost undetectable (FIG. 55, panels A, B).

2. In the brain, ApTOLL was detectable in ischemic (mainly in the ipsilateral hemisphere) but not in naïve rats, confirming that ApTOLL was not able to cross the BBB under physiological conditions (FIG. 55, panel C).

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

1-30. (canceled)

31. A method of improving cardiac function after cardiac infarction in a subject in need thereof, the method comprising administering an aptamer to the subject after the cardiac infarction wherein
(a) the aptamer has a length between 40 and 100 nucleotides and is selected from the group consisting of SEQ ID NOS: 1, 2, 3, and 4, wherein
(i) the aptamer specifically binds to an epitope on the extracellular domain of TLR-4; and,
(ii) binding of the aptamer to the epitope reduces and/or inhibits TLR-4 activation; or
(b) the aptamer is a functional equivalent variant of the aptamer of (a) having at least 85% sequence identity to SEQ ID NO: 1, 2, 3, or 4, wherein the functionally equivalent variant is derived from SEQ ID NO: 1, 2, 3, or 4, and maintains the capability of specifically binding to and reducing and/or inhibiting TLR-4 activation.

32. The method of claim 31, wherein the administration of the aptamer results in an improvement in cardiac function selected from the group consisting of (i) reduction of the infarcted area; (ii) decrease in fibrosis and/or necrosis; (iii) reduction in degradation of extracellular matrix; (iv) improvement in cardiac remodeling; (v) preservation of ventricular anatomy; (vi) reduction of progression of the infarction; and, (vii) any combination thereof.

33. The method of claim 31, wherein the aptamer is administered in combination with artery recanalization.

34. The method of claim 33, wherein artery recanalization is surgical, pharmacological, or a combination thereof.

35. The method of claim 34, wherein the surgical artery recanalization is catheterization.

36. The method of claim 35, wherein the catheterization is balloon catheterization, stent catheterization, or a combination thereof.

37. The method of claim 34, wherein the pharmacological artery recanalization is pharmacological thrombolysis or pharmacomechanical thrombolysis.

38. The method of claim 33, wherein the administration of the aptamer takes place prior, during, and/or after artery recanalization.

39. The method of claim 33, wherein the aptamer is administered at least 10 minutes after artery recanalization.

40. The method of claim 33, wherein the aptamer is administered at least 30 minutes prior to artery recanalization.

41. The method of claim 33, wherein the aptamer is administered prior and immediately after performing artery recanalization.

42. The method of claim 33, wherein the aptamer is administered at least 30 minutes prior to artery recanalization and about 10 minutes after artery recanalization.

43. The method of claim 31, wherein the aptamer is administered intravenously by infusion.

44. The method of claim 43, wherein the infusion has a duration of about 5 minutes, about 10 minutes, about 15 minutes, about 20 minutes, about 25 minutes, or about 30 minutes.

45. The method according to claim 32, wherein the reduction of the infarcted area is by at least 25%, at least 30%, at least 35%, at least 40%, at least 45% or at least 50% compared to control conditions.

46. The method according to claim 32, wherein the decrease in fibrosis and/or necrosis can be observed 1 day, 2 days, 3 days, 4 days, 5 days, 6 days, 7 days, 8 days, 9 days, 10 days, 11 days, 12 days, 13 days, or 14 days after administering the aptamer.

47. The method of claim 31, wherein the aptamer is ApTOLL (SEQ ID NO: 1).

48. The method of claim 31, wherein the aptamer is administered at a dose range between 0.5 mg/dose and 10 mg/dose.

49. The method of claim 31, wherein the aptamer is administered at a dose range between 0.007 mg/kg per dose and 0.14 mg/kg per dose.

50. The method of claim 31, wherein the aptamer is formulated in phosphate buffered saline (PBS), pH 7.4, comprising magnesium chloride, and optionally A-trehalose.


SEQUENCE LISTING (16 sequences)

SEQ ID NO: 1 - DNA, 45 bp, Artificial Sequence
  nucleic acid aptamer
ccggcacgggacaaggcgcgggacggcgtagatcaggtcgacacc

SEQ ID NO: 2 - DNA, 59 bp, Artificial Sequence
  nucleic acid aptamer
ggtgtgccaataaaccatatcgccgcgttagcatgtactcggttggccctaaatacgag

SEQ ID NO: 3 - DNA, 78 bp, Artificial Sequence
  nucleic acid aptamer
gttgctcgtatttagggccaccggcacgggacaaggcgcgggacggcgtagatcaggtcgacaccagtcttcatccgc

SEQ ID NO: 4 - DNA, 76 bp, Artificial Sequence
  nucleic acid aptamer
gcggatgaagactggtgtgccaataaaccatatcgccgcgttagcatgtactcggttggccctaaatacgagcaac

SEQ ID NO: 5 - DNA, 78 bp, Artificial Sequence; identical to SEQ ID NO: 3
  nucleic acid aptamer

SEQ ID NO: 6 - DNA, 78 bp, Artificial Sequence
  nucleic acid aptamer
gcggatgaagactggtgtcgacctgatctacgccgtcccgcgccttgtcccgtgccggtggccctaaatacgagcaac

SEQ ID NO: 7 - DNA, 68 bp, Artificial Sequence
  nucleic acid aptamer
gttgctcgtatttagggcacacacgcacgaagaccttggctgcccgttgtacaccagtcttcatccgc

SEQ ID NO: 8 - DNA, 68 bp, Artificial Sequence
  nucleic acid aptamer
gcggatgaagactggtgtacaacgggcagccaaggtcttcgtgcgtgtgtgccctaaatacgagcaac

SEQ ID NO: 9 - DNA, 76 bp, Artificial Sequence
  nucleic acid aptamer
gttgctcgtatttagggcaccgaggtcaccgaacttggtgtgcacagttgttggcgcgacaccagtcttcatccgc

SEQ ID NO: 10 - DNA, 76 bp, Artificial Sequence
  nucleic acid aptamer
gcggatgaagactggtgtcgcgccaacaactgtgcacaccaagttcggtgacctcggtgccctaaatacgagcaac

SEQ ID NO: 11 - DNA, 76 bp, Artificial Sequence
  nucleic acid aptamer
gttgctcgtatttagggccacatatgtgcacatcacaatccgcagagctgcacctacgacaccagtcttcatccgc

SEQ ID NO: 12 - DNA, 76 bp, Artificial Sequence
  nucleic acid aptamer
gcggatgaagactggtgtcgtaggtgcagctctgcggattgtgatgtgcacatatgtggccctaaatacgagcaac

SEQ ID NO: 13 - DNA, 76 bp, Artificial Sequence
  nucleic acid aptamer
gttgctcgtatttagggccaaggaaaaccccctggtcactggtactaatccgatccgtacaccagtcttcatccgc

SEQ ID NO: 14 - DNA, 76 bp, Artificial Sequence
  nucleic acid aptamer
gcggatgaagactggtgtacggatcggattagtaccagtgaccagggggttttccttggccctaaatacgagcaac

SEQ ID NO: 15 - DNA, 76 bp, Artificial Sequence
  nucleic acid aptamer
gttgctcgtatttagggcgggtcaccacggaagagtgtagatacatagatacagtccgacaccagtcttcatccgc

SEQ ID NO: 16 - DNA, 76 bp, Artificial Sequence
  nucleic acid aptamer
gcggatgaagactggtgtcggactgtatctatgtatctacactcttccgtggtgacccgccctaaatacgagcaac